SEQUENCE LISTING (3 sequences)

SEQ ID NO: 1 - DNA, 20 bp, Artificial Sequence
  primer
gtgtctgcggcgttttatca

SEQ ID NO: 2 - DNA, 21 bp, Artificial Sequence
  primer
gacaaacgggcaacatacctt

SEQ ID NO: 3 - DNA, 28 bp, Artificial Sequence
  probe
cctctkcatcctgctgctatgcctcatc

(19) United States
(12) Patent Application Publication
(10) Pub. No.: US 2022/0267313 A1
(43) Pub. Date: Aug. 25, 2022

(54) DIHYDROPYRIMIDINE DERIVATIVES AND USES THEREOF IN THE TREATMENT OF HBV INFECTION OR OF HBV-INDUCED DISEASES

(71) Applicant: Janssen Sciences Ireland Unlimited Company, Co Cork (IE)

(72) Inventors: Yimin JIANG, Londonderry, NH (US); Zhanling CHENG, Shanghai (CN); Gang DENG, Shanghai (CN); Zhiguo LIU, Shanghai (CN); Chao LIANG, Shanghai (CN); Jianping WU, Shanghai (CN); Linglong KONG, Shanghai (CN); Xiangjun DENG, Shanghai (CN); Yanping XU, Noblesville, IN (US)

(21) Appl. No.: 17/597,897

(22) PCT Filed: Jul. 30, 2020
(86) PCT No.: PCT/CN2020/105767

§ 371 Date: Jan. 28, 2022

(30) Foreign Application Priority Data
Jul. 31, 2019 (CN) PCT/CN2019/098575

(51) Int. Cl. C07D 417/04; C07D 417/14; A61K 31/506; A61K 45/06; C07D 403/04; C07D 401/04; A61P 31/20

(52) U.S. Cl. CPC C07D 417/04; C07D 417/14; A61K 31/506; A61K 45/06; C07D 403/04; C07D 401/04; A61P 31/20

(57) ABSTRACT

Provided herein are dihydropyrimidine derivatives which are useful in the treatment or prevention of HBV infection or of HBV-induced diseases, more particularly of HBV chronic infection or of diseases induced by HBV chronic infection, as well as pharmaceutical or medical applications thereof.

### BACKGROUND

Chronic hepatitis B virus (HBV) infection is a significant global health problem, affecting over 5% of the world population (over 350 million people worldwide and 1.25 million individuals in the U.S.).

Despite the availability of a prophylactic HBV vaccine, the burden of chronic HBV infection continues to be a significant worldwide medical problem, due to suboptimal treatment options and sustained rates of new infections in most parts of the developing world.

Current treatments do not provide a cure and are limited to only two classes of agents (interferon alpha and nucleoside analogues/inhibitors of the viral polymerase); drug resistance, low efficacy, and tolerability issues limit their impact. The low cure rates of HBV are attributed at least in part to the fact that complete suppression of virus production is difficult to achieve with a single antiviral agent. However, persistent suppression of HBV DNA slows liver disease progression and helps to prevent hepatocellular carcinoma. Current therapy goals for HBV-infected patients are directed to reducing serum HBV DNA to low or undetectable levels, and to ultimately reducing or preventing the development of cirrhosis and hepatocellular carcinoma.

The HBV capsid protein plays essential functions during the viral life cycle. HBV capsid/core proteins form metastable viral particles or protein shells that protect the viral genome during intercellular passage, and also play a central role in viral replication processes, including genome encapsidation, genome replication, and virion morphogenesis and egress.

Capsid structures also respond to environmental cues to allow un-coating after viral entry. Consistently, the appropriate timing of capsid assembly and dis-assembly, the appropriate capsid stability and the function of core protein have been found to be critical for viral infectivity.

Background references on dihydropyrimidine derivatives in the treatment of HBV infection include WO 2014/029193, CN103664899, CN103664925, and CN103664897.

There is a need in the art for therapeutic agents that can increase the suppression of virus production and that can treat, ameliorate, or prevent HBV infection. Administration of such therapeutic agents to an HBV infected patient, either as monotherapy or in combination with other HBV treatments or ancillary treatments, will lead to significantly reduced virus burden, improved prognosis, diminished progression of the disease and enhanced seroconversion rates.

### SUMMARY

Provided, in one aspect, is a compound of Formula (I)

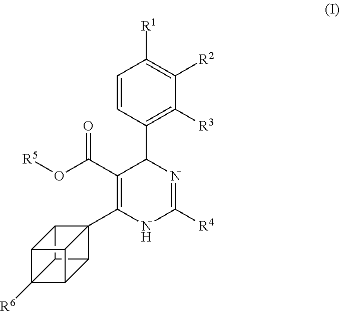

including the deuterated, stereoisomeric or tautomeric forms thereof, wherein:

R¹, R² and R³ are each independently selected from the group consisting of hydrogen, C₁₋₃ alkyl and halogen;

R⁴ is selected from the group consisting of thiazolyl, imidazolyl, oxazolyl and pyridyl, each of which may be optionally substituted with one or more substituents each independently selected from methyl or halo;

R⁵ is C₁₋₄alkyl;

R⁶ is selected from the group consisting of —CO₂R^{x}, —C₁₋₉alkyl-CO₂R^{x}, and -Het-CO₂R^{x};

wherein

C₁₋₉alkyl may be optionally substituted with one or more substituents, each independently selected from halo and hydroxyl;

R^{x} is selected from H and —C₁₋₆alkyl; in particular, H and —C₁₋₄alkyl; and

Het represents a 5- to 6-membered aromatic ring in which 1, 2, 3 or 4 of the ring members is a heteroatom each independently selected from the group consisting of N, O, and S, wherein said 5- to 6-membered aromatic ring is optionally substituted with one or more substituents, each independently selected from C₁₋₄alkyl and halo;

or a pharmaceutically acceptable salt or a solvate thereof.

In another aspect, provided herein is a pharmaceutical composition comprising at least one compound of Formula (I), or a pharmaceutically acceptable salt thereof, together with a pharmaceutically acceptable carrier.

In another aspect, provided herein is a pharmaceutical composition comprising at least one disclosed compound, together with a pharmaceutically acceptable carrier. In another aspect, provided herein is a method of treating an HBV infection in an individual in need thereof, comprising administering to the individual a therapeutically effective amount of a compound of Formula (I) or a pharmaceutically acceptable salt thereof. In another aspect, provided herein is any of the compounds described herein, or the pharmaceutical composition of the invention, for use as a medicament. In a further aspect, provided herein is any of the compounds described herein, or the pharmaceutical composition of the invention, for use in the prevention or treatment of an HBV infection or of an HBV-induced disease in mammal in need thereof.

In yet a further aspect, provided herein is a product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound of Formula (I) or the pharmaceutical composition according to the invention, as described herein, and wherein said second compound is an HBV inhibitor. Said HBV inhibitor may be chosen from among:
cytokines having HBV replication inhibition activity,
antibodies having immune checkpoint modulation activity,
substituted pyrimidines having HBV capsid assembly inhibition activity or having TLR agonist activity,
antiretroviral nucleoside analogues, and
the combinations thereof.

In another aspect, provided herein is a method of inhibiting or reducing the formation or presence of HBV DNA-containing particles or HBV RNA-containing particles in an individual in need thereof, comprising administering to the individual a therapeutically effective amount of a compound of Formula (I), or a pharmaceutically acceptable salt thereof.

In an embodiment, any of the methods provided herein can further comprise administering to the individual at least one additional therapeutic agent selected from the group consisting of an HBV polymerase inhibitor, immunomodulatory agents, interferon, viral entry inhibitor, viral maturation inhibitor, capsid assembly modulator, reverse transcriptase inhibitor, a cyclophilin/TNF inhibitor, a TLR-agonist, an HBV vaccine, and any combination thereof.

In a still further aspect, a process is provided for producing the compound of Formula (I), the process comprising:
(i) reacting a compound of Formula (IV)

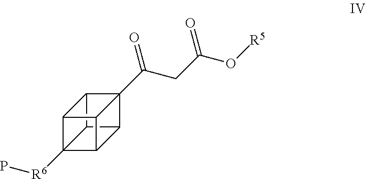

with a compound of formula (V)

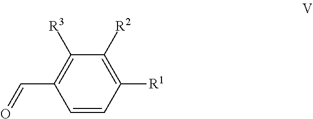

under suitable condensation conditions to yield a compound of Formula (X)

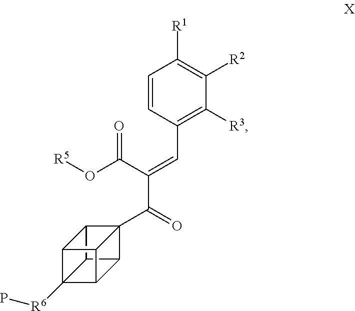

reacting the compound of Formula (X) with a compound of Formula (VI)

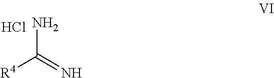

under basic conditions to generate a compound of Formula (VII)

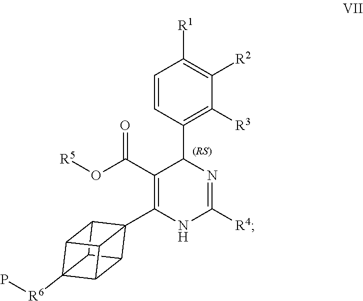

or alternatively
(ii) reacting the compounds of Formula (IV), (V) and (VI) in the presence of a suitable base in a suitable solvent to yield the compound of formula (VII); and optionally comprising the step of
(iii) resolving the compound of Formula (VII) into compounds of Formula (VIIa) and (VIIb)

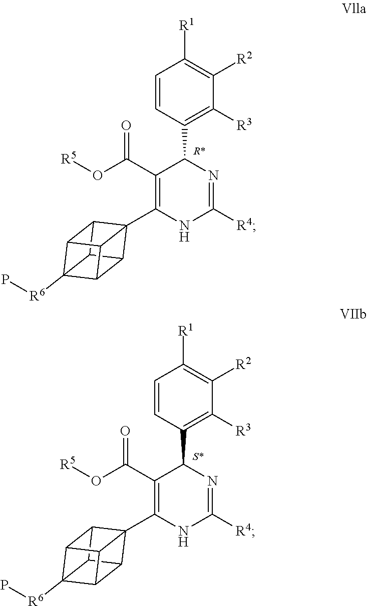

by chiral separation; and the step of
(iv) cleaving the protecting group in compound (VII) to yield compound of Formula (I); or
cleaving the protecting group in compound (VIIa) or compound (VIIb) to yield a compound of Formula (Ia) or (Ib)

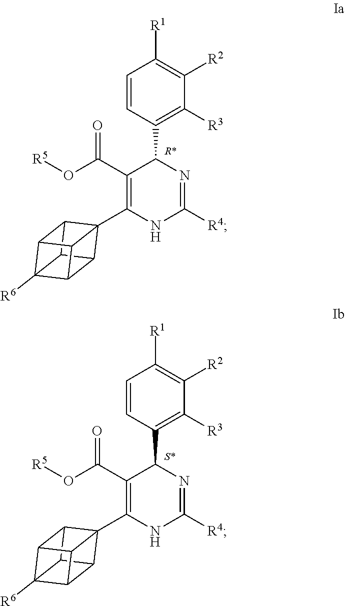

wherein R¹, R², R³, R⁴, R⁵, and R⁶ are as defined herein for the compound of Formula (I), and P is a suitable protecting group.

### DESCRIPTION

Provided herein are compounds, e.g., the compounds of Formula (I), or pharmaceutically acceptable salts thereof, that may be useful in the treatment and prevention of HBV infection in a subject.

Without being bound to any particular mechanism of action, these compounds are believed to modulate or disrupt HBV assembly and other HBV core protein functions necessary for HBV replication or the generation of infectious particles and/or may disrupt HBV capsid assembly leading to empty capsids with greatly reduced infectivity or replication capacity. In other words, the compounds provided herein may act as capsid assembly modulators.

There is still a need for compounds with HBV antiviral activity with an advantageous balance of properties, for example potent antiviral activity, favorable metabolic properties, tissue distribution, safety and pharmaceutical profiles, and are suitable for use in humans. It is accordingly an object of the present invention to provide compounds that overcome at least some of these problems. The disclosed compounds may modulate (e.g., accelerate, delay, inhibit, disrupt or reduce) normal viral capsid assembly or disassembly, bind capsid or alter metabolism of cellular polyproteins and precursors. The modulation may occur when the capsid protein is mature, or during viral infectivity. Disclosed compounds can be used in methods of modulating the activity or properties of HBV cccDNA, or the generation or release of HBV RNA particles from within an infected cell.

In one embodiment, the compounds described herein may be suitable for monotherapy and may be effective against natural or native HBV strains and against HBV strains resistant to currently known drugs. In another embodiment, the compounds described herein may be suitable for use in combination therapy.

### Definitions

Listed below are definitions of various terms used to describe this invention. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used herein, the term “capsid assembly modulator” refers to a compound that disrupts or accelerates or inhibits or hinders or delays or reduces or modifies normal capsid assembly (e.g., during maturation) or normal capsid disassembly (e.g., during infectivity) or perturbs capsid stability, thereby inducing aberrant capsid morphology and function. In one embodiment, a capsid assembly modulator accelerates capsid assembly or disassembly, thereby inducing aberrant capsid morphology. In another embodiment, a capsid assembly modulator interacts (e.g. binds at an active site, binds at an allosteric site, modifies or hinders folding and the like) with the major capsid assembly protein (CA), thereby disrupting capsid assembly or disassembly. In yet another embodiment, a capsid assembly modulator causes a perturbation in structure or function of CA (e.g., ability of CA to assemble, disassemble, bind to a substrate, fold into a suitable conformation, or the like), which attenuates viral infectivity or is lethal to the virus.

As used herein, the term “treatment” or “treating” is defined as the application or administration of a therapeutic agent, i.e., a disclosed compound (alone or in combination with another pharmaceutical agent), to a patient, or application or administration of a therapeutic agent to an isolated tissue or cell line from a patient (e.g., for diagnosis or ex vivo applications), who has an HBV infection, a symptom of HBV infection or the potential to develop an HBV infection, with the purpose to cure, heal, alleviate, relieve, alter, remedy, ameliorate, improve or affect the HBV infection, the symptoms of HBV infection, or the potential to develop an HBV infection. Such treatments may be specifically tailored or modified, based on knowledge obtained from the field of pharmacogenomics.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual” or “subject” refers to a human or a non-human mammal. Non-human mammals include, for example, livestock and pets, such as ovine, bovine, porcine, canine, feline and murine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p. 1445 and Journal of Pharmaceutical Science, 66, 1-19 (1977), each of which is incorporated herein by reference in its entirety.

As used herein, the term “composition” or “pharmaceutical composition” refers to a mixture of at least one compound useful within the invention with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1990, Easton, Pa.), which is incorporated herein by reference.

As used herein, the term “alkyl,” by itself or as part of another substituent means, unless otherwise stated, a straight or branched chain hydrocarbon having the number of carbon atoms designated (i.e., C₁₋₃alkyl means an alkyl having one to three carbon atoms, C₁₋₄alkyl means an alkyl having one to four carbons and includes straight and branched chains, C₁₋₆alkyl means an alkyl having one to six carbon atoms and includes straight and branched chains, C₁-C₉alkyl means an alkyl having one to nine carbon atoms and includes straight and branched chains). Examples include methyl, ethyl, propyl, isopropyl, butyl, isobutyl, tert-butyl. Embodiments of alkyl include, but are not limited to, C₁₋₉alkyl, C₁₋₆alkyl, C₁₋₄alkyl.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

The notation “5- to 6-membered aromatic ring in which 1, 2, 3 or 4 of the ring members is a heteroatom each independently selected from N, O, or S” refers to a heterocycle having aromatic character. Particular examples include thiazolyl, oxazolyl, pyrazolyl, thiadiazolyl, oxadiazolyl, pyridyl, and pyrimidinyl.

The notation “cubane” as used herein or as part of another group, defines pentacyclo[4.2.0.02,5.03,8.04,7]octane as a radical, such as cubane-1,4-diyl, in particular, pentacyclo[4.2.0.02,5.03,8.04,7]octane-1,4-diyl:

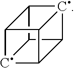

In the preparation of compounds of the present invention, protection of functional groups (e.g., carboxy) of intermediates may be necessary. The need for such protection varies depending on the nature of the functional group and the conditions of the preparation methods. The notation “protecting group” or “P” refers to a substituent that is employed to block or protect a particular functionality while reacting other functional groups on the molecule. Suitable ‘carboxy’ protecting groups include methyl, ethyl, propyl, tert-butyl (therefore, with the carboxylic acid, for example, protected as a C₁₋₄-carboxylic ester). The need for such protection is readily determined by one skilled in the art. For a general description of protecting groups and their use, see T. W. Greene and P. G. M. Wuts, Protective Groups in Organic Synthesis, 4th ed., Wiley, 25 Hoboken, N.J., 2007.

Whenever the term “substituted” is used in the present invention, it is meant, unless otherwise is indicated or is clear from the context, to indicate that one or more hydrogens, in particular from 1 to 3 hydrogens, preferably 1 or 2 hydrogens, more preferably 1 hydrogen, on the atom or radical indicated in the expression using “substituted” are replaced with a selection from the indicated group, provided that the normal valency is not exceeded, and that the substitution results in a chemically stable compound, i.e. a compound that is sufficiently robust to survive isolation to a useful degree of purity from a reaction mixture, and formulation into a therapeutic agent.

When two or more substituents are present on a moiety they may, unless otherwise is indicated or is clear from the context, replace hydrogens on the same atom or they may replace hydrogen atoms on different atoms in the moiety.

As used herein, the terminology “selected from . . . ” (e.g., “R¹ is selected from A, B and C”) is understood to be equivalent to the terminology “selected from the group consisting of . . . ” (e.g., “R¹ is selected from the group consisting of A, B and C”).

In an embodiment, the invention relates to a compound of Formula (I), as defined hereinbefore, wherein:

R¹, R² and R³ are each independently selected from the group consisting of hydrogen, methyl, fluoro, bromo, and chloro;

R⁴ is selected from the group consisting of thiazolyl, oxazolyl, imidazolyl and pyridyl, each of which may be optionally substituted with one or more substituents each independently selected from methyl or halo;

R⁵ is methyl, ethyl, propyl, or iso-propyl; and

R⁶ is selected from the group consisting of —CO₂R^{x}, —C₁₋₆alkyl-CO₂R^{x} in particular —C₁₋₄alkyl-CO₂R^{x}, and -Het-CO₂R^{x}; wherein C₁₋₆alkyl, in particular C₁₋₄alkyl, may be optionally substituted with one or more substituents, each independently selected from halo and hydroxyl;

R^{x} is selected from H and —C₁₋₆alkyl; in particular, H and —C₁₋₄alkyl; and

Het is a 5- to 6-membered aromatic ring selected from the group consisting of oxazolyl, thiazolyl, imidazolyl, pyridyl and pyrimidinyl, each of which may be optionally substituted with one or more substituents, each independently selected from C₁₋₄alkyl and halo.

In a particular embodiment, R¹, R² and R³ are each independently selected from the group consisting of H, halo, and methyl; and the rest of variables are as defined herein.

In a further embodiment, R¹ is hydrogen or halo, in particular hydrogen or fluoro; R² is hydrogen or halo, in particular hydrogen or fluoro; and R³ is selected from halo and methyl, in particular chloro and methyl; and the rest of variables are as defined herein.

In a particular embodiment, R⁴ is selected from the group consisting of thiazolyl, oxazolyl, imidazolyl and pyridyl, each of which may be optionally substituted with one methyl substituent; and the rest of variables are as defined herein. In particular, R⁴ is selected from the group consisting of thiazolyl, oxazolyl optionally substituted with methyl, and imidazolyl optionally substituted with methyl; and the rest of variables are as defined herein.

In a further embodiment, R⁵ is methyl or ethyl; and the rest of variables are as defined herein.

In an embodiment, R⁶ is selected from the group consisting of —CO₂R^{x}, —C₁₋₉alkyl-CO₂R^{x}, and -Het-CO₂R^{x}; wherein R^{x} is selected from H and —C₁₋₆alkyl; in particular, H and —C₁₋₄alkyl; and the rest of variables are as defined herein. In a further embodiment, R⁶ is selected from the group consisting of —CO₂H, —C₁₋₉alkyl-CO₂H, and -Het-CO₂H; and the rest of variables are as defined herein. In a further embodiment, R⁶ is selected from the group consisting of —CO₂H, —CH₂—CO₂H, and -Het-CO₂H; wherein Het is a 5-membered aromatic ring selected from the group consisting of oxazolyl, thiazolyl, and imidazolyl, each of which may be optionally substituted with one or more substituents, each independently selected from C₁₋₄alkyl and halo; and the rest of variables are as defined herein. Het is, in particular, oxazolyl.

In an embodiment, R^{x} is selected from H and —C₁₋₆alkyl; in particular, H and —C₁₋₄alkyl; more in particular, H, methyl, ethyl, propyl, tert-butyl; and the rest of variables are as defined herein.

All combinations of the foregoing embodiments are expressly included.

Preferred compounds according to the invention are compound or a stereoisomer or tautomeric form thereof with a formula as represented in the synthesis of compounds section and Table 1, and of which the activity is displayed in Table 3.

The disclosed compounds may possess one or more stereocenters, and each stereocenter may exist independently in either the R or S configuration. The stereochemical configuration may be assigned at indicated centers as (*) when the absolute stereochemistry is undetermined at the stereocenter although the compound itself has been isolated as a single stereoisomer and is enatiomerically/diastereomerically pure.

In one embodiment, compounds described herein are present in optically active or racemic forms. It is to be understood that the compounds described herein encompass racemic, optically-active, regioisomeric and stereoisomeric forms, or combinations thereof that possess the therapeutically useful properties described herein.

Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In one embodiment, a mixture of one or more isomer is utilized as the disclosed compound described herein. In another embodiment, compounds described herein contain one or more chiral centers. These compounds are prepared by any means, including stereoselective synthesis, enantioselective synthesis or separation of a mixture of enantiomers or diastereomers. Resolution of compounds and isomers thereof is achieved by any means including, by way of non-limiting example, chemical processes, enzymatic processes, fractional crystallization, distillation, and chromatography.

When the absolute R or S stereochemistry of a compound cannot be determined, it can be identified by the retention time after chromatography under particular chromatographic conditions as determined by chromatography column, eluent, etc.

In one embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In one embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements).

In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and techniques known to a person skilled in the art. General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein. General synthesis schemes are given in the Examples below.

Accordingly, a process is provided for producing the compound of Formula (I), wherein said process comprises
(i) reacting a compound of Formula (IV)

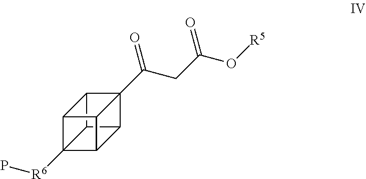

with a compound of formula (V)

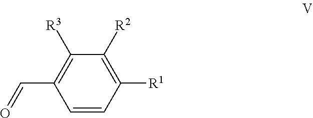

under suitable condensation conditions to yield a compound of Formula (X)

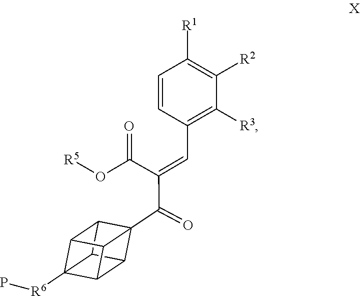

and
reacting the compound of Formula (X) with a compound of Formula (VI)

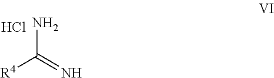

under basic conditions to generate a compound of Formula (VII)

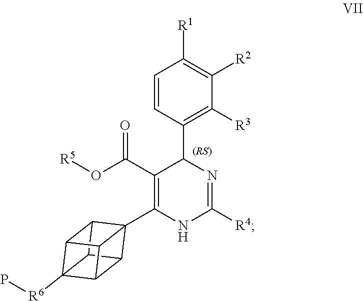

or alternatively
(ii) reacting the compounds of Formula (IV), (V) and (VI) in the presence of a suitable base in a suitable solvent to yield the compound of formula (VII); and optionally comprising the step of
(iii) resolving the compound of Formula (VII) into compounds of Formula (VIIa) and (VIIb)

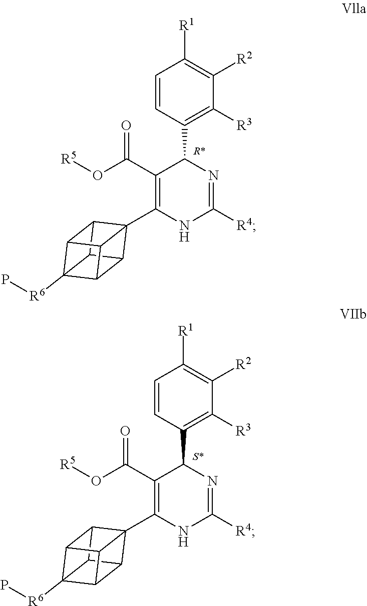

by chiral separation; and the step of
(iv) cleaving the protecting group in compound (VII) to yield compound of Formula (I); or
cleaving the protecting group in compound (VIIa) or compound (VIIb) to yield a compound of Formula (Ia) or (Ib)

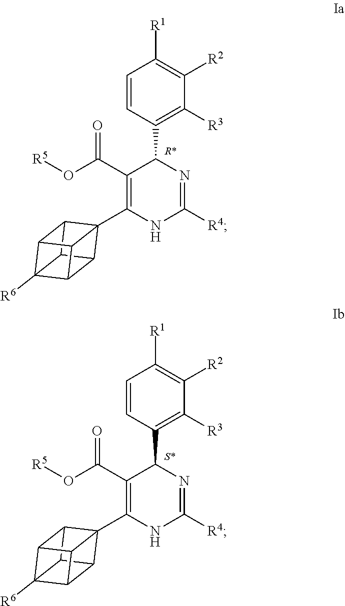

wherein R¹, R², R³, R⁴, R⁵, and R⁶ are as defined herein for the compound of Formula (I), and P is a suitable protecting group.

Methods and Uses

Provided herein is a method of treating an HBV infection in an individual in need thereof, comprising administering to the individual a therapeutically effective amount of a disclosed compound.

Also provided herein is a method of eradicating an HBV infection in an individual in need thereof, comprising administering to the individual a therapeutically effective amount of a disclosed compound.

Provided herein is a method of reducing viral load associated with an HBV infection in an individual in need thereof, comprising administering to the individual a therapeutically effective amount of a disclosed compound.

Further, provided herein is a method of reducing reoccurrence of an HBV infection in an individual in need thereof, comprising administering to the individual a therapeutically effective amount of a disclosed compound.

Provided herein is a method of inhibiting or reducing the formation or presence of HBV DNA-containing particles or HBV RNA-containing particles in an individual in need thereof, comprising administering to the individual a therapeutically effective amount of a disclosed compound.

Where the invention is said to relate to a method of treating an individual, it is understood that such method is to be interpreted in certain jurisdictions as a medical use, e.g. a compound or a composition according to the invention for use in treating an individual; or a use of the compound or the composition according to the invention, for the manufacture of a medicament, in particular for treating an individual. Therefore, for example, the invention also relates to a compound or a pharmaceutical composition as disclosed herein for use in the prevention or treatment of an HBV infection. Also provided herein, is a compound or a pharmaceutical composition as disclosed herein for use in the reduction of viral load associated with an HBV infection. Further provided herein, is a compound or a pharmaceutical composition as disclosed herein for use in the reduction of reoccurrence of an HBV infection in an individual. Also provided herein, is a compound or a pharmaceutical composition as disclosed herein, for use in the inhibition or reduction of the formation or presence of HBV DNA-containing particles or HBV RNA-containing particles in an individual.

In certain aspects, the methods, uses and/or compositions described herein are effective for inhibiting or reducing the formation or presence of HBV-associated particles in vitro or in vivo (e.g., in a cell, in a tissue, in an organ (e.g., in the liver), in an organism or the like). HBV-associated particles may contain HBV DNA (i.e., linear and/or covalently closed circular DNA (cccDNA)) and/or HBV RNA (i.e., pre-genomic RNA and/or sub-genomic RNA). Accordingly, HBV-associated particles include HBV DNA-containing particles or HBV RNA-containing particles.

As used herein, “HBV-asociated particles” refer to both infectious HBV virions (i.e., Dane particles) and non-infectious HBV subviral particles (i.e., HBV filaments and/or HBV spheres). HBV virions comprise an outer envelope including surface proteins, a nucleocapsid comprising core proteins, at least one polymerase protein, and an HBV genome. HBV filaments and HBV spheres comprise HBV surface proteins, but lack core proteins, polymerase and an HBV genome. HBV filaments and HBV spheres are also known collectively as surface antigen (HBsAg) particles. HBV spheres comprise middle and small HBV surface proteins. HBV filaments also include middle, small and large HBV surface proteins.

HBV subviral particles can include the nonparticulate or secretory HBeAg, which serves as a marker for active replication of HBV.

Provided herein is a method of reducing an adverse physiological impact of an HBV infection in an individual in need thereof, comprising administering to the individual a therapeutically effective amount of a disclosed compound.

Also provided herein is a method of reducing, slowing, or inhibiting an HBV infection in an individual in need thereof, comprising administering to the individual a therapeutically effective amount of a disclosed compound.

Provided herein is a method of inducing reversal of hepatic injury from an HBV infection in an individual in need thereof, comprising administering to the individual a therapeutically effective amount of a disclosed compound.

Provided herein is a method of reducing the physiological impact of long-term antiviral therapy for HBV infection in an individual in need thereof, comprising administering to the individual a therapeutically effective amount of a disclosed compound.

Provided herein is a method of prophylactically treating an HBV infection in an individual in need thereof, wherein the individual is afflicted with a latent HBV infection, comprising administering to the individual a therapeutically effective amount of a disclosed compound.

Also provided herein, is a compound or a pharmaceutical composition as disclosed herein, for use in the reduction of an adverse physiological impact of an HBV infection in an individual.

Also provided herein is a compound or a pharmaceutical composition as disclosed herein, for use in the reduction, slowing or inhibition of an HBV infection in an individual. Also provided herein, is a compound or a pharmaceutical composition as disclosed herein for use in inducing reversal of hepatic injury from an HBV infection in an individual.

Also provided herein is a compound or a pharmaceutical composition as disclosed herein for use in reducing the physiological impact of long-term antiviral therapy for HBV infection in an individual. Further provided herein is a compound or a pharmaceutical composition as disclosed herein for use in the prophylactic treatment of an HBV infection in an individual, wherein the individual is afflicted with a latent HBV infection.

In one embodiment, the individual is refractory to other therapeutic classes of HBV drugs (e.g, HBV polymerase inhibitors, interferons, viral entry inhibitors, viral maturation inhibitors, literature-described capsid assembly modulators, antiviral compounds of distinct or unknown mechanism, and the like, or combinations thereof). In another embodiment, the disclosed method or use reduces viral load in an individual suffering from an HBV infection to a greater extent or at a faster rate compared to the extent that other therapeutic classes of HBV drugs reduce viral load in the individual.

In one embodiment, the administering of a disclosed compound, or a pharmaceutically acceptable salt thereof, allows for administering of the at least one additional therapeutic agent at a lower dose or frequency as compared to the administering of the at least one additional therapeutic agent alone that is required to achieve similar results in prophylactically treating an HBV infection in an individual in need thereof.

In one embodiment, the administering of a disclosed compound, or a pharmaceutically acceptable salt thereof, reduces the viral load in the individual to a greater extent or at a faster rate compared to the administering of a compound selected from the group consisting of an HBV polymerase inhibitor, interferon, viral entry inhibitor, viral maturation inhibitor, distinct capsid assembly modulator, antiviral compounds of distinct or unknown mechanism, and any combination thereof.

In one embodiment, the disclosed method or use reduces viral load in an individual suffering from an HBV infection, thus allowing lower doses or varying regimens of combination therapies to be used.

In one embodiment, the disclosed method or use causes a lower incidence of viral mutation or viral resistance compared to other classes of HBV drugs, thereby allowing for long term therapy and minimizing the need for changes in treatment regimens.

In one embodiment, the administering of a compound the invention, or a pharmaceutically acceptable salt thereof, causes a lower incidence of viral mutation or viral resistance than the administering of a compound selected from the group consisting of an HBV polymerase inhibitor, interferon, viral entry inhibitor, viral maturation inhibitor, distinct capsid assembly modulator, antiviral compounds of distinct or unknown mechanism, and combination thereof.

In one embodiment, the disclosed method or use increases the seroconversion rate from HBV infected to non-HBV infected or from detectable HBV viral load to non-detectable HBV viral load beyond that of current treatment regimens. As used herein, “seroconversion” refers to the period of time during which HBV antibodies develop and become detectable.

In one embodiment, the disclosed method or use increases or normalizes or restores normal health, elicits full recovery of normal health, restores life expectancy, or resolves the viral infection in the individual in need thereof.

In one embodiment, the disclosed method or use eliminates or decreases the number of HBV RNA particles that are released from HBV infected cells thus enhancing, prolonging, or increasing the therapeutic benefit of the disclosed compounds.

In one embodiment, the disclosed method or use eradicates HBV from an individual infected with HBV, thereby obviating the need for long term or life-long treatment, or shortening the duration of treatment, or allowing for reduction in dosing of other antiviral agents.

In another embodiment, the disclosed method or use further comprises monitoring or detecting the HBV viral load of the subject, and wherein the method is carried out for a period of time including until such time that the HBV virus is undetectable.

Accordingly, in one embodiment, provided herein is a method of treating an HBV infection in an individual in need thereof, comprising administering to the individual a therapeutically effective amount of a compound of Formula (I), or a pharmaceutically acceptable salt thereof.

Accordingly, in one embodiment, provided herein is a method of treating an HBV infection in an individual in need thereof, comprising administering to the individual a therapeutically effective amount of a compound of Formula (I), or a pharmaceutically acceptable salt thereof.

In another embodiment, provided herein is a method of treating an HBV infection in an individual in need thereof, comprising administering to the individual a therapeutically effective amount of a compound of Table 1, or a pharmaceutically acceptable salt thereof.

In an embodiment of any of the methods provided herein, the method or use can further comprise monitoring the HBV viral load of the subject, wherein the method is carried out for a period of time such that the HBV virus is undetectable.

Combination Therapies

The disclosed compounds may be useful in combination with one or more additional compounds useful for treating HBV infection. These additional compounds may comprise other disclosed compounds and/or compounds known to treat, prevent, or reduce the symptoms or effects of HBV infection. Such compounds include, but are not limited to, HBV polymerase inhibitors, interferons, viral entry inhibitors, viral maturation inhibitors, literature-described capsid assembly modulators, reverse transcriptase inhibitors, immunomodulatory agents, TLR-agonists, and other agents with distinct or unknown mechanisms that affect the HBV life cycle or affect the consequences of HBV infection, e.g. the additional compounds may comprise HBV combination drugs, HBV vaccines, HBV DNA polymerase inhibitors, immunomodulators, toll-like receptor (TLR) modulators, interferon alpha receptor ligands, hyaluronidase inhibitors, hepatitis b surface antigen (HBsAg) inhibitors, cytotoxic T-lymphocyte-associated protein 4 (ipi4) inhibitors, cyclophilin inhibitors, HBV viral entry inhibitors, antisense oligonucleotide targeting viral mRNA, short interfering RNAs (siRNA) and ddRNAi endonuclease modulators, ribonucleotide reductase inhibitors, HBV E antigen inhibitors, covalently closed circular DNA (cccDNA) inhibitors, famesoid X receptor agonists, HBV antibodies, CCR2 chemokine antagonists, thymosin agonists, cytokines, nucleoprotein modulators, retinoic acid-inducible gene 1 simulators, NOD2 stimulators, phosphatidylinositol 3-kinase (PI3K) inhibitors, indoleamine-2, 3-dioxygenase (IDO) pathway inhibitors, PD-1 inhibitors, PD-L1 inhibitors, recombinant thymosin alpha-1, bruton's tyrosine kinase (BTK) inhibitors, KDM inhibitors, HBV replication inhibitors, arginase inhibitors, and other HBV drugs.

In non-limiting examples, the disclosed compounds may be used in combination with one or more drugs (or a salt thereof) selected from the group comprising:
HBV reverse transcriptase inhibitors, and DNA and RNA polymerase inhibitors.

In one embodiment, the additional therapeutic agent is an interferon. The term “interferon” or “IFN” refers to any member of the family of highly homologous species-specific proteins that inhibit viral replication and cellular proliferation and modulate immune response. Human interferons are grouped into three classes: Type I, Type II, and Type III. Recombinant forms of interferons that have been developed and are commercially available are encompassed by the term “interferon” as used herein. Subtypes of interferons, such as chemically modified or mutated interferons, are also encompassed by the term “interferon” as used herein.

Accordingly, in one embodiment, the compounds of Formula (I) can be administered in combination with an interferon.

In another embodiment, the additional therapeutic agent is selected from immune modulator or immune stimulator therapies, which includes biological agents belonging to the interferon class.

Further, the additional therapeutic agent may be an agent of distinct or unknown mechanism including agents that disrupt the function of other essential viral protein(s) or host proteins required for HBV replication or persistence.

In another embodiment, the additional therapeutic agent is an antiviral agent that blocks viral entry or maturation or targets the HBV polymerase such as nucleoside or nucleotide or non-nucleos(t)ide polymerase inhibitors.

In an embodiment, the additional therapeutic agent is an immunomodulatory agent that induces a natural, limited immune response leading to induction of immune responses against unrelated viruses. In other words, the immunomodulatory agent can effect maturation of antigen presenting cells, proliferation of T-cells and cytokine release (e.g., IL-12, IL-18, IFN-alpha, -beta, and -gamma and TNF-alpha among others).

In a further embodiment, the additional therapeutic agent is a TLR modulator or a TLR agonist, such as a TLR-7 agonist or TLR-9 agonist.

In any of the methods provided herein, the method may further comprise administering to the individual at least one HBV vaccine, a nucleoside HBV inhibitor, an interferon or any combination thereof.

In one embodiment, the methods described herein further comprise administering at least one additional therapeutic agent selected from the group consisting of nucleotide/nucleoside analogs, entry inhibitors, fusion inhibitors, and any combination of these or other antiviral mechanisms.

In another aspect, provided herein is method of treating an HBV infection in an individual in need thereof, comprising reducing the HBV viral load by administering to the individual a therapeutically effective amount of a disclosed compound alone or in combination with a reverse transcriptase inhibitor; and further administering to the individual a therapeutically effective amount of HBV vaccine.

In another aspect, provided herein is a method of treating an HBV infection in an individual in need thereof, comprising reducing the HBV viral load by administering to the individual a therapeutically effective amount of a disclosed compound alone or in combination with a antisense oligonucleotide or RNA interference agent that targets HBV nucleic acids; and further administering to the individual a therapeutically effective amount of HBV vaccine. The antisense oligonucleotide or RNA interference agent possesses sufficient complementarity to the target HBV nucleic acids to inhibit replication of the viral genome, transcription of viral RNAs, or translation of viral proteins.

In another embodiment, the disclosed compound and the at least one additional therapeutic agent are co-formulated. In yet another embodiment, the disclosed compound and the at least one additional therapeutic agent are co-administered.

For any combination therapy described herein, synergistic effect may be calculated, for example, using suitable methods such as the Sigmoid-Eₘₐₓ equation (Holford & Scheiner, 19981, Clin. Pharmacokinet. 6: 429-453), the equation of Loewe additivity (Loewe & Muischnek, 1926, Arch. Exp. Pathol Pharmacol. 114: 313-326) and the median-effect equation (Chou & Talalay, 1984, Adv. Enzyme Regul. 22: 27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

In an embodiment of any of the methods of administering combination therapies provided herein, the method can further comprise monitoring or detecting the HBV viral load of the subject, wherein the method is carried out for a period of time including until such time that the HBV virus is undetectable.

Administration/Dosage/Formulations

In another aspect, provided herein is a pharmaceutical composition comprising at least one disclosed compound, or a pharmaceutically acceptable salt thereof, together with a pharmaceutically acceptable carrier.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the invention are dictated by and directly dependent on (a) the unique characteristics of the disclosed compound and the particular therapeutic effect to be achieved, and (b) the limitations inherent in the art of compounding/formulating such a disclosed compound for the treatment of HBV infection in a patient.

In one embodiment, the compositions of the invention are formulated using one or more pharmaceutically acceptable excipients or carriers. In one embodiment, the pharmaceutical compositions of the invention comprise a therapeutically effective amount of a disclosed compound and a pharmaceutically acceptable carrier. Thus, illustrating the invention is a process for preparing a pharmaceutical composition, comprising mixing at least one pharmaceutically acceptable carrier with a therapeutically effective amount of a disclosed compound.

In some embodiments, the dose of a disclosed compound is from about 1 mg to about 2,500 mg. Similarly, in some embodiments, a dose of a second compound (i.e., another drug for HBV treatment) as described herein is less than about 1,000 mg.

In one embodiment, the present invention is directed to a packaged pharmaceutical composition comprising a container holding a therapeutically effective amount of a disclosed compound, alone or in combination with a second pharmaceutical agent; and instructions for using the compound to treat, prevent, or reduce one or more symptoms of HBV infection in a patient.

Routes of administration of any of the compositions of the invention include oral, nasal, rectal, intravaginal, parenteral, buccal, sublingual or topical. The compounds for use in the invention may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

An embodiment relates to a compound selected from the group consisting of compound satisfying the following formulae:

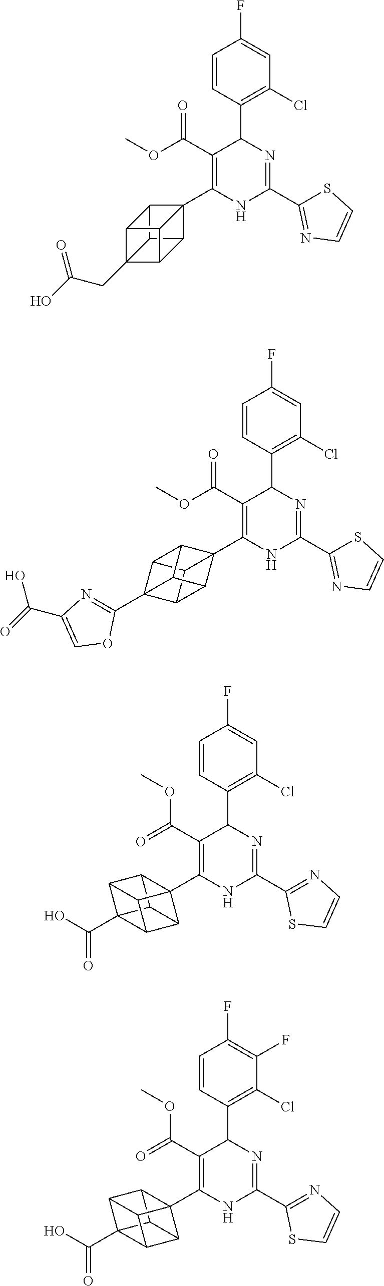 [SMILES: COC(=O)C1=C(C23C4C5C2C2C3C4C52CC(=O)O)NC(c2nccs2)=NC1c1ccc(F)cc1Cl ; COC(=O)C1=C(C23C4C5C2C2C3C4C52c2nc(C(=O)O)co2)NC(c2nccs2)=NC1c1ccc(F)cc1Cl ; COC(=O)C1=C(C23C4C5C2C2C3C4C52C(=O)O)NC(c2nccs2)=NC1c1ccc(F)cc1Cl ; COC(=O)C1=C(C23C4C5C2C2C3C4C52C(=O)O)NC(c2nccs2)=NC1c1ccc(F)c(F)c1Cl]

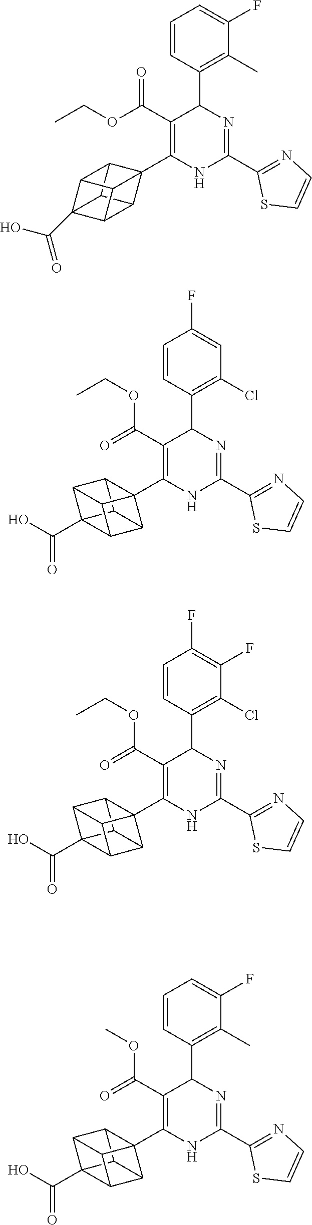 [SMILES: CCOC(=O)C1=C(C23C4C5C2C2C3C4C52C(=O)O)NC(c2nccs2)=NC1c1cccc(F)c1C ; CCOC(=O)C1=C(C23C4C5C2C2C3C4C52C(=O)O)NC(c2nccs2)=NC1c1ccc(F)cc1Cl ; CCOC(=O)C1=C(C23C4C5C2C2C3C4C52C(=O)O)NC(c2nccs2)=NC1c1ccc(F)c(F)c1Cl ; COC(=O)C1=C(C23C4C5C2C2C3C4C52C(=O)O)NC(c2nccs2)=NC1c1cccc(F)c1C]

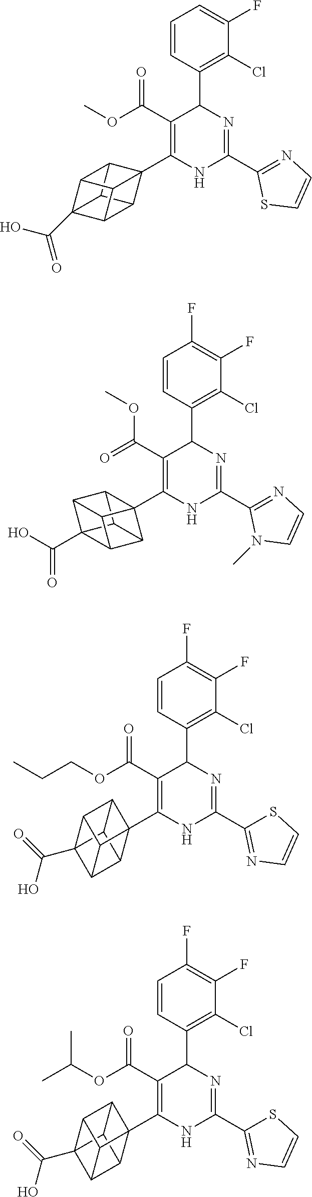 [SMILES: COC(=O)C1=C(C23C4C5C2C2C3C4C52C(=O)O)NC(c2nccs2)=NC1c1cccc(F)c1Cl ; COC(=O)C1=C(C23C4C5C2C2C3C4C52C(=O)O)NC(c2nccn2C)=NC1c1ccc(F)c(F)c1Cl ; CCCOC(=O)C1=C(C23C4C5C2C2C3C4C52C(=O)O)NC(c2nccs2)=NC1c1ccc(F)c(F)c1Cl ; CC(C)OC(=O)C1=C(C23C4C5C2C2C3C4C52C(=O)O)NC(c2nccs2)=NC1c1ccc(F)c(F)c1Cl]

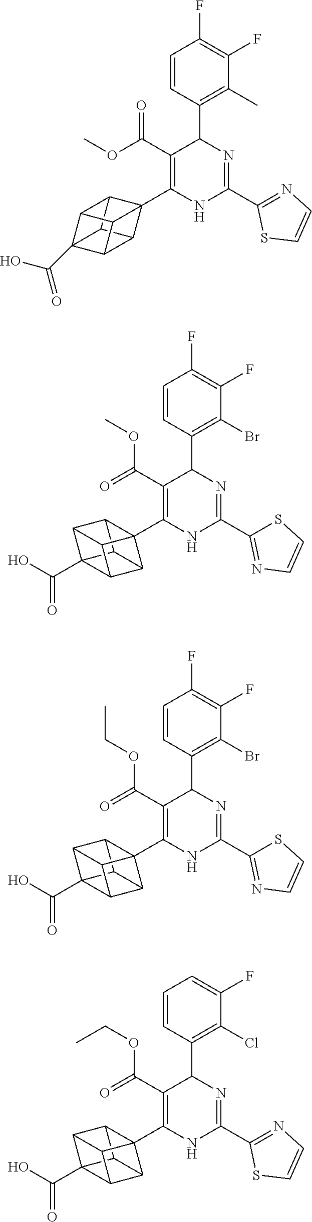 [SMILES: COC(=O)C1=C(C23C4C5C2C2C3C4C52C(=O)O)NC(c2nccs2)=NC1c1ccc(F)c(F)c1C ; COC(=O)C1=C(C23C4C5C2C2C3C4C52C(=O)O)NC(c2nccs2)=NC1c1ccc(F)c(F)c1Br ; CCOC(=O)C1=C(C23C4C5C2C2C3C4C52C(=O)O)NC(c2nccs2)=NC1c1ccc(F)c(F)c1Br ; CCOC(=O)C1=C(C23C4C5C2C2C3C4C52C(=O)O)NC(c2nccs2)=NC1c1cccc(F)c1Cl]

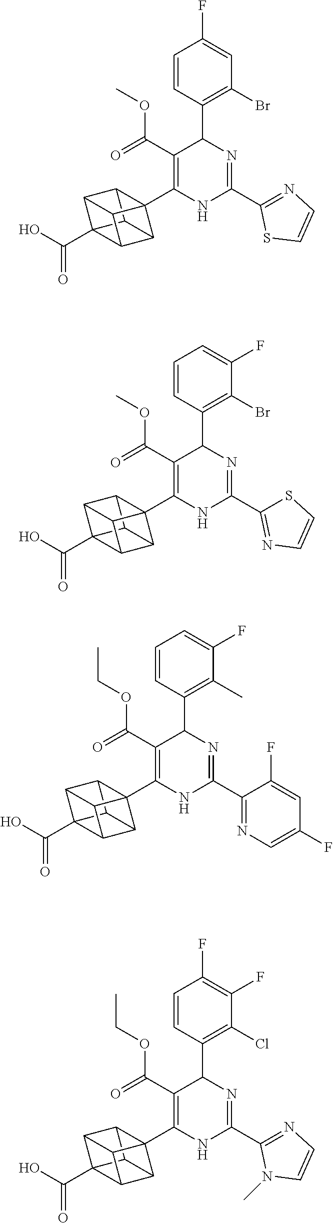 [SMILES: COC(=O)C1=C(C23C4C5C2C2C3C4C52C(=O)O)NC(c2nccs2)=NC1c1ccc(F)cc1Br ; COC(=O)C1=C(C23C4C5C2C2C3C4C52C(=O)O)NC(c2nccs2)=NC1c1cccc(F)c1Br ; CCOC(=O)C1=C(C23C4C5C2C2C3C4C52C(=O)O)NC(c2ncc(F)cc2F)=NC1c1cccc(F)c1C ; CCOC(=O)C1=C(C23C4C5C2C2C3C4C52C(=O)O)NC(c2nccn2C)=NC1c1ccc(F)c(F)c1Cl]

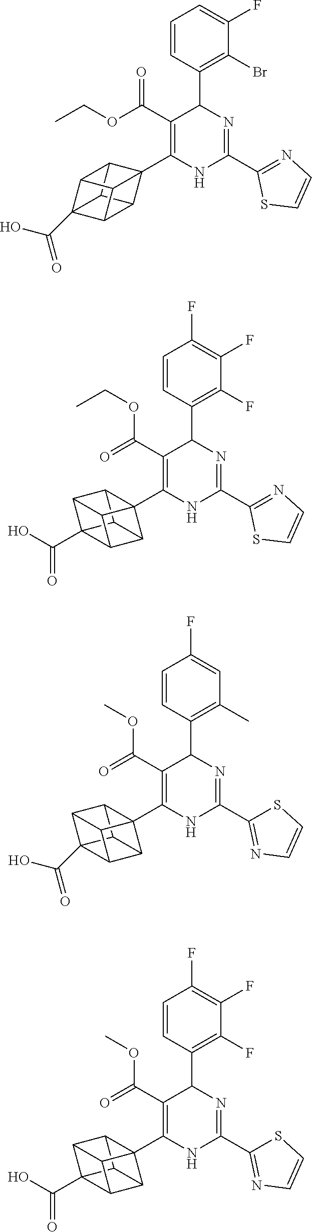 [SMILES: CCOC(=O)C1=C(C23C4C5C2C2C3C4C52C(=O)O)NC(c2nccs2)=NC1c1cccc(F)c1Br ; CCOC(=O)C1=C(C23C4C5C2C2C3C4C52C(=O)O)NC(c2nccs2)=NC1c1ccc(F)c(F)c1F ; COC(=O)C1=C(C23C4C5C2C2C3C4C52C(=O)O)NC(c2nccs2)=NC1c1ccc(F)cc1C ; COC(=O)C1=C(C23C4C5C2C2C3C4C52C(=O)O)NC(c2nccs2)=NC1c1ccc(F)c(F)c1F]

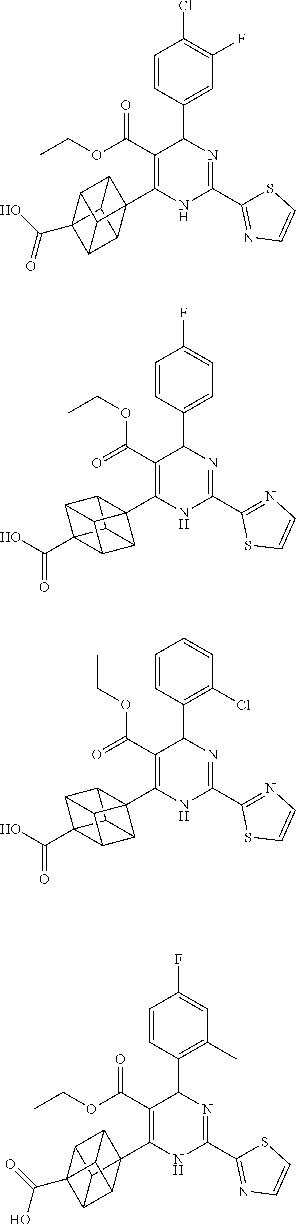 [SMILES: CCOC(=O)C1=C(C23C4C5C2C2C3C4C52C(=O)O)NC(c2nccs2)=NC1c1ccc(Cl)c(F)c1 ; CCOC(=O)C1=C(C23C4C5C2C2C3C4C52C(=O)O)NC(c2nccs2)=NC1c1ccc(F)cc1 ; CCOC(=O)C1=C(C23C4C5C2C2C3C4C52C(=O)O)NC(c2nccs2)=NC1c1ccccc1Cl ; CCOC(=O)C1=C(C23C4C5C2C2C3C4C52C(=O)O)NC(c2nccs2)=NC1c1ccc(F)cc1C]

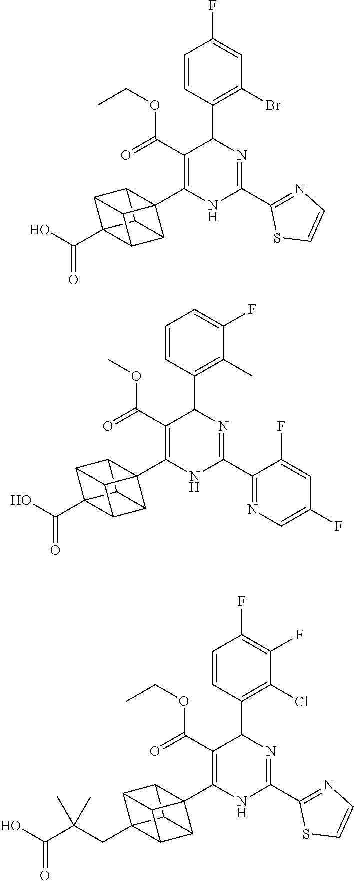 [SMILES: CCOC(=O)C1=C(C23C4C5C2C2C3C4C52C(=O)O)NC(c2nccs2)=NC1c1ccc(F)cc1Br ; COC(=O)C1=C(C23C4C5C2C2C3C4C52C(=O)O)NC(c2ncc(F)cc2F)=NC1c1cccc(F)c1C ; CCOC(=O)C1=C(C23C4C5C2C2C3C4C52CC(C)(C)C(=O)O)NC(c2nccs2)=NC1c1ccc(F)c(F)c1Cl]

### EXAMPLES

### Example 1

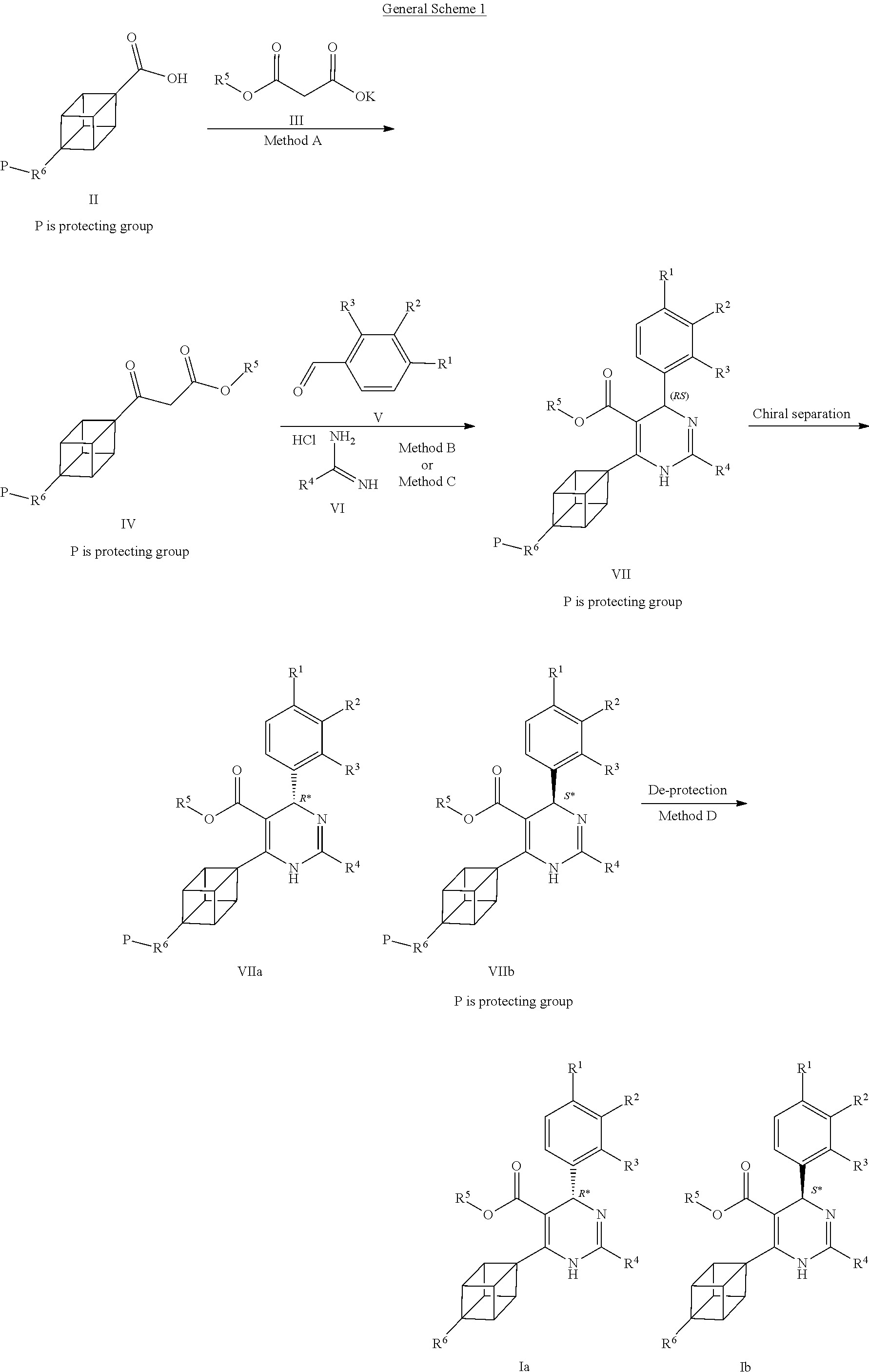

The general synthesis of compound of general formula I (Ia and Ib) is described in scheme 1. Compound of general formula IV can be synthesized as described in Scheme 1 (Method A). An acid of general formula II is converted by reacting with N,N-carbonyldiimidazole (CDI) to an activated ester which then couples with malonic acid monomethyl (or monoethyl) ester potassium salt of general formula III under basic conditions to generate an intermediate which in turn undergoes decarboxylation to yield the ketoester of general formula IV.

Compounds of general formula VII can be synthesized as described in Scheme 1 (Method B or Method C). The former is the commonly utilized chemical methodology of a stepwise approach which is provided as described in Method B. Compounds of general formula IV and V undergo condensation to yield the conjugated intermediate of formula X, which then reacts with the compound of general formula VI under a basic reaction medium at high temperature to generate the product dihydropyrimidine of general formula VII. Alternatively, compounds of general formula VII can be synthesized as described in Method C by utilizing chemical methodology of multiple component reaction with compounds of general formula IV, V and VI in the presence of base (including, but not limited to sodium acetate NaOAc) in solvent of choice (including, but not limited to ethanol EtOH). When applicable, the stereoisomers of the dihydropyrimidine product of general formula VII were isolated and purified using chiral chromatography to give the dihydropyrimidine products of general formula VIIa and general formula VIIb.

The final product of general formula I (Ia and Ib) can be synthesized through de-protection of ester hydrolysis reaction from the compounds of general formula VII (VIIa and VIIb) as described in Scheme 1 (Method D).

Method A:

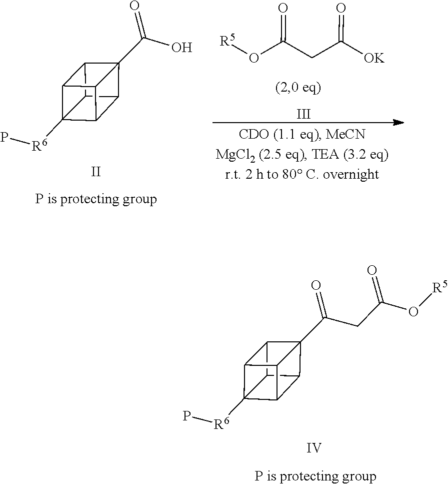

To a solution of the acid of general formula II (1 equivalent) in acetonitrile was added N,N-carbonyldiimidazole (1.1 equivalents) at room temperature. The mixture was stirred at room temperature under nitrogen atmosphere for 2 hours (mixture A). To a suspension of malonic acid monomethyl (or monoethyl) ester potassium salt (2 equivalents) of general formula III in acetonitrile was added magnesium chloride (2.5 equivalents) and triethylamine (3.2 equivalents) at room temperature. After stirred under nitrogen atmosphere for 2 hours, it was added mixture A and stirred at 80-100° C. overnight. The resulting reaction mixture was cooled down to room temperature and concentrated to give a residue, which was purified by silica gel column chromatography to afford the ketoester of general formula IV.

Method B:

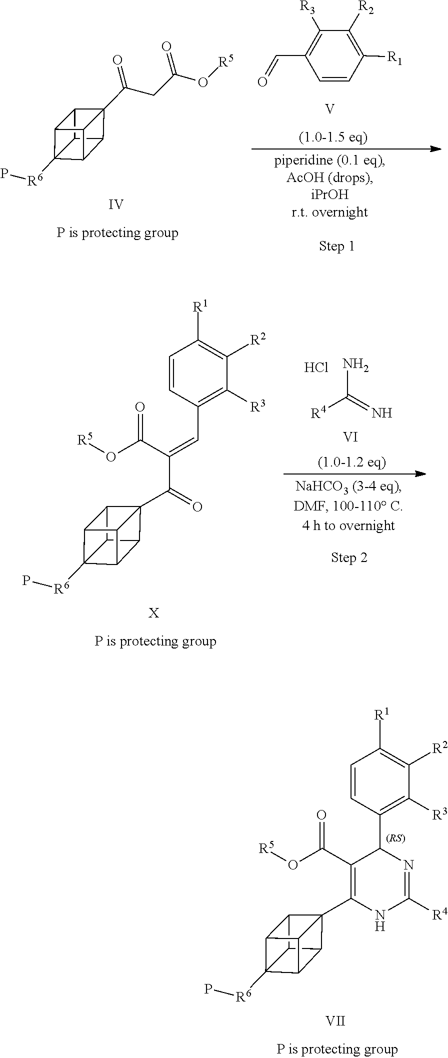

Step 1: To a solution of the ketoester of general formula IV (1 equivalent) in isopropanol was added the aldehyde of general formula V (1-1.5 equivalents), piperidine (0.1 equivalent) and acetic acid glacial (drops to 1 equivalent) at room temperature under nitrogen atmosphere. After stirred overnight, the mixture was concentrated under reduced pressure to yield a residue, which was purified by silica gel column chromatography to afford the intermediate of general formula X.

Step 2: To a solution of the intermediate of general formula X in N,N-dimethylformamide was added the carboxamidine hydrochloride of general formula VI (1-1.2 equivalents) and sodium bicarbonate (3-4 equivalents). After stirred at 100-110° C. for reaction time ranging from 4 hours to overnight, the mixture was cooled down to room temperature and concentrated under reduced pressure to leave a residue, which was purified by silica gel column chromatography to yield the dihydropyrimidine product of general formula VII. When applicable, the stereoisomers of the dihydropyrimidine product of general formula VII were isolated and purified using chiral chromatography to give the product of general formula VIIa and general formula of VIIb.

Method C:

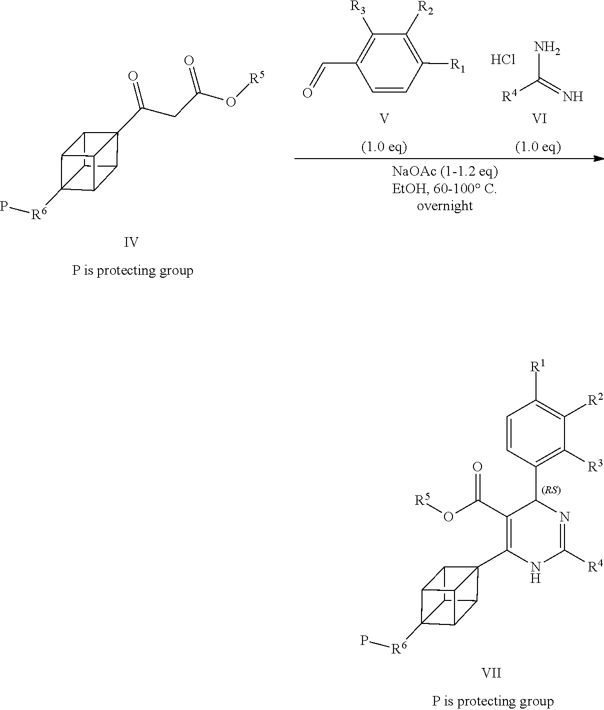

To a solution of the ketoester of general formula IV (1 equivalent) in ethanol was added the aldehyde of general formula V (1 equivalent), the carboxamidine hydrochloride of general formula VI (1 equivalent) and sodium acetate (1-1.2 equivalents). The mixture was brought up to 60-100° C. and stirred under nitrogen atmosphere overnight. After cooled down to room temperature, it was concentrated to dryness. The residue was taken up in dichloromethane, washed with water, brine, dried over anhydrous Na₂SO₄ and filtered. The filtrate was concentrated under reduced pressure to give a residue, which was purified by silica gel column chromatography to afford the dihydropyrimidine product of general formula VII. When applicable, the stereoisomers of the dihydropyrimidine product of general formula VII were isolated and purified using chiral chromatography to give the product of general formula VIIa and general formula of VIIb.

Method D:

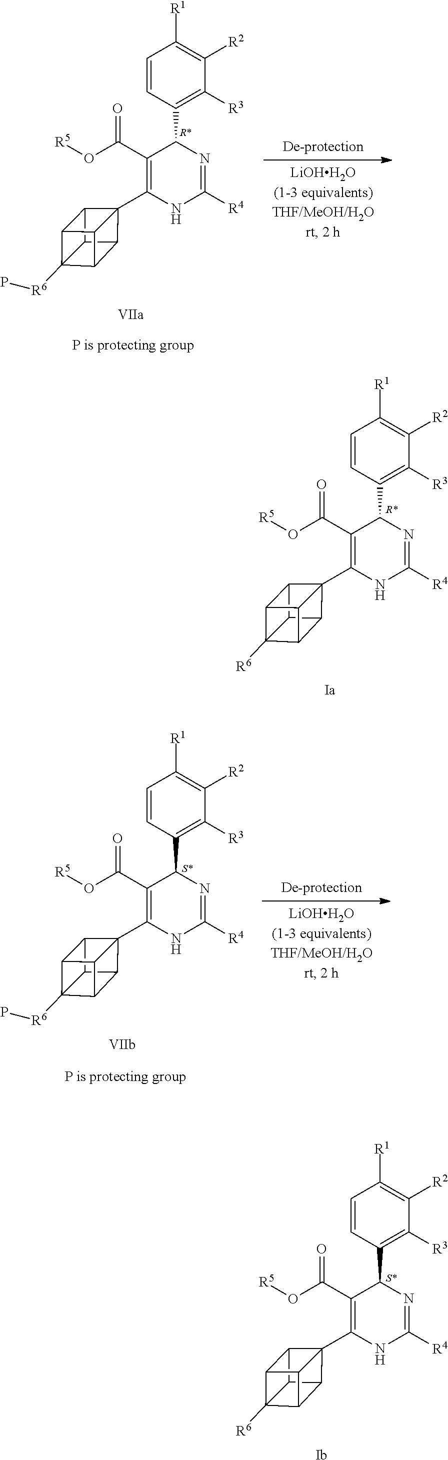

To a solution of the protected dihydropyrimidine product of general formula VIIa (1 equivalent), or formula VIIb (1 equivalent), in the solvents of tetrahydrofuran:methanol water 2:2:1 was added lithium hydroxide hydrate (1-3 equivalents) at 0° C. After stirred at 0° C. for 2 hours, the mixture was added with water, and concentrated at room temperature under reduced pressure to remove volatiles. The residue was acidified with 1 M hydrochloride aqueous solution and purified by silica gel column chromatography to afford the final compound of general formula Ta, or Ib, respectively. When applicable, the stereoisomers of the dihydropyrimidine product of general formula Ta and Ib were isolated and purified using chiral chromatography.

Chemistry

Several methods for preparing the compounds of this invention are illustrated hereinbelow. Unless otherwise noted, all starting materials were obtained from commercial suppliers and used without further purification.

Hereinafter, ACN means acetonitrile, AcOH means acetic acid, Boc means tert-butyloxycarbonyl, Bn means benzyl, calcd. means calculated, Cbz means benzyloxycarbonyl, CDI means N,N-carbonyldiimidazole, col. means column, conc. means concentrated, m-CPBA means 3-chloroperbenzoic acid, DABCO means 1,4-diazabicyclo[2.2.2]octane, DAST means (diethylamino)sulfur trifluoride, DCM means dichloromethane, DCE means dichloroethane, DEA means diethanolamine, DIEA means N,N-diisopropylethyl amine, DMAP means 4-(dimethylamino)pyridine, DMF means dimethylformamide, DMP means Dess-Martin periodinane, EA means ethyl acetate, ee means enantiomeric excess, ESI means electrospray ionization, HATU means 2-(7-azabenzotriazol-1-yl)-N,N,N′,N′-tetramethyluronium hexafluorophosphate, Hex means hexane, HNMR means ¹H NMR, HPLC means high performance liquid chromatography, IPA means isopropyl alcohol, LC-MS or LCMS means liquid chromatography-mass spectrometry, LDA means lithium diisopropylamide, Ms means methanesulfonyl, NBS means N-bromosuccinimide, NCS means N-chlorosuccinimide, PE means petroleum ether, PMB means 4-methoxybenzyl, prep. means preparative, Prep-HPLC means preparative HPLC, R_{T} or Rt mean retention time, ₍ₛ₎ or (s) mean solid, sat. means saturated, TBAF means tetrabutylammonium fluoride, TBS means tert-butyldimethylsilyl, TEA means triethylamine, THE means tetrahydrofuran, T or Temp mean temperature, TMSCH₂N₂ means (trimethylsilyl)diazomethane, TsCl means 4-toluenesulfonyl chloride, t-BuOK means potassium tert-butoxide, W means wavelength.

### Compound Ib-1: 2-((1S,2R,3R,8S)-4-((S*)-6-(2-chloro-4-fluorophenyl)-5-(methoxycarbonyl)-2-(thiazol-2-yl)-3,6-dihydropyrimidin-4-yl)cuban-1-yl)acetic Acid (a Single Stereoisomer)

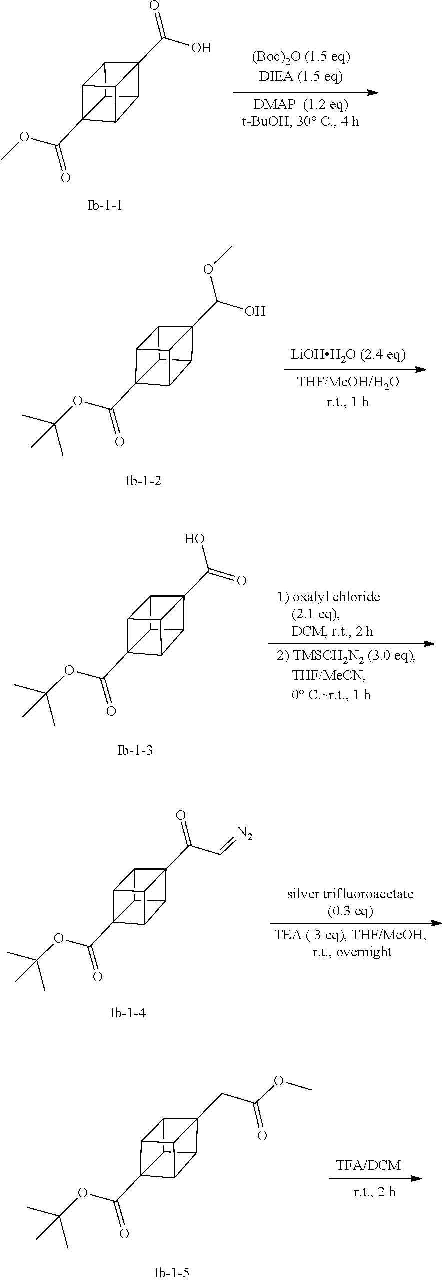

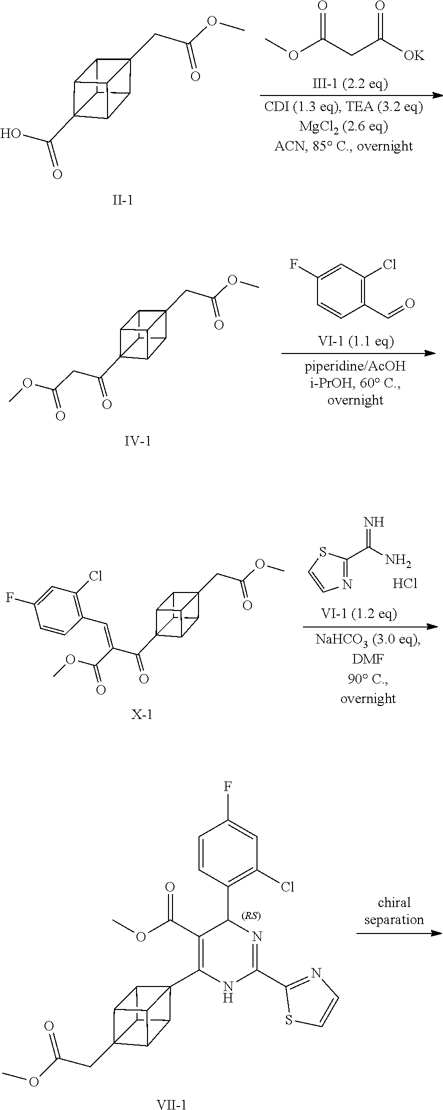

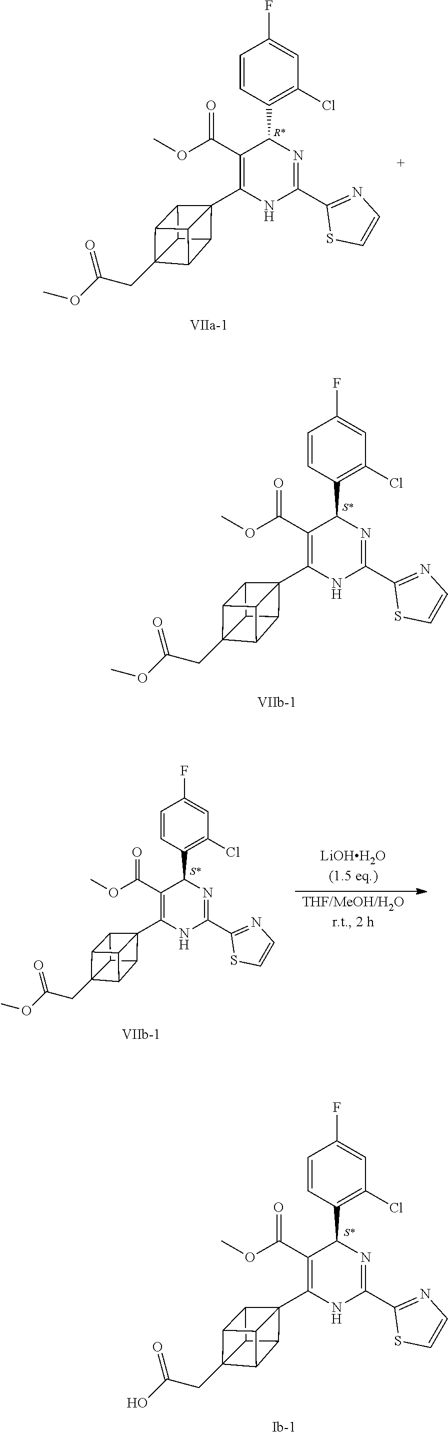

### Intermediate Ib-1-2: (1r,2R,3r,8S)-1-tert-butyl 4-methyl Cubane-1,4-dicarboxylate

To a solution of (2r,3R,4r,5S)-4-(methoxycarbonyl)cubane-1-carboxylic acid Ib-1-1 (500 mg, 2.43 mmol) in tert-butanol (10 mL) was added N,N-diisopropylethylamine (470 mg, 3.64 mmol), 4-dimethylaminopyridine (360 mg, 2.95 mmol) and di-tert-butyl dicarbonate (800 mg, 3.67 mmol). After stirred at 30° C. for 4 hours, the mixture was diluted with water (10 mL), acidified with 1 M hydrochloride aqueous solution to pH=4˜ 5 and then extracted with ethyl acetate (20 mL) twice. The combined organic layers were washed with brine (20 mL), dried over Na₂SO₄₍ₛ₎ and filtered. The filtrate was concentrated under reduced pressure to give the title compound (690 mg, 90% purity from ¹H NMR, 98% yield) as yellow solid. ¹H NMR (400 MHz, CDCl₃) δ 4.21-4.16 (m, 6H), 3.71 (s, 3H), 1.47 (s, 9H).

### Intermediate Ib-1-3: (2r,3R,4r,5S)-4-(tert-butoxycarbonyl)cubane-1-carboxylic Acid

To a solution of (1r,2R,3r,8S)-1-tert-butyl 4-methyl cubane-1,4-dicarboxylate Ib-1-2 (690 mg, 90% purity, 2.37 mmol) in methanol (3 mL), tetrahydrofuran (3 mL) and water (1 mL) was added lithium hydroxide monohydrate (240 mg, 5.720 mmol). After stirred at room temperature for 1 hour, the mixture was diluted with water (10 mL), acidified with 1 M hydrochloride aqueous solution to pH=4˜5 and then extracted with ethyl acetate (20 mL) twice. The combined organic layers were washed with brine (20 mL), dried over Na₂SO₄(s) and filtered. The filtrate was concentrated under reduced pressure to give the title compound (560 mg, 90% purity from ¹H NMR, 86% yield) as yellow solid. ¹H NMR (400 MHz, CDCl₃) δ 4.24-4.23 (m, 3H), 4.20-4.19 (m, 3H), 1.47 (s, 9H).

### Intermediate Ib-1-4: (1r,2R,3r,8S)-tert-butyl 4-(2-diazoacetyl)cubane-1-carboxylate

To the solution of (2r,3R,4r,5S)-4-(tert-butoxycarbonyl)cubane-1-carboxylic acid Ib-1-3 (550 mg, 90% purity, 1.99 mmol) in dichloromethane (10 mL) was added oxalyl chloride (0.35 mL, 4.14 mmol) and one drop of N,N-dimethylformamide at 0° C. After stirred at room temperature under nitrogen atmosphere for 2 hours, the mixture was concentrated and azeotroped with toluene to give brown oil, which was dissolved in dry tetrahydrofuran (10 mL) and acetonitrile (10 mL). To the resulting solution was added 2 M (trimethylsilyl)diazomethane in hexane (3 mL, 6.0 mmol) at 0° C. After stirred at room temperature for 1 hour under nitrogen atmosphere, the mixture was quenched with acetic acid (2 mL) and water (20 mL) at 0° C. and then extracted with ethyl acetate (20 mL) twice. The combined organic layers were washed with saturated sodium bicarbonate aqueous solution (20 mL) twice and brine (20 mL), dried over Na₂SO₄₍ₛ₎ and filtered. The filtrate was concentrated under reduced pressure to give the title compound (570 mg, 80% purity from ¹H NMR, 84% yield) as yellow solids. ¹H NMR (400 MHz, DMSO-d₆) δ 6.16 (s, 1H), 4.14-4.06 (m, 6H), 1.40 (s, 9H).

### Intermediate Ib-1-5: (1r,2R,3r,8S)-tert-butyl 4-(2-methoxy-2-oxoethyl)cubane-1-carboxylate

To a solution of silver trifluoroacetate (108 mg, 0.489 mmol) and triethylamine (495 mg, 4.89 mmol) in tetrahydrofuran (20 mL) and methanol (5 mL) was added a solution of (1r,2R,3r,8S)-tert-butyl 4-(2-diazoacetyl)cubane-1-carboxylate Ib-1-4 (550 mg, 80% purity, 1.62 mmol) in tetrahydrofuran (5 mL) dropwise at room temperature over 20 minutes. After stirred at room temperature overnight, the mixture was concentrated under reduced pressure to remove the volatile. The residue was diluted with water (30 mL) and extracted with ethyl acetate (30 mL) three times. The combined organic layers were washed with brine (30 mL), dried over Na₂SO₄₍ₛ₎ and filtered. The filtrate was concentrated and purified by silica gel column chromatography (petroleum ether:ethyl acetate=12:1 to 7:1) to give the title compound (440 mg, 80% purity from ¹H NMR, 79% yield) as light yellow oil. ¹H NMR (400 MHz, CDCl₃) δ 4.08-4.07 (m, 3H), 3.80-3.79 (m, 3H), 3.69-3.68 (m, 3H), 2.68-2.67 (m, 2H), 1.47 (s, 9H).

### Intermediate II-1: (1r,2R,3r,8S)-4-(2-methoxy-2-oxoethyl)cubane-1-carboxylic Acid

To a solution of (1r,2R,3r,8S)-tert-butyl 4-(2-methoxy-2-oxoethyl)cubane-1-carboxylate Ib-1-5 (440 mg, 80% purity, 1.27 mmol) in dichloromethane (10 mL) was added trifluoroacetic acid (5 mL) dropwise at 0° C. After stirred at room temperature for 2 hours, the mixture was concentrated under reduced pressure to give the title compound (350 mg, 70% purity from ¹H NMR, 87% yield) as yellow solid. ¹H NMR (400 MHz, CDCl₃) δ 4.20 (t, J=4.8 Hz, 3H), 3.86 (t, J=4.4 Hz, 3H), 3.67 (s, 3H), 2.69 (s, 2H).

### Intermediate IV-1: Methyl 3-((1r,2R,3r,8S)-4-(2-methoxy-2-oxoethyl)cuban-1-yl)-3-oxopropanoate (Exemplified with Method A)

To the solution of (1r,2R,3r,8S)-4-(2-methoxy-2-oxoethyl)cubane-1-carboxylic acid IT-1 (350 mg, 70% purity, 1.11 mmol) in acetonitrile (5 mL) was added 1,1′-carbonyldiimidazole (227.5 mg, 1.40 mmol) at room temperature. The solution was stirred at room temperature under nitrogen atmosphere for 1 hour (mixture A). To the suspension of methyl potassium malonate III-1 (376 mg, 2.41 mmol) and magnesium chloride (271 mg, 2.85 mmol) in acetonitrile (10 mL) was added triethylamine (360 mg, 3.56 mmol). After stirred at room temperature under nitrogen atmosphere for 1 hour, the suspension was added mixture A and stirring continued at 85° C. under nitrogen atmosphere overnight. Then it was cooled down and concentrated under reduced pressure to give a residue, which was diluted with water (15 mL) and ethyl acetate (20 mL). The mixture was acidified with potassium bisulfate(s) until pH ˜3 and then the organic layer was separated. The aqueous layer was extracted with ethyl acetate (20 mL) twice. The combined organic layers were washed with saturated sodium bicarbonate aqueous solution (20 mL) and brine (20 mL), dried over Na₂SO₄₍ₛ₎ and filtered. The filtrate was concentrated under reduced pressure to give the title compound (120 mg, 80% purity from ¹H NMR, 31% yield) as yellow oil which was directly used in next step without further purification. ¹H NMR (400 MHz, CDCl₃) δ 11.80 (s, 0.2H), 4.59 (s, 0.2H), 4.23 (t, J=4.8 Hz, 3H), 3.83 (t, J=4.8 Hz, 3H), 3.74 (s, 3H), 3.69 (s, 3H), 3.49 (s, 1.6H), 2.69 (s, 2H).

### Intermediate X-1: Methyl 3-(2-chloro-4-fluorophenyl)-2-((1r,2R,3r,8S)-4-(2-methoxy-2-oxoethyl)cubane-1-carbonyl)acrylate (Exemplified with Method B Step 1)

To a solution of methyl 3-((1r,2R,3r,8S)-4-(2-methoxy-2-oxoethyl)cuban-1-yl)-3-oxopropanoate IV-1 (120 mg, 80% purity, 0.347 mmol) in isopropyl alcohol (10 mL) was added 2-chloro-4-fluorobenzaldehyde V-1 (65 mg, 0.410 mmol) and piperidine (0.1 mL) and acetic acid (0.1 mL) at room temperature. After stirred at 60° C. under nitrogen atmosphere overnight, the mixture was cooled down to room temperature, concentrated and purified by silica gel column chromatography (petroleum ether:ethyl acetate=5:1) to give the title compound (120 mg, 90% purity from ¹H NMR, 75% yield) as yellow oil. ¹H NMR (400 MHz, CDCl₃) δ 7.95 (s, 0.4H), 7.70 (s, 0.6H), 7.45 (dd, J=9.2 Hz, 6.0 Hz, 0.6H), 7.31 (dd, J=8.8 Hz, 6.0 Hz, 0.4H), 7.22-7.17 (m, 1H), 7.02-6.92 (m, 1H), 4.36 (t, J=4.8 Hz, 1.8H), 3.94-3.90 (m, 3H), 3.85 (s, 1.2H), 3.77 (s, 1.8H), 3.70 (s, 1.8H), 3.68-3.65 (m, 2.4H), 2.72 (s, 1.2H), 2.61 (s, 0.8H).

### Intermediate VII-1: Methyl 4-(2-chloro-4-fluorophenyl)-6-((2r,3R,4s,5S)-4-(2-methoxy-2-oxoethyl)cuban-1-yl)-2-(thiazol-2-yl)-1,4-dihydropyrimidine-5-carboxylate (a Mixture of 2 Stereoisomers) (Exemplified with Method B Step 2)

To a solution of methyl 3-(2-chloro-4-fluorophenyl)-2-((1r,2R,3r,8S)-4-(2-methoxy-2-oxoethyl)cubane-1-carbonyl)acrylate X-1 (120 mg, 90% purity, 0.259 mmol) in N,N-dimethylformamide (2 mL) was added thiazole-2-carboximidamide hydrochloride VI-1 (51 mg, 0.312 mmol) and sodium hydrogencarbonate (65 mg, 0.774 mmol) at room temperature. After stirred at 90° C. under nitrogen atmosphere overnight, the mixture was cooled down to room temperature, poured into water (200 mL) and filtered to give a crude product, which was purified by silica gel column chromatography (petroleum ether:ethyl acetate=3:1) to give the title compound (85 mg, 95% purity from ¹H NMR, 59% yield) as yellow solids. ¹H NMR (400 MHz, CDCl₃) δ 8.08 (br s, 1H), 7.82 (d, J=2.8 Hz, 1H), 7.46 (br s, 1H), 7.31 (dd, J=8.4 Hz, 6.4 Hz, 1H), 7.13 (dd, J=8.8 Hz, 2.4 Hz, 1H), 6.94-6.89 (m, 1H), 6.20 (br s, 1H), 4.19 (br s, 3H), 3.91 (br s, 3H), 3.71 (s, 3H), 3.63 (s, 3H), 2.74 (s, 2H).

A racemic mixture of methyl 4-(2-chloro-4-fluorophenyl)-6-((2r,3R,4s,5S)-4-(2-methoxy-2-oxoethyl)cuban-1-yl)-2-(thiazol-2-yl)-1,4-dihydropyrimidine-5-carboxylate VII-1 (85 mg, 95% purity, 0.154 mmol) was separated by chiral Prep. HPLC (separation condition: column: Chiralpak IG 5 μm 20*250 mm; Mobile Phase: Hex:EtOH:DEA=50:50:0.3 at 15 mL/min, Temp: 30° C., Wavelength: 230 nm) to give (4R*)-methyl 4-(2-chloro-4-fluorophenyl)-6-((2R,3R,4S,5S)-4-(2-methoxy-2-oxoethyl)cuban-1-yl)-2-(thiazol-2-yl)-1,4-dihydropyrimidine-5-carboxylate VIIa-1 (40 mg, 90% purity from ¹H NMR, 45% yield, 100% ee) and (4S*)-methyl 4-(2-chloro-4-fluorophenyl)-6-((2R,3R,4S,5S)-4-(2-methoxy-2-oxoethyl)cuban-1-yl)-2-(thiazol-2-yl)-1,4-dihydropyrimidine-5-carboxylate VIIb-1 (35 mg, 90% purity from ¹H NMR, 39% yield, 99.8% ee) as yellow solids.

Intermediate VIIa-1 (a single stereoisomer): Chiral analysis (Column: Chiralpak IG 5 um 4.6*250 mm; Mobile Phase: Hex:EtOH:DEA=50:50:0.2 at 1.0 mL/min; Temp: 30° C.; Wavelength: 254 nm, R_{T}=7.015 min). ¹H NMR (400 MHz, CDCl₃) δ 8.08 (br s, 1H), 7.82 (d, J=2.8 Hz, 1H), 7.46 (d, J=2.8 Hz, 1H), 7.33-7.30 (m, 1H), 7.13 (dd, J=8.8 Hz, 2.4 Hz, 1H), 6.94-6.89 (m, 1H), 6.18 (br s, 1H), 4.19 (t, J=4.4 Hz, 3H), 3.91 (t, J=4.4 Hz, 3H), 3.71 (s, 3H), 3.63 (s, 3H), 2.74 (s, 2H).

Intermediate VIIb-1 (a single stereoisomer): Chiral analysis (Column: Chiralpak IG 5 um 4.6*250 mm; Mobile Phase: Hex:EtOH:DEA=50:50: 0.2 at 1.0 mL/min; Temp: 30° C.; Wavelength: 254 nm, R_{T}=12.088 min). ¹H NMR (400 MHz, CDCl₃) δ 8.05 (br s, 1H), 7.82 (d, J=3.2 Hz, 1H), 7.45 (d, J=2.4 Hz, 1H), 7.33-7.29 (m, 1H), 7.13 (dd, J=8.4 Hz, 2.4 Hz, 1H), 6.94-6.89 (m, 1H), 6.18 (br s, 1H), 4.19 (t, J=4.8 Hz, 3H), 3.91 (t, J=5.2 Hz, 3H), 3.71 (s, 3H), 3.63 (s, 3H), 2.74 (s, 2H).

### Compound Ib-1: 2-((1S,2R,3R,8S)-4-((S*)-6-(2-chloro-4-fluorophenyl)-5-(methoxycarbonyl)-2-(thiazol-2-yl)-3,6-dihydropyrimidin-4-yl)cuban-1-yl)acetic Acid (a Single Stereoisomer) (Exemplified with Method D)

To a solution of (4S*)-methyl 4-(2-chloro-4-fluorophenyl)-6-((2R,3R,4S,5S)-4-(2-methoxy-2-oxoethyl)cuban-1-yl)-2-(thiazol-2-yl)-1,4-dihydropyrimidine-5-carboxylate VIIb-1 (35 mg, 90% purity, 0.060 mmol) in tetrahydrofuran (1.5 mL), water (0.5 mL) and methanol (0.5 mL) was added lithium hydroxide monohydrate (4.0 mg, 0.095 mmol) under nitrogen atmosphere.

After stirred at room temperature for 2 hours, the mixture was poured into water (5 mL), acidified by 0.5 M hydrochloride aqueous solution to pH ˜5 and then extracted with ethyl acetate (5 mL) twice. The combined organic layers were washed with water (5 mL) and brine (5 mL), dried over Na₂SO₄₍ₛ₎ and filtered. The filtrate was concentrated and purified by C18 column (acetonitrile:water=10% to 80%) to give the title compound (20.2 mg, 98.9% purity, 65% yield, 99.5% ee) as yellow solid. LC-MS (ESI): mass calcd. for C₂₅H₁₉ClFN₃O₄S 511.1, m/z found 512.1 [M+H]⁺. Chiral analysis (Column: Chiralpak IC 5 um 4.6*250 mm; Mobile Phase: Hex:EtOH:TFA=70:30:0.2 at 1.0 mL/min; Temp: 30° C.; Wavelength: 254 nm, R_{T}=6.112 min). ¹H NMR (400 MHz, CDCl₃) δ 8.06 (br s, 1H), 7.81-7.80 (m, 1H), 7.46-7.43 (m, 1H), 7.31 (dd, J=8.4 Hz, 6.0 Hz, 1H), 7.12 (dd, J=8.4 Hz, 2.4 Hz, 1H), 6.93-6.89 (m, 1H), 6.21 (s, 0.8H), 6.08 (br s, 0.2H), 4.24-4.20 (m, 3H), 3.96-3.91 (m, 3H), 3.66-3.62 (m, 3H), 2.78 (s, 2H).

### Compound Ib-2: 2-((1R,2R,3R,8S)-4-((S*)-6-(2-chloro-4-fluoro-phenyl)-5-(methoxycarbonyl)-2-(thiazol-2-yl)-3,6-dihydropyrimidin-4-yl)cuban-1-yl)oxazole-4-carboxylic Acid (a Single Stereoisomer)

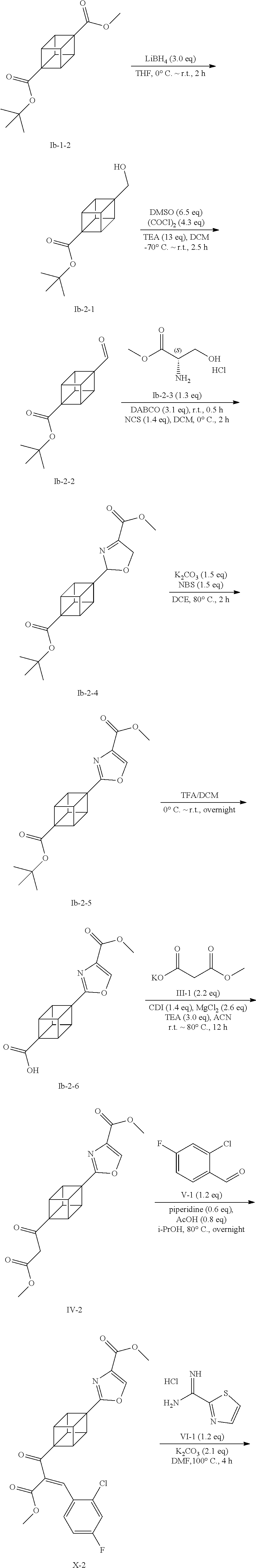

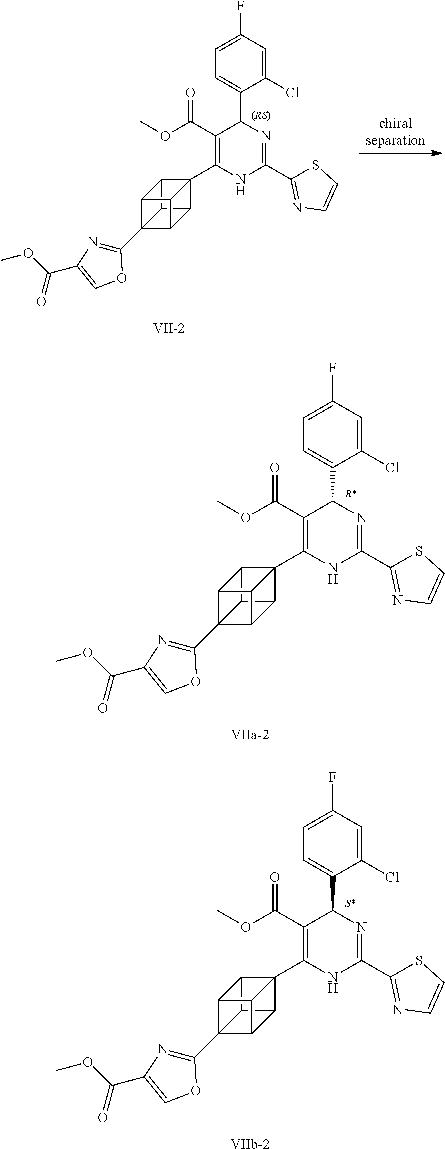

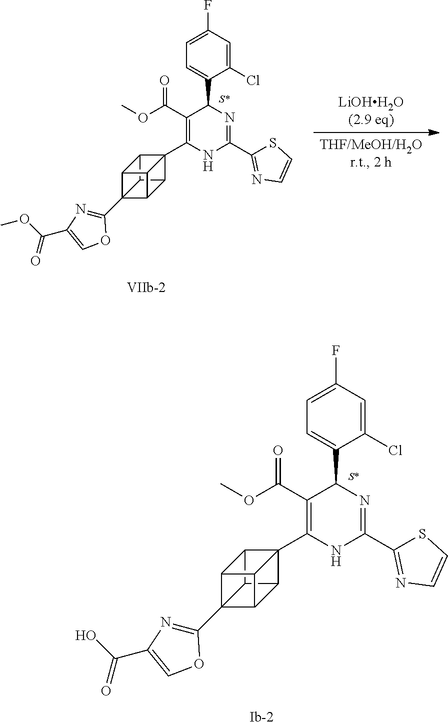 [SMILES: COC(=O)C1=C(C23C4C5C2C2C3C4C52c2nc(C(=O)OC)co2)NC(c2nccs2)=N[C@@H]1c1ccc(F)cc1Cl ; "VIIb-2" ; COC(=O)C1=C(C23C4C5C2C2C3C4C52c2nc(C(=O)O)co2)NC(c2nccs2)=N[C@@H]1c1ccc(F)cc1Cl ; "Ib-2"]

### Intermediate Ib-2-1: (1r,2R,3r,8S)-tert-butyl 4-(hydroxymethyl)cubane-1-carboxylate

To a solution of (1r,2R,3r,8S)-1-tert-butyl 4-methyl cubane-1,4-dicarboxylate Ib-1-2 (1.5 g, 90% purity, 5.15 mmol) in anhydrous tetrahydrofuran (10 ml) was added lithium borohydride (336 mg, 15.4 mmol) slowly at 0° C. After stirred at room temperature for 2 hours, the reaction mixture was quenched with saturated ammonium chloride aqueous solution (30 ml) and extracted with ethyl acetate (20 mL) twice. The combined organic layers were washed with brine (20 mL), dried over Na₂SO₄₍ₛ₎ and filtered. The filtrate was concentrated and purified by C18 column (acetonitrile:water=20% to 75%) to give the title compound (1.3 g, 90% purity from ¹H NMR, 97% yield) as white solid. ¹H NMR (400 MHz, CDCl₃) δ 4.09-4.07 (m, 3H), 3.86-3.84 (m, 3H), 3.77 (s, 2H), 1.47 (s, 9H).

### Intermediate Ib-2-2: (1r,2R,3r,8S)-tert-butyl 4-formylcubane-1-carboxylate

To a solution of anhydrous dimethyl sulfoxide (2.52 g, 32.3 mmol) in anhydrous dichloromethane (15 mL) was added oxalyl dichloride (2.72 g, 21.4 mmol) dropwise at −70° C. The mixture was stirred at −70° C. under nitrogen atmosphere for 1 hour and then a solution of (1r,2R,3r,8S)-tert-butyl 4-(hydroxymethyl)cubane-1-carboxylate Ib-2-1 (1.3 g, 90% purity, 4.99 mmol) in anhydrous dichloromethane (5 mL) was added dropwise. The mixture was stirred at −70° C. for another 1 hour and then triethylamine (6.431 g, 63.6 mmol) was added. After stirred at room temperature for 0.5 hour, the reaction mixture was diluted with ice water (30 mL), acidified with 0.5 M hydrochloride aqueous solution to pH=6˜7 and then extracted with dichloromethane (10 mL) three times. The combined organic layers were washed with saturated sodium bicarbonate aqueous solution (10 mL) and brine (10 mL) three times, dried over Na₂SO₄₍ₛ₎ and filtered. The filtrate was concentrated under reduced pressure to give the title compound (1.3 g, 85% purity from ¹H NMR, 95% yield) as light-yellow solid. ¹H NMR (400 MHz, CDCl₃) δ 9.75 (s, 1H), 4.35-4.33 (m, 3H), 4.20-4.18 (m, 3H), 1.47 (s, 9H).

### Intermediate Ib-2-4: Methyl 2-((2r,3R,4r,5S)-4-(tert-butoxycarbonyl)cuban-1-yl)-2,5-dihydrooxazole-4-carboxylate

To a mixture of (S)-methyl 2-amino-3-hydroxypropanoate hydrochloride Ib-2-3 (992 mg, 95% purity, 6.06 mmol) in dichloromethane (10 mL) was added 1,4-diazabicyclo[2.2.2]octane (1.671 g, 14.9 mmol). The mixture was stirred at room temperature under nitrogen atmosphere for 30 minutes and then a solution of (1r,2R,3r,8S)-tert-butyl 4-formylcubane-1-carboxylate Ib-2-2 (1.3 g, 85% purity, 4.76 mmol) in dichloromethane (5 mL) was added. The mixture was stirred at 25° C. under nitrogen atmosphere for another 30 minutes and then N-chlorosuccinimide (874 mg, 6.55 mmol) was added at 0° C. After stirred at 0° C. for 2 hours, the reaction mixture was quenched with saturated sodium metabisulfite aqueous solution (10 mL) at the same temperature. The organic layer was separated and the aqueous layer was extracted with dichloromethane (50 mL) twice. The combined organic layers were dried over Na₂SO₄₍ₛ₎ and filtered. The filtrate was concentrated and purified by silica gel column chromatography (petroleum ether:ethyl acetate=10:1 to 5:1) to give the title compound (1 g, 90% purity from ¹H NMR, 57% yield) as colorless oil. ¹H NMR (400 MHz, CDCl₃) δ 6.08-6.06 (m, 1H), 4.90-4.77 (m, 2H), 4.11-4.08 (m, 3H), 3.96-3.94 (m, 6H), 1.46 (s, 9H).

### Intermediate Ib-2-5: Methyl 2-((2r,3R,4r,5S)-4-(tert-butoxycarbonyl)cuban-1-yl)oxazole-4-carboxylate

A mixture of methyl 2-((2r,3R,4r,5S)-4-(tert-butoxycarbonyl)cuban-1-yl)-2,5-dihydrooxazole-4-carboxylate Ib-2-4 (900 mg, 90% purity, 2.44 mmol), potassium carbonate (506 mg, 3.67 mmol) and 4 Å molecular sieves (1 g) in 1,2-dichloroethane (5 mL) was stirred at room temperature under nitrogen atmosphere for 2 hours and then N-bromosuccinimide (0.653 g, 3.667 mmol) was added. After stirred at 80° C. for 2 hours, the reaction mixture was cooled down to room temperature and quenched with saturated sodium sulfite aqueous solution (25 mL) and saturated sodium bicarbonate aqueous solution (25 mL). The organic layer was separated and the aqueous layer was extracted with dichloromethane (50 mL) twice. The combined organic layers were dried over Na₂SO₄₍ₛ₎ and filtered. The filtrate was concentrated and purified by silica gel column chromatography (petroleum ether:ethyl acetate=4:1) to give the title compound (450 mg, 95.5% purity, 53% yield) as white solid. LC-MS (ESI): mass calcd. for C₁₈H₁₉NO₅ 329.1, m/z found 330.4 [M+H]⁺. ¹H NMR (400 MHz, CDCl₃) δ 8.20 (s, 1H), 4.37-4.35 (m, 3H), 4.26-4.23 (m, 3H), 3.92 (s, 3H), 1.48 (s, 9H).

### Intermediate Ib-2-6: (1r,2R,3r,8S)-4-(4-(methoxycarbonyl)oxazol-2-yl)cubane-1-carboxylic Acid

To a solution of methyl 2-((2r,3R,4r,5S)-4-(tert-butoxycarbonyl)cuban-1-yl) oxazole-4-carboxylate Ib-2-5 (300 mg, 95.5% purity, 0.870 mmol) in dichloromethane (5 mL) was added trifluoroacetic acid (5 mL) at 0° C. After stirred at room temperature overnight, the reaction mixture was concentrated and purified by C18 column (acetonitrile:water=20% to 70%) to give the title compund (260 mg, 87.6% purity, 96% yield) as white solids. LC-MS (ESI): mass calcd. for C₁₄H₁₁NO₅ 273.1, m/z found 274.1 [M+H]⁺. ¹H NMR (400 MHz, CDCl₃) δ 8.21 (s, 1H), 4.44-4.42 (m, 3H), 4.38-4.35 (m, 3H), 3.92 (s, 3H).

### Intermediate IV-2: Methyl 2-((2r,3R,4r,5S)-4-(3-methoxy-3-oxopropanoyl)cuban-1-yl)oxazole-4-carboxylate

Converted from compounds Ib-2-6 and III-1.

By utilizing the analogous procedure of Method A, the title compound was synthesized as brown oil. LC-MS (ESI): mass calcd. for C₁₇H₁₅NO₆ 329.1, m/z found 330.1 [M+H]⁺. ¹H NMR (400 MHz, CDCl₃) δ 8.21 (s, 1H), 4.39 (s, 6H), 3.92 (s, 3H), 3.75 (s, 3H), 3.52 (s, 2H).

### Intermediate X-2: Methyl 2-((2r,3R,4r,5S)-4-(3-(2-chloro-4-fluorophenyl)-2-(methoxycarbonyl)acryloyl)cuban-1-yl)oxazole-4-carboxylate

Converted from compounds IV-2 and V-1.

By utilizing the analogous procedure of Method B step 1, the title compound was synthesized as yellow oil. LC-MS (ESI): mass calcd. for C₂₄H₁₇ClFNO₆ 469.1, m/z found 470.1 [M+H]⁺.

### Intermediate VII-2: Methyl 2-((1r,2R,3r,8S)-4-(6-(2-chloro-4-fluorophenyl)-5-(methoxycarbonyl)-2-(thiazol-2-yl)-3,6-dihydropyrimidin-4-yl)cuban-1-yl)oxazole-4-carboxylate (a Mixture of 2 Stereoisomers)

Converted from compounds X-2 and VI-1.

By utilizing the analogous procedure of Method B step 2, the title compound was synthesized as yellow solids. LC-MS (ESI): mass calcd. for C₂₈H₂₀ClFN₄O₅S 578.1, m/z found 579.1 [M+H]⁺. ¹H NMR (400 MHz, CDCl₃) δ 8.23 (s, 1H), 8.05 (s, 0.5H), 7.85-7.82 (m, 1H), 7.51 (d, J=3.2 Hz, 1H), 7.45 (d, J=3.2 Hz, 0.5H), 7.33-7.30 (m, 1H), 7.16-7.13 (m, 1H), 6.97-6.90 (m, 1H), 6.21 (s, 0.5H), 6.09 (d, J=2.4 Hz, 0.5H), 4.48-4.32 (m, 6H), 3.94 (s, 3H), 3.67 (s, 1.2H), 3.63 (s, 1.8H).

A racemic of methyl 2-((1r,2R,3r,8S)-4-(6-(2-chloro-4-fluorophenyl)-5-(methoxycarbonyl)-2-(thiazol-2-yl)-3,6-dihydropyrimidin-4-yl)cuban-1-yl)oxazole-4-carboxylate VII-2 (220 mg, 96.8% purity, 0.368 mmol) was separated by chiral Prep. HPLC (separation condition: Column: Chiralpak IE 5 um*20*250 mm; Mobile Phase: Hex:EtOH=60:40 at 10 mL/min; Col. Temp: 35° C.; Wavelength: 254 nm) to give methyl 2-((1R,2R,3R,8S)-4-((R*)-6-(2-chloro-4-fluorophenyl)-5-(methoxycarbonyl)-2-(thiazol-2-yl)-3,6-dihydropyrimidin-4-yl)cuban-1-yl)oxazole-4-carboxylate VIIa-2 (95 mg, 95% purity from ¹H NMR, 42% yield, 100% ee) and methyl 2-((1R,2R,3R,8S)-4-((S*)-6-(2-chloro-4-fluorophenyl)-5-(methoxycarbonyl)-2-(thiazol-2-yl)-3,6-dihydropyrimidin-4-yl)cuban-1-yl)oxazole-4-carboxylate VIIb-2 (95 mg, 95% purity from ¹H NMR, 42% yield, 99.4% ee) as yellow solids.

Intermediate VIIa-2 (a single stereoisomer): LC-MS (ESI): mass calcd. for C₂₈H₂₀ClFN₄O₅S 578.1, m/z found 579.1 [M+H]⁺. Chiral analysis (Column: Chiralpak IE 5 um 4.6*250 mm; Mobile Phase: Hex:EtOH=60:40 at 1 mL/min; Temp: 30° C.; Wavelength: 254 nm, R_{T}=11.146 min). ¹H NMR (400 MHz, CDCl₃) δ 8.23 (s, 1H), 8.05 (s, 0.5H), 7.85-7.82 (m, 1H), 7.51 (d, J=3.2 Hz, 1H), 7.45 (d, J=3.2 Hz, 0.5H), 7.33-7.29 (m, 1H), 7.15-7.12 (m, 1H), 7.00-6.90 (m, 1H), 6.21 (s, 0.5H), 6.09 (d, J=2.4 Hz, 0.5H), 4.49-4.32 (m, 6H), 3.94 (s, 3H), 3.67 (s, 1.2H), 3.63 (s, 1.8H).

Intermediate VIIb-2 (a single stereoisomer): LC-MS (ESI): mass calcd. for C₂₈H₂₀ClFN₄O₅S 578.1, m/z found 579.1 [M+H]⁺. Chiral analysis (Column: Chiralpak IE 5 um 4.6*250 mm; Mobile Phase: Hex:EtOH=60:40 at 1 mL/min; Temp: 30° C.; Wavelength: 254 nm; R_{T}=13.009 min). ¹H NMR (400 MHz, CDCl₃) δ 8.23 (s, 1H), 8.05 (s, 0.5H), 7.85-7.83 (m, 1H), 7.51 (d, J=3.2 Hz, 1H), 7.45 (d, J=3.2 Hz, 0.5H), 7.33-7.29 (m, 1H), 7.15-7.13 (m, 1H), 6.98-6.90 (m, 1H), 6.21 (s, 0.5H), 6.09 (d, J=2.4 Hz, 0.5H), 4.49-4.32 (m, 6H), 3.94 (s, 3H), 3.67 (s, 1.2H), 3.63 (s, 1.8H).

### Compound Ib-2: 2-((1R,2R,3R,8S)-4-((S*)-6-(2-chloro-4-fluoro-phenyl)-5-(methoxycarbonyl)-2-(thiazol-2-yl)-3,6-dihydropyrimidin-4-yl)cuban-1-yl)oxazole-4-carboxylic Acid (a Single Stereoisomer)

LC-MS (ESI): mass calcd. for C₂₇H₁₈ClFN₄O₅S 564.1, m/z found 565.0 [M+H]⁺. ¹H NMR (400 MHz, DMSO-d₆) δ 8.40 (s, 1H), 8.01 (s, 1.5H), 7.94 (d, J=3.2 Hz, 0.5H), 7.44-7.38 (m, 2H), 7.24-7.20 (m, 1H), 6.03 (s, 0.4H), 5.95 (s, 0.6H), 4.32-4.28 (m, 6H), 3.59 (s, 3H).

### Compound Ib-3: (1R,2R,3R,8S)-4-((S*)-6-(2-chloro-4-fluorophenyl)-5-(methoxycarbonyl)-2-(thiazol-2-yl)-3,6-dihydropyrimidin-4-yl)cubane-1-carboxylic Acid (a Single Stereoisomer)

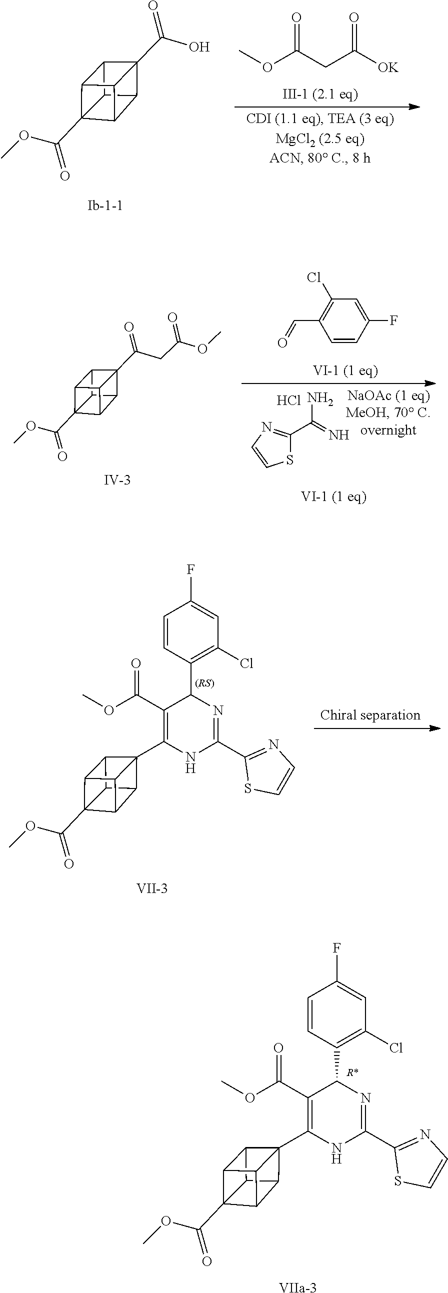

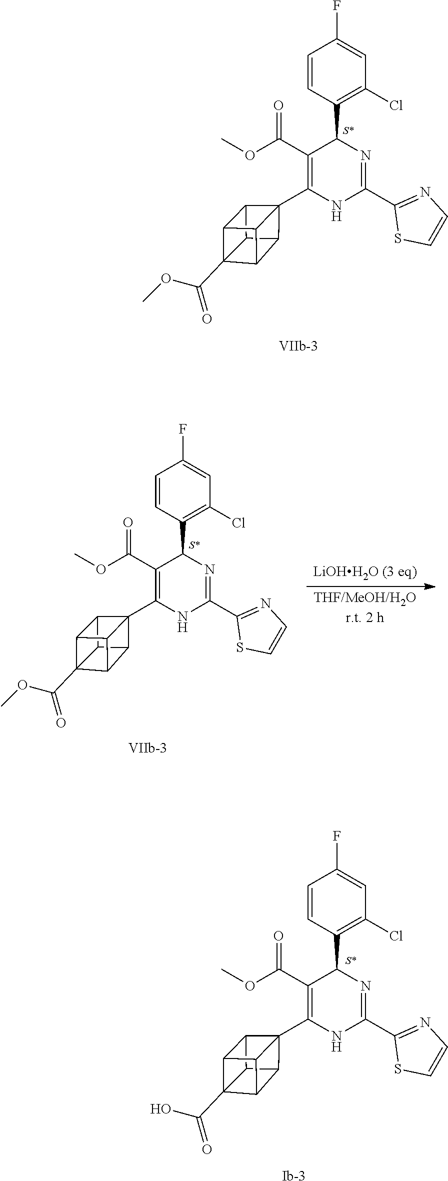 [SMILES: COC(=O)C1=C(C23C4C5C2C2C3C4C52C(=O)OC)NC(c2nccs2)=N[C@@H]1c1ccc(F)cc1Cl ; "VIIb-3" ; COC(=O)C1=C(C23C4C5C2C2C3C4C52C(=O)OC)NC(c2nccs2)=N[C@@H]1c1ccc(F)cc1Cl ; "VIIb-3" ; COC(=O)C1=C(C23C4C5C2C2C3C4C52C(=O)O)NC(c2nccs2)=N[C@@H]1c1ccc(F)cc1Cl ; "Ib-3"]

### Intermediate IV-3: (1r,2R,3r,8S)-methyl 4-(3-methoxy-3-oxopropanoyl)cubane-1-carboxylate

Converted from compounds Ib-1-1 and III-1.

By utilizing the analogous procedure of Method A, the title compound was synthesized as yellow solid. LC-MS (ESI): mass calcd. for C₁₄H₁₄O₅ 262.1, m/z found 263.0 [M+H]⁺. ¹H NMR (400 MHz, CDCl₃) δ 11.80 (s, 0.2H), 4.96 (s, 0.2H), 4.32-4.30 (m, 2H), 4.24-4.22 (m, 4H), 3.74 (s, 3H), 3.72 (s, 3H), 3.49 (s, 1.6H).

### Intermediate VII-3: Methyl 4-(2-chloro-4-fluorophenyl)-6-((2r,3R,4r,5S)-4-(methoxycarbonyl)cuban-1-yl)-2-(thiazol-2-yl)-1,4-dihydropyrimidine-5-carboxylate (a Mixture of 2 Stereoisomers) (Exemplified with Method C)

To a solution of (1r,2R,3r,8S)-methyl 4-(3-methoxy-3-oxopropanoyl)cubane-1-carboxylate IV-3 (1.1 g, 80% purity, 3.36 mmol), 2-chloro-4-fluorobenzaldehyde V-1 (533 mg, 3.36 mmol) and thiazole-2-carboximidamide hydrochloride VI-1 (549 mg, 3.36 mmol) in methanol (50 mL) was added sodium acetate (276 mg, 3.36 mmol) at room temperature. After stirred at 70° C. under nitrogen atmosphere overnight, the mixture was cooled down to room temperature, concentrated and purified by silica gel column chromatography (petroleum ether:ethyl acetate=10:1 to 6:1) to give a crude product, which was further purified by C18 column (acetonitrile:water=70% to 80%) to give the title compound (1.1 g, 90% purity from ¹H NMR, 58% yield) as yellow solids. LC-MS (ESI): mass calcd. for C₂₅H₁₉ClFN₃O₄S 511.1, m/z found 512.0 [M+H]⁺. ¹H NMR (400 MHz, CDCl₃) δ 8.03 (s, 0.6H), 7.84-7.81 (m, 1H), 7.51 (d, J=3.2 Hz, 0.8H), 7.44 (d, J=2.8 Hz, 0.6H), 7.32-7.28 (m, 1H), 7.15-7.12 (m, 1H), 6.95-6.90 (m, 1H), 6.20 (s, 0.6H), 6.08 (d, J=2.4 Hz, 0.4H), 4.34-4.30 (m, 3H), 4.28-4.24 (m, 3H), 3.74 (s, 3H), 3.66 (s, 1.2H), 3.62 (s, 1.8H).

A mixture of methyl 4-(2-chloro-4-fluorophenyl)-6-((2r,3R,4r,5S)-4-(methoxycarbonyl)cuban-1-yl)-2-(thiazol-2-yl)-1,4-dihydropyrimidine-5-carboxylate VII-3 (330 mg, 90% purity, 0.58 mmol) was separated by chiral Prep. SFC (the separation condition: Column: Chiralpak ID 5 um 20*250 mm; Mobile Phase: CO₂:EtOH=75:25 at 50 g/min; Col. Temp: 40° C.; Wavelength: 230 nm, Back pressure: 100 bar) to give (4R*)-methyl 4-(2-chloro-4-fluorophenyl)-6-((2R,3R,4R,5S)-4-(methoxycarbonyl)cuban-1-yl)-2-(thiazol-2-yl)-1,4-dihydropyrimidine-5-carboxylate VIIa-3 (140 mg, 90% purity from ¹H NMR, 42% yield, 99.2% stereopure) as yellow solids and (4S*)-methyl 4-(2-chloro-4-fluorophenyl)-6-((2R,3R,4R,5S)-4-(methoxycarbonyl)cuban-1-yl)-2-(thiazol-2-yl)-1,4-dihydropyrimidine-5-carboxylate VIIb-3 (130 mg, 90% purity from ¹H NMR, 39% yield, 93.4% stereopure) as yellow solids.

Intermediate VIIa-3 (a single stereoisomer): LC-MS (ESI): mass calcd. for C₂₅H₁₆ClFN₃O₄S 511.1, m/z found 512.0 [M+H]⁺. Chiral analysis (Column: Chiralpak ID 5 um 4.6*250 mm; Mobile Phase: CO₂:EtOH=75:25 at 3 g/min; Temp: 40° C.; Wavelength: 230 nm, R_{T}=5.63 min). ¹H NMR (400 MHz, CDCl₃) δ 8.02 (s, 0.6H), 7.84-7.81 (m, 1H), 7.52-7.50 (m, 0.8H), 7.44 (d, J=3.2 Hz, 0.6H), 7.32-7.28 (m, 1H), 7.15-7.12 (m, 1H), 6.97-6.89 (m, 1H), 6.20 (s, 0.6H), 6.08 (d, J=2.4 Hz, 0.4H), 4.34-4.30 (m, 3H), 4.27-4.24 (m, 3H), 3.74 (s, 3H), 3.66 (s, 1.2H), 3.62 (s, 1.8H).

Intermediate VIIb-3 (a single stereoisomer): LC-MS (ESI): mass calcd. for C₂₅H₁₆ClFN₃O₄S 511.1, m/z found 512.0 [M+H]⁺. Chiral analysis (Column: Chiralpak ID 5 um 4.6*250 mm; Mobile Phase: CO₂:EtOH=75:25 at 3 g/min; Temp: 40° C.; Wavelength: 230 nm, R_{T}=6.66 min). ¹H NMR (400 MHz, CDCl₃) δ 8.02 (s, 0.6H), 7.84-7.81 (m, 1H), 7.54-7.50 (m, 0.8H), 7.44 (d, J=2.8 Hz, 0.6H), 7.32-7.28 (m, 1H), 7.15-7.12 (m, 1H), 6.97-6.89 (m, 1H), 6.20 (s, 0.6H), 6.08 (d, J=2.0 Hz, 0.4H), 4.34-4.30 (m, 3H), 4.28-4.24 (m, 3H), 3.74 (s, 3H), 3.66 (s, 1.2H), 3.62 (s, 1.8H).

### Compound Ib-3: (1R,2R,3R,8S)-4-((S*)-6-(2-chloro-4-fluorophenyl)-5-(methoxycarbonyl)-2-(thiazol-2-yl)-3,6-dihydropyrimidin-4-yl)cubane-1-carboxylic Acid (a Single Stereoisomer)

Converted from compounds VIIb-3.

By utilizing the analogous procedure of Method D, the title compound was synthesized as yellow solids. LC-MS (ESI): mass calcd. for C₂₄H₁₇ClFN₃O₄S 497.1, m/z found 498.1 [M+H]⁺. Chiral analysis (Column: Chiralpak IC 5 um 4.6*250 mm; Mobile Phase: Hex: EtOH:TFA=80:20:0.2 at 1 mL/min; Temp: 30° C.; Wavelength: 230 nm, R_{T}=9.145 min). ¹H NMR (400 MHz, CD₃OD) δ 7.90 (d, J=2.8 Hz, 1H), 7.74 (d, J=2.8 Hz, 1H), 7.41-7.38 (m, 1H), 7.23-7.20 (m, 1H), 7.06-7.02 (m, 1H), 6.12 (s, 1H), 4.25 (s, 6H), 3.62 (s, 3H).

### Compound Ib-4: (1R,2R,3R,8S)-4-((S*)-6-(2-chloro-3,4-difluorophenyl)-5-(methoxycarbonyl)-2-(thiazol-2-yl)-3,6-dihydropyrimidin-4-yl)cubane-1-carboxylic Acid (a Single Stereoisomer)

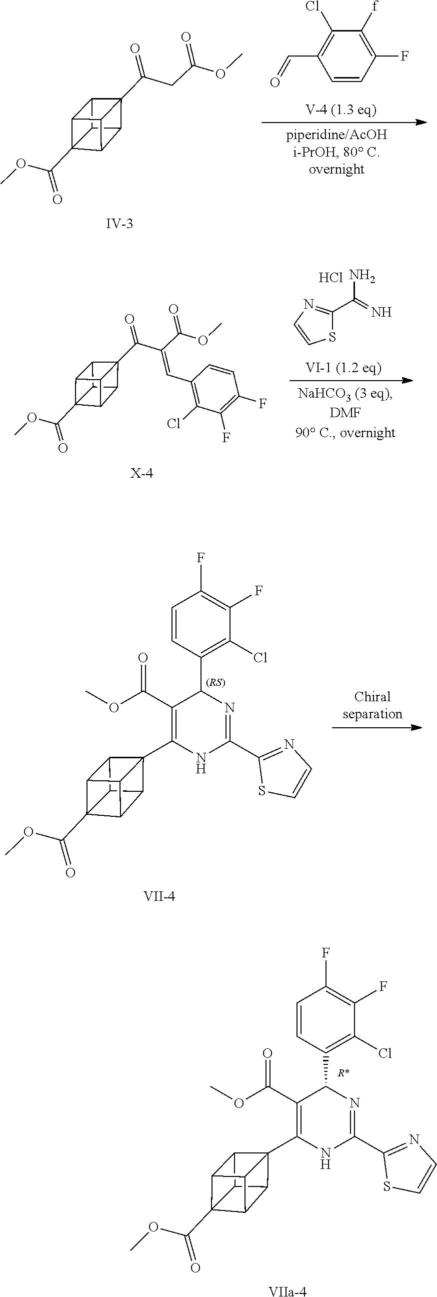

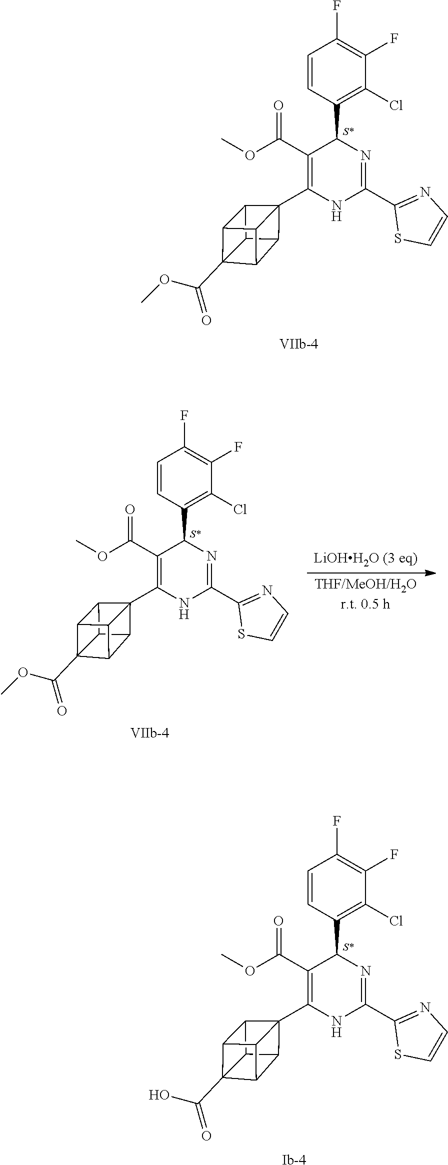 [SMILES: COC(=O)C1=C(C23C4C5C2C2C3C4C52C(=O)OC)NC(c2nccs2)=N[C@@H]1c1ccc(F)c(F)c1Cl ; "VIIb-4" ; COC(=O)C1=C(C23C4C5C2C2C3C4C52C(=O)OC)NC(c2nccs2)=N[C@@H]1c1ccc(F)c(F)c1Cl ; "VIIb-4" ; COC(=O)C1=C(C23C4C5C2C2C3C4C52C(=O)O)NC(c2nccs2)=N[C@@H]1c1ccc(F)c(F)c1Cl ; "Ib-4"]

### Intermediate X-4: (1r,2R,3r,8S)-methyl 4-(3-(2-chloro-3,4-difluorophenyl)-2-(methoxycarbonyl)acryloyl)cubane-1-carboxylate

Converted from compounds IV-3 and V-4.

By utilizing the analogous procedure of Method B step 1, the title compound was synthesized as yellow oil. ¹H NMR (300 MHz, CDCl₃) δ 7.90 (s, 0.4H), 7.67 (s, 0.6H), 7.23-7.20 (m, 0.6H), 7.15-7.08 (m, 1.4H), 4.43-4.41 (m, 1.4H), 4.40-4.38 (m, 0.6H), 4.31-4.29 (m, 1.4H), 4.25-4.22 (m, 0.6H), 4.10-4.07 (m, 1H), 4.03-4.01 (m, 1H), 3.87 (s, 1H), 3.76-3.74 (m, 2H), 3.73-3.72 (m, 2H), 3.69-3.68 (m, 1H).

### Intermediate VII-4: Methyl 4-(2-chloro-3,4-difluorophenyl)-6-((2r,3R,4r,5S)-4-(methoxycarbonyl)cuban-1-yl)-2-(thiazol-2-yl)-1,4-dihydropyrimidine-5-carboxylate (a Mixture of 2 Stereoisomers)

Converted from compounds X-4 and VI-1.

By utilizing the analogous procedure of Method B step 2, the title compound was synthesized as yellow solid. ¹H NMR (400 MHz, CDCl₃) δ 8.04 (s, 0.6H), 7.84 (d, J=3.2 Hz, 0.3H), 7.82 (d, J=3.2 Hz, 0.7H), 7.52 (d, J=3.2 Hz, 0.3H), 7.49 (s, 0.4H), 7.45 (d, J=3.2 Hz, 0.7H), 7.10-6.99 (m, 2H), 6.19 (s, 0.7H), 6.08 (d, J=2.8 Hz, 0.3H), 4.34-4.24 (m, 6H), 3.75 (s, 3H), 3.67 (s, 1H), 3.62 (s, 2H).

A racemic of methyl 4-(2-chloro-3,4-difluorophenyl)-6-((2r,3R,4r,5S)-4-(methoxycarbonyl)cuban-1-yl)-2-(thiazol-2-yl)-1,4-dihydropyrimidine-5-carboxylate VII-4 (520 mg, 0.883 mmol, 90% purity) was separated by chiral Prep. SFC (separation condition: Column: Chiralpak IE 5 um 20*250 mm; Mobile Phase: CO₂:IPA:DEA=70:30:0.3 at 50 g/min; Temp: 40° C.; Wavelength: 254 nm) to give (4R*)-methyl 4-(2-chloro-3,4-difluorophenyl)-6-((2R,3R,4R,5S)-4-(methoxycarbonyl)cuban-1-yl)-2-(thiazol-2-yl)-1,4-dihydropyrimidine-5-carboxylate VIIa-4 (240 mg, 90% purity from ¹H NMR, 46% yield, 100% ee) and (4S*)-methyl 4-(2-chloro-3,4-difluorophenyl)-6-((2R,3R,4R,5S)-4-(methoxycarbonyl)cuban-1-yl)-2-(thiazol-2-yl)-1,4-dihydropyrimidine-5-carboxylate VIIb-4 (240 mg, 90% purity from ¹H NMR, 46% yield, 97.1% ee) as yellow solids.

Intermediate VIIa-4 (a single stereoisomer): Chiral analysis (Column: Chiralpak IE 5 um 4.6*250 mm; Mobile Phase: CO₂ IPA:DEA=70:30:0.2 at 3 g/min; Temp: 40° C.; Wavelength: 254 nm, R_{T}=5.93 min). 1H NMR (400 MHz, CDCl₃) δ 8.04 (s, 0.6H), 7.83 (s, 1H), 7.51-7.45 (m, 1.4H), 7.10-7.00 (m, 2H), 6.19 (s, 0.7H), 6.08 (s, 0.3H), 4.31-4.25 (m, 6H), 3.74 (s, 3H), 3.66 (s, 1H), 3.62 (s, 2H).

Intermediate VIIb-4 (a single stereoisomer): Chiral analysis (Column: Chiralpak IE 5 um 4.6*250 mm; Mobile Phase: CO₂ IPA:DEA=70:30:0.2 at 3 g/min; Temp: 40° C.; Wavelength: 254 nm, R_{T}=6.93 min). ¹H NMR (400 MHz, CDCl₃) δ 8.04 (s, 0.6H), 7.83 (s, 1H), 7.52-7.45 (m, 1.4H), 7.10-6.99 (m, 2H), 6.19 (s, 0.7H), 6.08 (s, 0.3H), 4.33-4.25 (m, 6H), 3.74 (s, 3H), 3.66 (s, 1H), 3.62 (s, 2H).

### Compound Ib-4: (1R,2R,3R,8S)-4-((S*)-6-(2-chloro-3,4-difluorophenyl)-5-(methoxycarbonyl)-2-(thiazol-2-yl)-3,6-dihydropyrimidin-4-yl)cubane-1-carboxylic Acid (a Single Stereoisomer)

Converted from compounds VIIb-4.

By utilizing the analogous procedure of Method D, the title compound was synthesized as yellow solid. LC-MS (ESI): mass calcd. for C₂₄H₁₆ClF₂N₃O₄S 515.1, m/z found 516.1 [M+H]⁺. ¹H NMR (400 MHz, CD₃OD) δ 7.93 (d, J=3.2 Hz, 1H), 7.77 (d, J=2.8 Hz, 1H), 7.25-7.23 (m, 2H), 6.15 (s, 1H), 4.27 (br s, 6H), 3.65 (s, 3H).

### Compound Ib-5: (1R,2R,3R,8S)-4-((S*)-5-(ethoxycarbonyl)-6-(3-fluoro-2-methylphenyl)-2-(thiazol-2-yl)-3,6-dihydropyrimidin-4-yl)cubane-1-carboxylic Acid (a Single Stereoisomer)

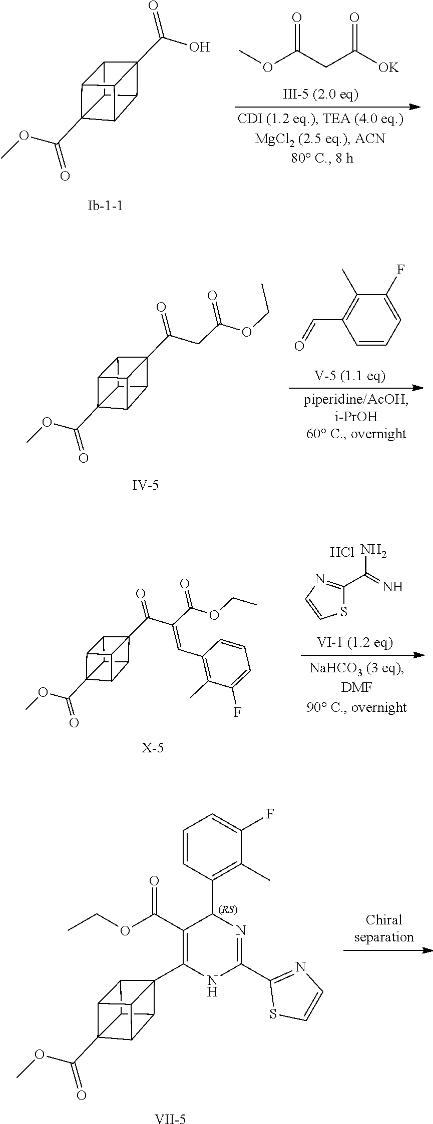

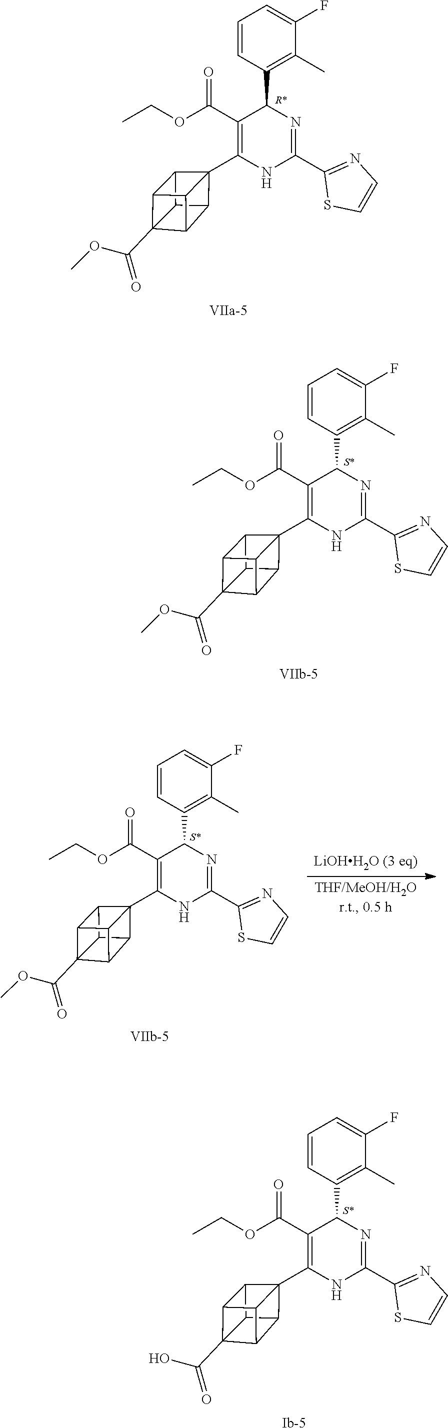

### Intermediate IV-5: (1r,2R,3r,8S)-methyl 4-(3-ethoxy-3-oxopropanoyl)cubane-1-carboxylate

Converted from compounds Ib-1-1 and III-5.

By utilizing the analogous procedure of Method A, the title compound was synthesized as yellow solid. ¹H NMR (400 MHz, CDCl₃) δ 4.32-4.14 (m, 8H), 3.72 (s, 3H), 3.48 (s, 1.3H), 3.44 (s, 0.7H), 1.29 (t, J=7.2 Hz, 3H).

### Intermediate X-5: (1r,2R,3r,8S)-methyl 4-(2-(ethoxycarbonyl)-3-(3-fluoro-2-methylphenyl)acryloyl)cubane-1-carboxylate

Converted from compounds IV-5 and V-5.

By utilizing the analogous procedure of Method B step 1, the title compound was synthesized as yellow oil. ¹H NMR (400 MHz, CDCl₃) δ 7.92 (s, 0.4H), 7.72 (s, 0.6H), 7.18-7.02 (m, 3H), 4.41-4.39 (m, 2H), 4.30-4.21 (m, 4H), 4.03-3.93 (m, 2H), 3.73 (m, 1.8H), 3.67 (s, 1.2H), 2.29 (d, J=2.0 Hz, 1.2H), 2.26 (d, J=2.0 Hz, 1.8H), 1.33 (t, J=7.2 Hz, 1.3H), 1.13 (t, J=7.2 Hz, 1.7H).

### Intermediate VII-5: Ethyl 4-(3-fluoro-2-methylphenyl)-6-((2r,3R,4r,5S)-4-(methoxycarbonyl)cuban-1-yl)-2-(thiazol-2-yl)-1,4-dihydropyrimidine-5-carboxylate (a Mixture of 2 Stereoisomers)

Converted from compounds X-5 and VI-1.

By utilizing the analogous procedure of Method B step 2, the title compound was synthesized as yellow solid. ¹H NMR (400 MHz, DMSO-d₆) δ 7.97 (s, 1H), 7.81-7.91 (m, 1H), 7.51 (d, J=3.2 Hz, 0.2H), 7.42 (d, J=2.8 Hz, 0.8H), 7.13-7.02 (m, 2H), 6.93-6.88 (m, 1H), 6.02 (s, 0.8H), 5.93 (d, J=1.6 Hz, 0.2H), 4.34-4.26 (m, 6H), 4.14-4.02 (m, 2H), 3.74 (s, 2.5H), 3.73 (s, 0.5H), 2.55 (d, J=1.6 Hz, 2.5H), 2.42 (d, J=1.6 Hz, 0.5H), 1.13-1.09 (m, 3H).

A racemic mixture of ethyl 4-(3-fluoro-2-methylphenyl)-6-((2r,3R,4r,5S)-4-(methoxycarbonyl)cuban-1-yl)-2-(thiazol-2-yl)-1,4-dihydropyrimidine-5-carboxylate VII-5 (310 mg, 90% purity, 0.552 mmol) was separated by chiral Prep. HPLC (separation condition: Column: Chiralpak IC 5 um 20*250 nm; Mobile Phase: Hex: IPA:DEA=80:20:0.2 at 15 mL/min; Temp: 30° C.; Wavelength: 254 nm) to give (4R*)-ethyl 4-(3-fluoro-2-methylphenyl)-6-((2R,3R,4R,5S)-4-(methoxycarbonyl)cuban-1-yl)-2-(thiazol-2-yl)-1,4-dihydropyrimidine-5-carboxylate VIIa-5 (142 mg, 46% yield, 90% purity from 1H NMR, 100% ee) as yellow solids and (4S*)-ethyl 4-(3-fluoro-2-methylphenyl)-6-((2R,3R,4R,5S)-4-(methoxycarbonyl)cuban-1-yl)-2-(thiazol-2-yl)-1,4-dihydropyrimidine-5-carboxylate VIIb-5 (160 mg, 52% yield, 90% purity from 1H NMR, 98.8% ee) as yellow solids.

Intermediate VIIa-5 (a single stereoisomer): Chiral analysis (Column: Chiralpak IC 5 μm 4.6*250 nm; Mobile Phase: Hex: IPA:DEA=80:20:0.2 at 1 mL/min; Temp: 30° C.; Wavelength: 254 nm, RT=8.083 min). 1H NMR (400 MHz, CDCl3) δ 7.80 (d, J=3.2 Hz, 1H), 7.44 (d, J=3.2 Hz, 1H), 7.11-7.04 (m, 2H), 6.93-6.89 (m, 1H), 6.01 (s, 1H), 4.32-4.27 (m, 6H), 4.15-4.00 (m, 2H), 3.74 (s, 3H), 2.53 (s, 3H), 1.11 (t, J=7.2 Hz, 3H).

Intermediate VIIb-5 (a single stereoisomer): Chiral analysis (Column: Chiralpak IC 5 μm 4.6*250 nm; Mobile Phase: Hex: IPA:DEA=80:20:0.2 at 1 mL/min; Temp: 30° C.; Wavelength: 254 nm, R_{T}=9.852 min). ¹H NMR (400 MHz, CDCl₃) δ 7.80 (d, J=3.2 Hz, 1H), 7.43 (d, J=2.8 Hz, 1H), 7.11-7.04 (m, 2H), 6.93-6.88 (m, 1H), 6.01 (s, 1H), 4.32-4.27 (m, 6H), 4.14-3.99 (m, 2H), 3.74 (s, 3H), 2.53 (s, 3H), 1.11 (t, J=7.2 Hz, 3H).

### Compound Ib-5: (1R,2R,3R,8S)-4-((S*)-5-(ethoxycarbonyl)-6-(3-fluoro-2-methylphenyl)-2-(thiazol-2-yl)-3,6-dihydropyrimidin-4-yl)cubane-1-carboxylic Acid (a Single Stereoisomer)

Converted from compounds VIIb-5.

By utilizing the analogous procedure of Method D, the title compound was synthesized as yellow solids. LC-MS (ESI): mass calcd. for C₂₆H₂₂FN₃O₄S 491.1, m/z found 492.2 [M+H]⁺. ¹H NMR (400 MHz, DMSO-d₆) δ 9.58 (s, 0.7H), 8.48 (s, 0.3H), 8.02-7.98 (m, 1.7H), 7.92 (d, J=3.2 Hz, 0.3H), 7.25-7.13 (m, 1.7H), 7.06-7.02 (m, 1.3H), 5.87 (s, 0.3H), 5.74 (s, 0.7H), 4.23-4.08 (m, 6H), 4.06-3.96 (m, 2H), 2.46 (s, 1H), 2.41 (s, 2H), 1.04 (t, J=6.8 Hz, 3H).

### Compound Ib-6: (2S,3S,5S,6S,7S,8S)-4-((S*)-6-(2-chloro-4-fluorophenyl)-5-(ethoxycarbonyl)-2-(thiazol-2-yl)-3,6-dihydropyrimidin-4-yl)cubane-1-carboxylic Acid

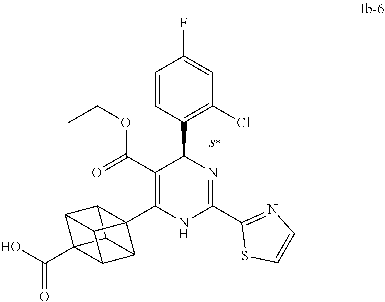 [SMILES: CCOC(=O)C1=C(C23C4C5C2C2C3C4C52C(=O)O)NC(c2nccs2)=N[C@@H]1c1ccc(F)cc1Cl]

The synthesis of compound Ib-6 is similar with compound Ib-4.

LC-MS (ESI): mass calcd. for C₂₅H₁₆ClFN₃O₄S 511.9, m/z found 512.0 [M+H]⁺. ¹H NMR (400 MHz, DMSO-d₆) δ 8.49 (s, 1H), 8.01-7.97 (m, 1.5H), 7.94-7.92 (m, 0.5H), 7.43-7.37 (m, 2H), 7.23-7.18 (m, 1H), 6.03 (s, 0.4H), 5.94 (s, 0.6H), 4.17-4.11 (m, 6H), 4.04-3.98 (m, 2H), 1.08-1.02 (m, 3H).

### Compound Ib-7: (2S,3S,5S,6S,7S,8S)-4-((S*)-6-(2-chloro-3,4-difluorophenyl)-5-(ethoxycarbonyl)-2-(thiazol-2-yl)-3,6-dihydropyrimidin-4-yl)cubane-1-carboxylic Acid

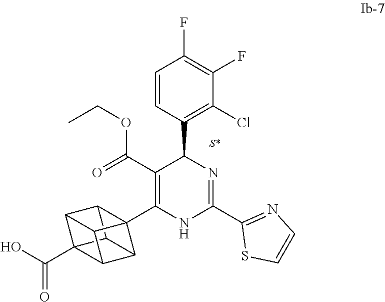 [SMILES: CCOC(=O)C1=C(C23C4C5C2C2C3C4C52C(=O)O)NC(c2nccs2)=N[C@@H]1c1ccc(F)c(F)c1Cl]

The synthesis of compound Ib-7 is similar with compound Ib-4.

LC-MS (ESI): mass calcd. for C₂₅H₁₈ClF₂N₃O₄S 529.1, m/z found 530.2 [M+H]⁺. ¹H NMR (400 MHz, CD₃OD) δ 7.94 (d, J=3.2 Hz, 1H), 7.77 (d, J=3.2 Hz, 1H), 7.29-7.21 (m, 2H), 6.17 (s, 1H), 4.27 (s, 6H), 4.14-4.03 (m, 2H), 1.13 (t, J=7.2 Hz, 3H).

### Compound Ia-1: (2R,3R,5R,6R,7R,8R)-4-((R*)-6-(3-fluoro-2-methylphenyl)-5-(methoxycarbonyl)-2-(thiazol-2-yl)-3,6-dihydropyrimidin-4-yl)cubane-1-carboxylic Acid

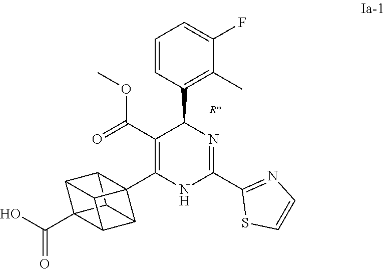 [SMILES: COC(=O)C1=C(C23C4C5C2C2C3C4C52C(=O)O)NC(c2nccs2)=N[C@@H]1c1cccc(F)c1C]

The synthesis of compound Ia-1 is similar with compound Ib-4.

LC-MS (ESI): mass calcd. for C₂₅H₂₀FN₃O₄S 477.1, m/z found 478.1 [M+H]⁺. ¹H NMR (400 MHz, DMSO-d₆) δ 7.99-7.91 (m, 2H), 7.20-7.13 (m, 1.7H), 7.05-7.00 (m, 1.3H), 5.85 (s, 0.3H), 5.74 (s, 0.7H), 4.20-4.11 (m, 6H), 3.58 (s, 1H), 3.56 (s, 2H), 2.46 (s, 1H), 2.40 (s, 2H).

### Compound Ib-8: (1S,2R,3S,8S)-4-((S*)-6-(2-chloro-3-fluorophenyl)-5-(methoxycarbonyl)-2-(thiazol-2-yl)-3,6-dihydropyrimidin-4-yl)cubane-1-carboxylic Acid

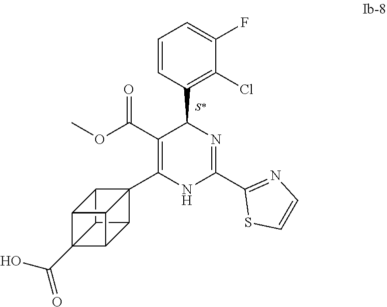 [SMILES: COC(=O)C1=C(C23C4C5C2C2C3C4C52C(=O)O)NC(c2nccs2)=N[C@@H]1c1cccc(F)c1Cl]

The synthesis of compound Ib-8 is similar with compound Ib-4.

LC-MS (ESI): mass calcd. for C₂₄H₁₇ClFN₃O₄S 497.1, m/z found 498.1 [M+H]⁺. ¹H NMR (400 MHz, CD₃OD) δ 7.94 (d, J=3.2 Hz, 1H), 7.77 (d, J=2.8 Hz, 1H), 7.24-7.37 (m, 2H), 7.15-9.19 (m, 1H), 6.21 (s, 1H), 4.29 (s, 6H), 3.65 (s, 3H).

### Compound Ia-2: (R*)-4-(6-(2-chloro-3,4-difluorophenyl)-5-(methoxycarbonyl)-2-(1-methyl-1H-imidazol-2-yl)-3,6-dihydropyrimidin-4-yl)cubane-1-carboxylic Acid

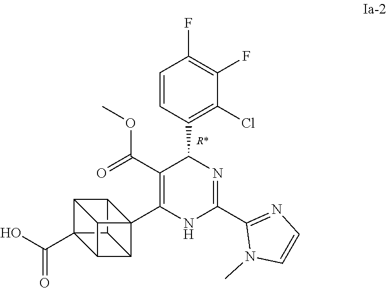 [SMILES: COC(=O)C1=C(C23C4C5C2C2C3C4C52C(=O)O)NC(c2nccn2C)=N[C@H]1c1ccc(F)c(F)c1Cl]

The synthesis of compound Ia-2 is similar with compound Ib-4.

LC-MS (ESI): mass calcd. for C₂₅H₁₆ClF₂N₄O₄ 512.1, m/z found 513.1 [M+H]⁺. ¹H NMR (400 MHz, DMSO-d₆) δ 8.53-8.42 (m, 1H), 7.47-7.35 (m, 2H), 7.18-7.06 (m, 1H), 7.02 (s, 1H), 6.02 (s, 0.7H), 5.85 (s, 0.3H), 4.19-4.10 (m, 6H), 4.04 (s, 1H), 3.85 (s, 2H), 3.58 (s, 1.5H), 2.57 (s, 1.5H).

### Compound Ib-11: (S*)-4-(6-(2-chloro-3,4-difluorophenyl)-5-(propoxycarbonyl)-2-(thiazol-2-yl)-3,6-dihydropyrimidin-4-yl)cubane-1-carboxylic Acid

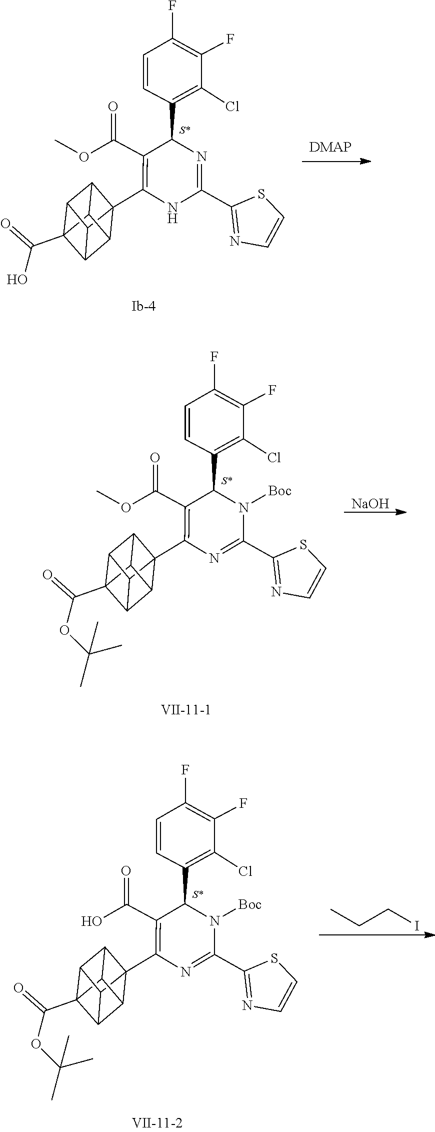

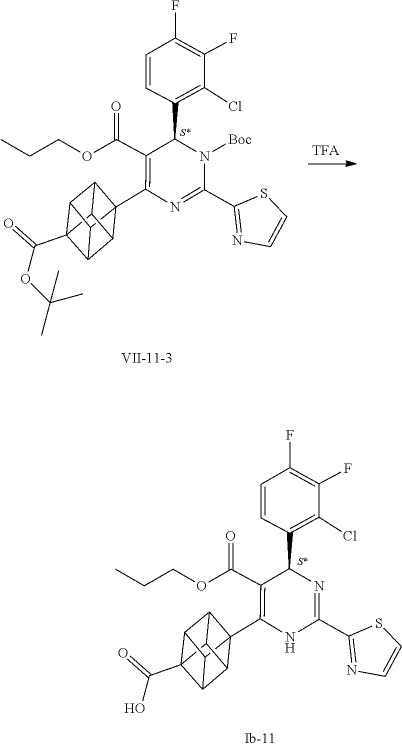 [SMILES: CCCOC(=O)C1=C(C23C4C5C2C2C3C4C52C(=O)OC(C)(C)C)N=C(c2nccs2)N(C(=O)OC(C)(C)C)[C@@H]1c1ccc(F)c(F)c1Cl ; CCCOC(=O)C1=C(C23C4C5C2C2C3C4C52C(=O)O)NC(c2nccs2)=N[C@@H]1c1ccc(F)c(F)c1Cl]

### Intermediate VII-11-1: (S*)-1-tert-butyl 5-methyl 4-(4-(tert-butoxycarbonyl)cuban-1-yl)-6-(2-chloro-3,4-difluorophenyl)-2-(thiazol-2-yl)pyrimidine-1,5(6H)-dicarboxylate

To a solution of di-tert-butyl dicarbonate (420 mg, 1.92 mmol) and N,N-dimethylpyridin-4-amine (200 mg, 1.64 mmol) in tetrahydrofuran (15 mL) was added compound Ib-4 (300 mg, 98% purity, 0.570 mmol). After stirring at 60° C. for 12 hours, the mixture was cooled to room temperature, poured into water (50 mL) and extracted with ethyl acetate (80 mL) twice. The combined organic layers were dried over Na₂SO₄(s), filtered and concentrated to afford a residue, which was purified by C18 column (acetonitrile:water=05% to 95%) to give the desired product (200 mg, 47% yield) as a yellow solid. ¹H NMR (400 MHz, CDCl₃) δ 7.89 (s, 1H), 7.50 (s, 1H), 6.95-6.89 (m, 2H), 6.70 (s, 1H), 4.23 (s, 6H), 3.73 (s, 3H), 1.49 (s, 9H), 1.26 (s, 9H).

### Intermediate VII-11-2: (S*)-1-(tert-butoxycarbonyl)-4-(4-(tert-butoxycarbonyl)cuban-1-yl)-6-(2-chloro-3,4-difluorophenyl)-2-(thiazol-2-yl)-1,6-dihydropyrimidine-5-carboxylic Acid

To a solution of Intermediate VII-11-1 (200 mg, 90% purity, 0.268 mmol) in tetrahydrofuran (3 mL) and ethanol (3 ml) was added a solution of sodium hydroxide (80 mg, 2 mmol) in water (1 mL). After stirring at room temperature for 2 hours, the mixture was acidified to pH=5 with 1 M hydrochloride aqueous solution. The obtained mixture was extracted with ethyl acetate (10 mL) for three times. The combined organic layers were washed with brine (20 mL), dried over Na₂SO₄(s) and filtered. The filtrate was concentrated under reduced pressure to give a residue, which was purified by C18 (acetonitrile:water=20% to 85%) to give the desired compound (70 mg, 36% yield) as yellow solids. ¹H NMR (400 MHz, CDCl₃) δ 7.87 (d, J=2.8 Hz, 1H), 7.48 (d, J=3.2 Hz, 1H), 6.91-6.88 (m, 2H), 6.68 (s, 1H), 4.23 (s, 6H), 1.49 (s, 9H), 1.26 (s, 9H).

### Intermediate VII-11-3: (S*)-1-tert-butyl 5-propyl 4-(4-(tert-butoxycarbonyl)cuban-1-yl)-6-(2-chloro-3,4-difluorophenyl)-2-(thiazol-2-yl)pyrimidine-1,5(6H)-dicarboxylate

To a solution of Intermediate VII-11-2 (70 mg, 0.096 mmol) in N,N-dimethylformamide (2 mL) was added 1-iodopropane (90 mg, 0.436 mmol) and potassium carbonate (60 mg, 0.434 mmol) at room temperature. After stirring at 30° C. for two hours, the mixture was concentrated in vacuo to give a residue, which was purified by C18 column (acetonitrile water=50% to 95%) to give the desired product (60 mg, 64% yield) as a yellow solid. LC-MS (ESI): mass calcd. for C₃₅H₃₆ClF₂N₃O₆S 699.2, m/z found 700.4 [M+H]⁺.

### Compound Ib-11: (S*)-4-(6-(2-chloro-3,4-difluorophenyl)-5-(propoxycarbonyl)-2-(thiazol-2-yl)-3,6-dihydropyrimidin-4-yl)cubane-1-carboxylic Acid

To a solution of Intermediate VII-11-3 (50 mg, 71% purity, 0.051 mmol) in dichloromethane (2 mL) was added trifluoroacetic acid (2 mL) at 0° C. After stirring at room temperature overnight, the reaction mixture was concentrated under reduced pressure to give the crude product, which was purified by C18 column (acetonitrile:water=20% to 70%) to give the title compound (25 mg, 90% yield) as a yellow solid. LC-MS (ESI): mass calcd. for C₂₆H₂₀ClF₂N₃O₄S 543.1, m/z found 543.8 [M+H]⁺. ¹H NMR (400 MHz, CD₃OD) δ 7.94 (d, J=3.2 Hz, 1H), 7.78 (s, 1H), 7.27-7.24 (m, 2H), 6.19 (s, 1H), 4.28 (s, 6H), 4.02-3.99 (m, 2H), 1.59-1.50 (m, 2H), 0.77 (d, J=7.2 Hz, 3H).

### Compound Ib-12: (S*)-6-(6-Carboxy-cuban-3-yl)-4-(2-chloro-3,4-difluoro-phenyl)-2-thiazol-2-yl-1,4-dihydro-pyrimidine-5-carboxylic Acid Isopropyl Ester

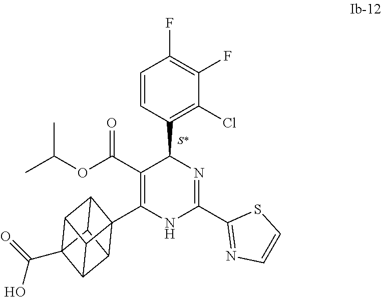 [SMILES: CC(C)OC(=O)C1=C(C23C4C5C2C2C3C4C52C(=O)O)NC(c2nccs2)=N[C@@H]1c1ccc(F)c(F)c1Cl]

The synthesis of compound Ib-12 is similar with compound Ib-11.

LC-MS (ESI): mass calcd. for C₂₆H₂₀ClF₂N₃O₄S 543.1, m/z found 544.1 [M+H]⁺. ¹H NMR (400 MHz, CD₃OD) δ 7.91 (d, J=3.2 Hz, 1H), 7.75-7.74 (m, 1H), 7.26-7.22 (m, 2H), 6.14 (s, 1H), 4.91-4.88 (m, 1H), 4.24 (s, 6H), 1.21 (d, J=6.0 Hz, 3H), 0.90 (d, J=6.4 Hz, 3H).

### Compound Ia-3: (1R,2R,3R,8S)-4-((R)-6-(3,4-difluoro-2-methylphenyl)-5-(methoxycarbonyl)-2-(thiazol-2-yl)-3,6-dihydropyrimidin-4-yl)cubane-1-carboxylic Acid

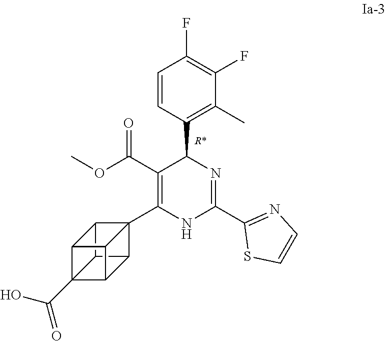 [SMILES: COC(=O)C1=C(C23C4C5C2C2C3C4C52C(=O)O)NC(c2nccs2)=N[C@@H]1c1ccc(F)c(F)c1C]

The synthesis of compound Ia-3 is similar with compound Ib-4.

LC-MS (ESI): mass calcd. for C₂₅H₁₉F₂N₃O₄S 495.1, m/z found 496.1 [M+H]⁺. ¹H NMR (400 MHz, CD₃OD) δ 7.92 (d, J=3.2 Hz, 1H), 7.75 (d, J=3.2 Hz, 1H), 7.06-7.02 (m, 2H), 5.91 (s, 1H), 4.28 (s, 6H), 3.66 (s, 3H), 2.56 (s, 3H).

### Compound Ib-13: (2S,3S,5S,6S,7S,8S)-4-((S*)-6-(2-bromo-3,4-difluorophenyl)-5-(methoxycarbonyl)-2-(thiazol-2-yl)-3,6-dihydropyrimidin-4-yl)cubane-1-carboxylic Acid

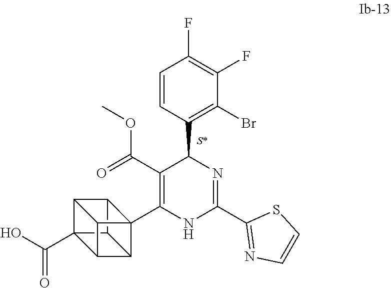 [SMILES: COC(=O)C1=C(C23C4C5C2C2C3C4C52C(=O)O)NC(c2nccs2)=N[C@@H]1c1ccc(F)c(F)c1Br]

The synthesis of compound Ib-13 is similar with compound Ib-4.

LC-MS (ESI): mass calcd. for C₂₄H₁₆BrF₂N₃O₄S 559.0, m/z found 560.0 [M+H]⁺. ¹H NMR (400 MHz, DMSO-d₆) δ 8.52 (br s, 1H), 8.01-7.93 (m, 2H), 7.51-7.44 (m, 1H), 7.23-7.17 (m, 1H), 6.00 (s, 0.5H), 5.93 (s, 0.5H), 4.20-4.13 (m, 6H), 3.56 (s, 3H).

### Compound Ib-14: (2S,3S,5S,6S,7S,8S)-4-((S*)-6-(2-bromo-3,4-difluorophenyl)-5-(ethoxycarbonyl)-2-(thiazol-2-yl)-3,6-dihydropyrimidin-4-yl)cubane-1-carboxylic Acid

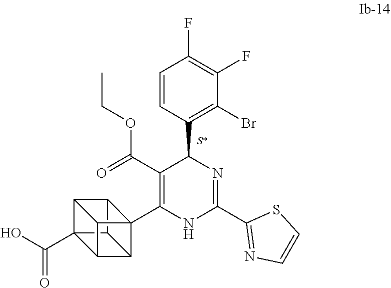 [SMILES: CCOC(=O)C1=C(C23C4C5C2C2C3C4C52C(=O)O)NC(c2nccs2)=N[C@@H]1c1ccc(F)c(F)c1Br]

The synthesis of compound Ib-14 is similar with compound Ib-4.

LC-MS (ESI): mass calcd. for C₂₅H₁₈BrF₂N₃O₄S 573.0, m/z found 574.0 [M+H]⁺. ¹H NMR (400 MHz, DMSO-d₆) δ 8.49 (br s, 1H), 8.01-7.93 (m, 2H), 7.53-7.44 (m, 1H), 7.25-7.20 (m, 1H), 6.03 (s, 0.5H), 5.94 (s, 0.5H), 4.18-4.11 (m, 6H), 4.04-3.98 (m, 2H), 1.07-1.01 (m, 3H).

### Compound Ib-15: (2S,3S,5S,6S,7S,8S)-4-((S*)-6-(2-chloro-3-fluorophenyl)-5-(ethoxycarbonyl)-2-(thiazol-2-yl)-3,6-dihydropyrimidin-4-yl)cubane-1-carboxylic Acid

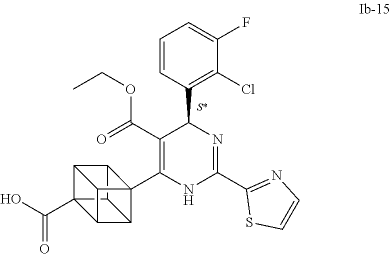 [SMILES: CCOC(=O)C1=C(C23C4C5C2C2C3C4C52C(=O)O)NC(c2nccs2)=N[C@@H]1c1cccc(F)c1Cl]

The synthesis of compound Ib-15 is similar with compound Ib-4.

LC-MS (ESI): mass calcd. for C₂₅H₁₉ClFN₃O₄S 511.1, m/z found 512.1 [M+H]⁺. ¹H NMR (400 MHz, CD₃OD) δ 7.93 (d, J=3.2 Hz, 1H), 7.76 (d, J=2.8 Hz, 1H), 7.33-7.25 (m, 2H), 7.18-7.14 (m, 1H), 6.23 (s, 1H), 4.27 (s, 6H), 4.12-4.04 (m, 2H), 1.12 (t, J=7.2 Hz 3H).

### Compound Ib-16: (2S,3S,5S,6S,7S,8S)-4-((S*)-6-(2-bromo-4-fluorophenyl)-5-(methoxycarbonyl)-2-(thiazol-2-yl)-3,6-dihydropyrimidin-4-yl)cubane-1-carboxylic Acid

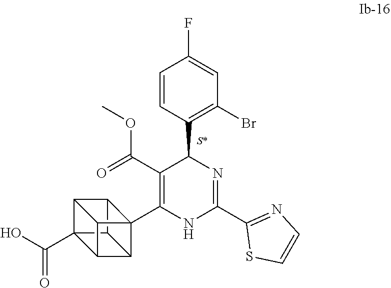 [SMILES: COC(=O)C1=C(C23C4C5C2C2C3C4C52C(=O)O)NC(c2nccs2)=N[C@@H]1c1ccc(F)cc1Br]

The synthesis of compound Ib-16 is similar with compound Ib-4.

LC-MS (ESI): mass calcd. for C₂₄H₁₇BrFN₃O₄S 541.0, m/z found 542.0 [M+H]⁺. ¹H NMR (400 MHz, CD₃OD) δ 7.94 (d, J=3.2 Hz, 1H), 7.77 (s, 1H), 7.44-7.42 (m, 2H), 7.14-7.10 (m, 1H), 6.14 (s, 1H), 4.28 (s, 6H), 3.65 (s, 3H).

### Compound Ib-17: (2S,3S,5S,6S,7S,8S)-4-((S*)-6-(2-bromo-3-fluorophenyl)-5-(methoxycarbonyl)-2-(thiazol-2-yl)-3,6-dihydropyrimidin-4-yl)cubane-1-carboxylic Acid

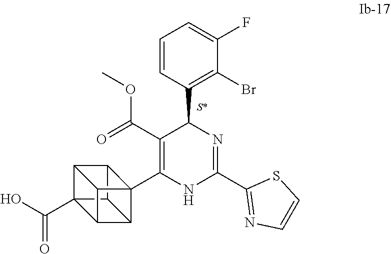 [SMILES: COC(=O)C1=C(C23C4C5C2C2C3C4C52C(=O)O)NC(c2nccs2)=N[C@@H]1c1cccc(F)c1Br]

The synthesis of compound Ib-17 is similar with compound Ib-4.

LC-MS (ESI): mass calcd. for C₂₄H₁₇BrFN₃O₄S 541.0, m/z found 542.0 [M+H]⁺. ¹H NMR (400 MHz, CD₃OD) δ 7.93 (d, J=3.2 Hz, 1H), 7.75 (d, J=2.4 Hz, 1H), 7.36-7.31 (m, 1H), 7.24-7.22 (m, 1H), 7.14-7.10 (m, 1H), 6.20 (s, 1H), 4.26 (s, 6H), 3.64 (s, 3H).

### Compound Ib-18: (S*)-4-(2-(3,5-difluoropyridin-2-yl)-5-(ethoxycarbonyl)-6-(3-fluoro-2-methylphenyl)-3,6-dihydropyrimidin-4-yl)cubane-1-carboxylic Acid

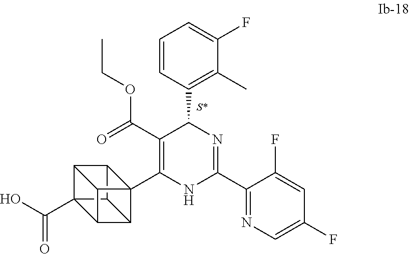 [SMILES: CCOC(=O)C1=C(C23C4C5C2C2C3C4C52C(=O)O)NC(c2ncc(F)cc2F)=N[C@H]1c1cccc(F)c1C]

The synthesis of compound Ib-18 is similar with compound Ib-4.

LC-MS (ESI): mass calcd. for C₂₈H₂₂F₃N₃O₄ 521.2, m/z found 522.2 [M+H]⁺. ¹H NMR (400 MHz, CD₃OD) δ 8.46 (d, J=2.0 Hz, 1H), 7.75-7.69 (m, 1H), 7.18-7.12 (m, 2H), 6.98-6.93 (m, 1H), 6.05 (s, 1H), 4.29 (s, 6H), 4.18-4.08 (m, 2H), 2.54 (s, 3H), 1.16 (t, J=7.2 Hz, 3H).

### Compound Ia-4: (R*)-4-(6-(2-chloro-3,4-difluorophenyl)-5-(ethoxycarbonyl)-2-(1-methyl-1H-imidazol-2-yl)-3,6-dihydropyrimidin-4-yl)cubane-1-carboxylic Acid

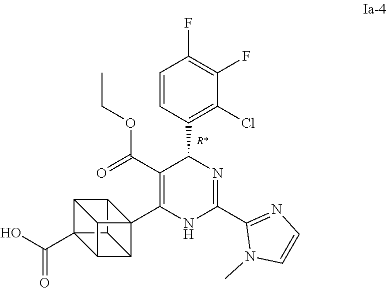 [SMILES: CCOC(=O)C1=C(C23C4C5C2C2C3C4C52C(=O)O)NC(c2nccn2C)=N[C@H]1c1ccc(F)c(F)c1Cl]

The synthesis of compound Ia-4 is similar with compound Ib-4.

LC-MS (ESI): mass calcd. for C₂₆H₂₁ClF₂N₄O₄ 526.1, m/z found 527.1 [M+H]⁺. ¹H NMR (400 MHz, CD₃OD) δ 7.27-7.17 (m, 3H), 7.03 (s, 1H), 6.18 (s, 1H), 4.28 (m, 6H), 4.14-4.06 (m, 2.4H), 3.90 (br s, 2.6H), 1.12 (t, J=7.2 Hz, 3H).

### Compound Ib-19: (1S,2R,3S,8S)-4-((S*)-6-(2-bromo-3-fluorophenyl)-5-(ethoxycarbonyl)-2-(thiazol-2-yl)-3,6-dihydropyrimidin-4-yl)cubane-1-carboxylic Acid

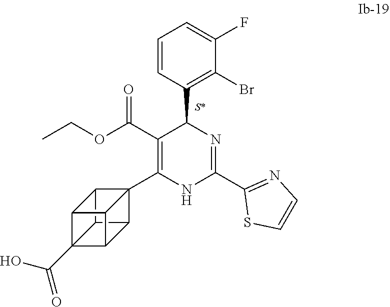 [SMILES: CCOC(=O)C1=C(C23C4C5C2C2C3C4C52C(=O)O)NC(c2nccs2)=N[C@@H]1c1cccc(F)c1Br]

The synthesis of compound Ib-19 is similar with compound Ib-4.

LC-MS (ESI): mass calcd. for C₂₅H₁₉BrFN₃O₄S 555.0, m/z found 556.1 [M+H]⁺. ¹H NMR (400 MHz, CD₃OD) δ 7.80 (d, J=3.2 Hz, 1H), 7.63 (s, 1H), 7.24-7.19 (m, 1H), 7.12 (d, J=7.2 Hz, 1H), 7.02-6.98 (m, 1H), 6.09 (s, 1H), 4.15 (s, 6H), 3.98-3.90 (m, 2H), 0.99 (t, J=7.2 Hz, 3H).

### Compound Ib-20: (S*)-4-(5-(ethoxycarbonyl)-2-(thiazol-2-yl)-6-(2,3,4-trifluorophenyl)-3,6-dihydropyrimidin-4-yl)cubane-1-carboxylic Acid

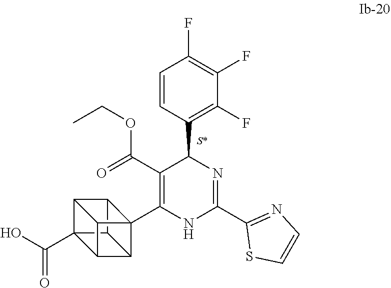 [SMILES: CCOC(=O)C1=C(C23C4C5C2C2C3C4C52C(=O)O)NC(c2nccs2)=N[C@@H]1c1ccc(F)c(F)c1F]

The synthesis of compound Ib-20 is similar with compound Ib-4.

LC-MS (ESI): mass calcd. For C₂₅H₁₈F₃N₃O₄S, 513.1 m/z found 514.2 [M+H]⁺. ¹H NMR (400 MHz, CD₃OD) δ 7.96 (d, J=2.8 Hz, 1H), 7.80 (d, J=2.8 Hz, 1H), 7.23-7.17 (m, 1H), 7.13-7.07 (m, 1H), 6.04 (s, 1H), 4.27 (s, 6H), 4.13 (q, J=7.2 Hz, 2H), 1.19 (t, J=7.2 Hz, 3H).

### Compound Ib-21: (S*)-4-(6-(4-fluoro-2-methylphenyl)-5-(methoxycarbonyl)-2-(thiazol-2-yl)-3,6-dihydropyrimidin-4-yl)cubane-1-carboxylic Acid

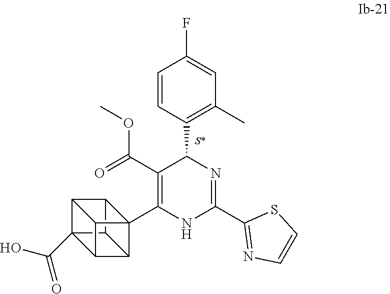 [SMILES: COC(=O)C1=C(C23C4C5C2C2C3C4C52C(=O)O)NC(c2nccs2)=N[C@H]1c1ccc(F)cc1C]

The synthesis of compound Ib-21 is similar with compound Ib-4.

LC-MS (ESI): mass calcd. For C₂₅H₂₀FN₃O₄S 477.1, m/z found 478.2 [M+H]⁺. ¹H NMR (400 MHz, CD₃OD) δ 7.90 (d, J=2.8 Hz, 1H), 7.73 (s, 1H), 7.27 (t, J=7.2 Hz, 1H), 6.91 (d, J=9.6 Hz, 1H), 6.84 (t, J=8.4 Hz, 1H), 5.89 (s, 1H), 4.26 (s, 6H), 3.63 (s, 3H), 2.60 (s, 3H).

### Compound Ib-22: (S*)-4-((2,3,4-trifluorophenyl)-6-(methoxycarbonyl)-2-(thiazolel-4-yl))-1,4-dihydropyrimidin-4-yl)cubane-5-carboxylic Acid

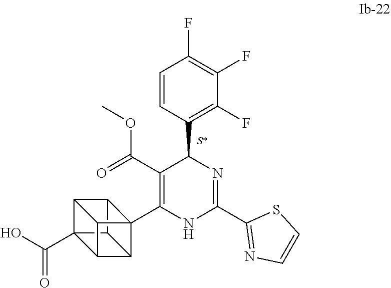 [SMILES: COC(=O)C1=C(C23C4C5C2C2C3C4C52C(=O)O)NC(c2nccs2)=N[C@@H]1c1ccc(F)c(F)c1F]

The synthesis of compound Ib-22 is similar with compound Ib-4.

LC-MS (ESI): mass calcd. for C₂₄H₁₆F₃N₃O₄S 499.4, m/z found 500.1 [M+H]⁺. ¹H NMR (400 MHz, CD₃OD) δ 7.95 (d, J=3.2 Hz, 1H), 7.79 (d, J=3.2 Hz, 1H), 7.20-7.14 (m, 1H), 7.11-7.04 (m, 1H), 6.01 (s, 1H), 4.25 (s, 6H), 3.68 (s, 3H).

### Compound Ib-23: (S*)-4-(6-(4-chloro-3-fluorophenyl)-5-(ethoxycarbonyl)-2-(thiazol-2-yl)-3,6-dihydropyrimidin-4-yl)cubane-1-carboxylic Acid

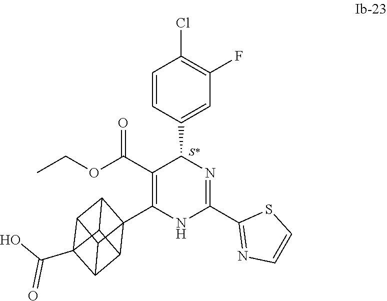 [SMILES: CCOC(=O)C1=C(C23C4C5C2C2C3C4C52C(=O)O)NC(c2nccs2)=N[C@H]1c1ccc(Cl)c(F)c1]

The synthesis of compound Ib-23 is similar with compound Ib-4.

LC-MS (ESI): mass calcd. for C₂₅H₁₆ClFN₃O₄S 511.1, m/z found 512.1 [M+H]⁺. ¹H NMR (400 MHz, DMSO-d₆) δ 8.04-8.01 (m, 1.7H), 7.97 (m, 0.3H), 7.60-7.53 (m, 1H), 7.29 (m, 0.3H), 7.23 (m, 0.7H), 7.18 (m, 0.3H), 7.14 (m, 0.7H), 5.68 (s, 0.3H), 5.51 (s, 0.7H), 4.14-4.10 (m, 8H), 1.15-1.10 (m, 3H).

### Compound Ib-24: (2S,3S,5S,6S,7S,8S)-4-((S*)-5-(ethoxycarbonyl)-6-(4-fluorophenyl)-2-(thiazol-2-yl)-3,6-dihydropyrimidin-4-yl)cubane-1-carboxylic Acid

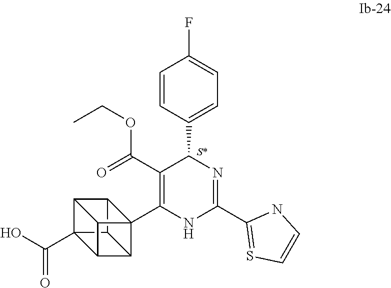

The synthesis of compound Ib-24 is similar with compound Ib-4.

LC-MS (ESI): mass calcd. For C₂₅H₂₀FN₃O₄S 477.1, m/z found 478.1 [M+H]⁺. ¹H NMR (400 MHz, CDCl₃) δ 8.06 (br s, 1H), 7.87 (d, J=2.4 Hz, 1H), 7.52-7.49 (m, 1H), 7.38-7.35 (m, 2H), 7.00-6.96 (t, J=8.8 Hz, 2H), 5.83 (s, 1H), 4.34-4.26 (m, 6H), 4.14 (q, J=7.2 Hz, 2H), 1.21-1.18 (t, J=6.8 Hz, 3H).

### Compound Ib-25: (2S,3S,5S,6S,7S,8S)-4-((S*)-6-(2-chlorophenyl)-5-(ethoxycarbonyl)-2-(thiazol-2-yl)-3,6-dihydropyrimidin-4-yl)cubane-1-carboxylic Acid

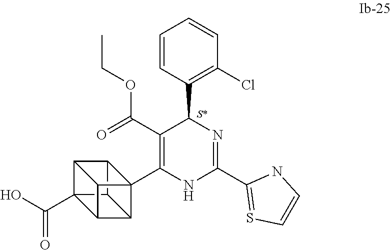

The synthesis of compound Ib-25 is similar with compound Ib-4.

LC-MS (ESI): mass calcd. for C₂₅H₂₀ClN₃O₄S, 493.1, m/z found 494.1 [M+H]⁺. ¹H NMR (400 MHz, CD₃OD) δ 7.80 (d, J=3.2 Hz, 1H), 7.63 (d, J=2.8 Hz, 1H), 7.33-7.29 (m, 2H), 7.18-7.12 (m, 2H), 6.08 (s, 1H), 4.14 (s, 6H), 4.00-3.90 (m, 2H), 1.03 (t, J=7.2 Hz, 3H).

### Compound Ia-5: (R*)-4-(5-(ethoxycarbonyl)-6-(4-fluoro-2-methylphenyl)-2-(thiazol-2-yl)-3,6-dihydropyrimidin-4-yl)cubane-1-carboxylic Acid

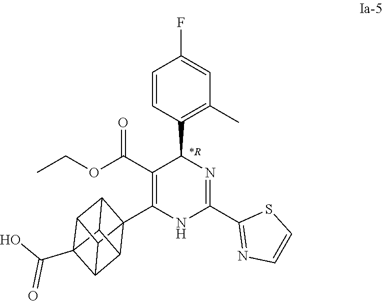 [SMILES: CCOC(=O)C1=C(C23C4C5C2C2C3C4C52C(=O)O)NC(c2nccs2)=N[C@@H]1c1ccc(F)cc1C]

The synthesis of compound Ia-5 is similar with compound Ib-4.

LC-MS (ESI): mass calcd. for C₂₆H₂₂FN₃O₄S 491.5, m/z found 492.1 [M+H]⁺. ¹H NMR (400 MHz, CD₃OD) δ 7.80 (d, J=3.2 Hz, 1H), 7.63 (d, J=3.2 Hz, 1H), 7.21-7.15 (m, 1H), 6.83-6.73 (m, 2H), 5.80 (s, 1H), 4.15 (s, 6H), 4.02-3.93 (m, 2H), 2.50 (s, 3H), 1.03 (t, J=7.2 Hz, 3H).

### Compound Ia-6: (R*)-4-(6-(2-bromo-4-fluorophenyl)-5-(ethoxycarbonyl)-2-(thiazol-2-yl)-3,6-dihydropyrimidin-4-yl)cubane-1-carboxylic Acid

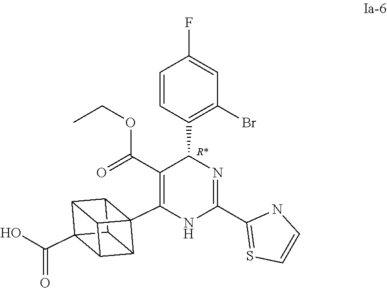

The synthesis of compound Ia-6 is similar with compound Ib-4.

LC-MS (ESI): mass calcd. for C₂₅H₁₉BrFN₃O₄S 556.4, m/z found 556.1 [M+H]⁺. ¹H NMR (400 MHz, DMSO-d₆) δ 8.00 (d, J=2.4 Hz, 1.5H), 7.93 (d, J=3.2 Hz, 0.5H), 7.58-7.54 (m, 1H), 7.39-7.35 (m, 1H), 7.29-7.22 (m, 1H), 6.00 (s, 0.5H), 5.91 (s, 0.5H), 4.17-4.10 (m, 6H), 4.05-3.98 (m, 2H), 1.08-1.02 (m, 3H).

### Compound Ib-26: (S*)-4-(2-(3,5-difluoropyridin-2-yl)-5-(methoxycarbonyl)-6-(3-fluoro-2-methylphenyl)-3,6-dihydropyrimidin-4-yl)cubane-1-carboxylic Acid

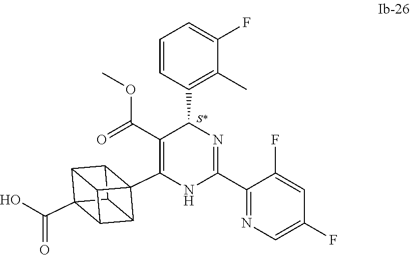 [SMILES: COC(=O)C1=C(C23C4C5C2C2C3C4C52C(=O)O)NC(c2ncc(F)cc2F)=N[C@H]1c1cccc(F)c1C]

The synthesis of compound Ib-26 is similar with compound Ib-4.

LC-MS (ESI): mass calcd. for C₂₇H₂₀F₃N₃O₄ 507.1, m/z found 508.1 [M+H]⁺. ¹H NMR (400 MHz, DMSO-d₆) δ 9.16 (s, 0.4H), 8.74 (s, 0.6H), 8.57 (d, J=2.4 Hz, 0.6H), 8.54 (d, J=2.4 Hz, 0.4H), 8.09-8.01 (m, 1H), 7.26-7.15 (m, 1.3H), 7.08-6.96 (m, 1.7H), 5.90 (s, 0.6H), 5.76 (s, 0.4H), 4.25-4.20 (m, 2H), 4.16-4.14 (m, 2H), 4.12-4.05 (m, 2H), 3.58 (s, 2H), 3.56 (s, 1H), 2.46 (d, J=1.6 Hz, 2H), 2.39 (d, J=1.2 Hz, 1H).

### Compound Ib-28: (S*)-3-(4-(6-(2-chloro-3,4-difluorophenyl)-5-(ethoxycarbonyl)-2-(thiazol-2-yl)-3,6-dihydropyrimidin-4-yl)cuban-1-yl)-2,2-dimethylpropanoic Acid

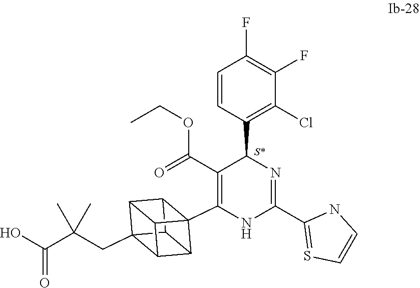

The synthesis of compound Ib-28 is similar with compound Ib-1.

LC-MS (ESI): mass calcd. for C₂₉H₂₆ClF₂N₃O₄S 585.1, m/z found 586.2 [M+H]⁺. ¹H NMR (400 MHz, CDCl₃) δ 8.03 (br s, 1H), 7.79 (d, J=2.8 Hz, 1H), 7.42 (d, J=3.2 Hz, 1H), 7.10-7.07 (m, 1H), 7.03-6.97 (m, 1H), 6.19 (s, 1H), 4.19-4.11 (m, 3H), 4.07-3.97 (m, 2H), 3.88-3.79 (m, 3H), 2.01 (s, 2H), 1.25 (s, 6H), 1.9 (t, J=7.2 Hz, 3H).

The following compounds were made according to the synthetic procedures described hereinabove:

TABLE 1

| | |
|---|---|
| 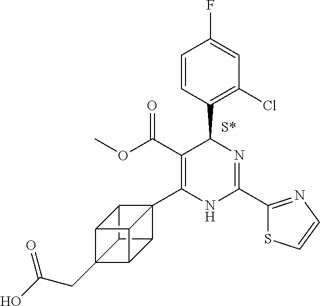 [SMILES: COC(=O)C1=C(C23C4C5C2C2C3C4C52CC(=O)O)NC(c2nccs2)=N[C@@H]1c1ccc(F)cc1Cl] | Ib-1 |
| 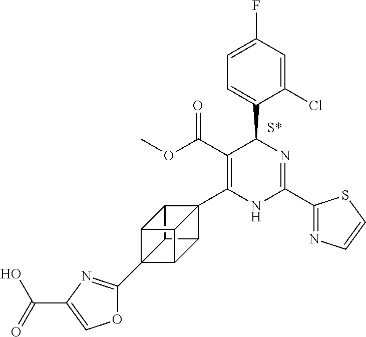 [SMILES: COC(=O)C1=C(C23C4C5C2C2C3C4C52c2nc(C(=O)O)co2)NC(c2nccs2)=N[C@@H]1c1ccc(F)cc1Cl] | Ib-2 |
| 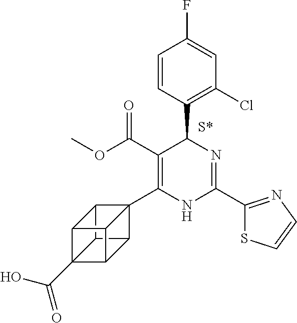 [SMILES: COC(=O)C1=C(C23C4C5C2C2C3C4C52C(=O)O)NC(c2nccs2)=N[C@@H]1c1ccc(F)cc1Cl] | Ib-3 |
| 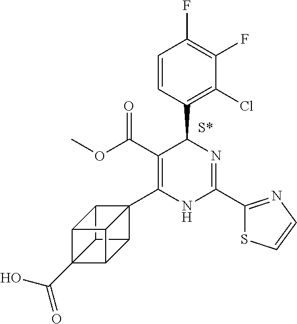 [SMILES: COC(=O)C1=C(C23C4C5C2C2C3C4C52C(=O)O)NC(c2nccs2)=N[C@@H]1c1ccc(F)c(F)c1Cl] | Ib-4 |
| 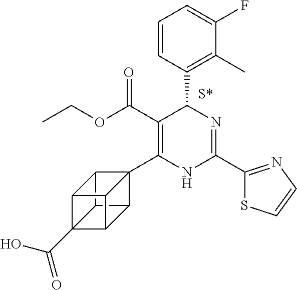 [SMILES: CCOC(=O)C1=C(C23C4C5C2C2C3C4C52C(=O)O)NC(c2nccs2)=N[C@H]1c1cccc(F)c1C] | Ib-5 |
| 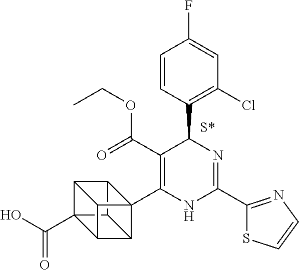 [SMILES: CCOC(=O)C1=C(C23C4C5C2C2C3C4C52C(=O)O)NC(c2nccs2)=N[C@@H]1c1ccc(F)cc1Cl] | Ib-6 |
| 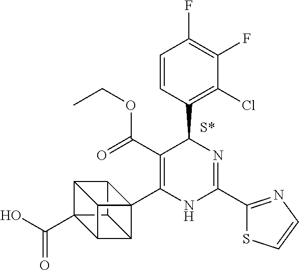 [SMILES: CCOC(=O)C1=C(C23C4C5C2C2C3C4C52C(=O)O)NC(c2nccs2)=N[C@@H]1c1ccc(F)c(F)c1Cl] | Ib-7 |
| 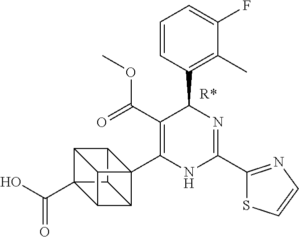 [SMILES: COC(=O)C1=C(C23C4C5C2C2C3C4C52C(=O)O)NC(c2nccs2)=N[C@@H]1c1cccc(F)c1C] | Ia-1 |
| 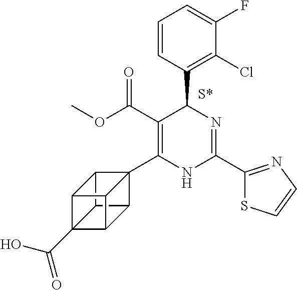 [SMILES: COC(=O)C1=C(C23C4C5C2C2C3C4C52C(=O)O)NC(c2nccs2)=N[C@@H]1c1cccc(F)c1Cl] | Ib-8 |
| 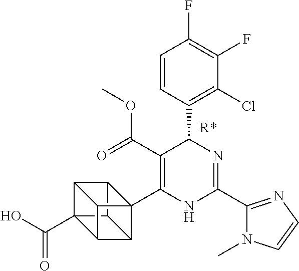 [SMILES: COC(=O)C1=C(C23C4C5C2C2C3C4C52C(=O)O)NC(c2nccn2C)=N[C@H]1c1ccc(F)c(F)c1Cl] | Ia-2 |
| 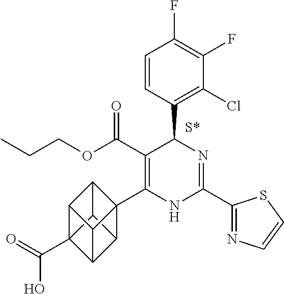 [SMILES: CCCOC(=O)C1=C(C23C4C5C2C2C3C4C52C(=O)O)NC(c2nccs2)=N[C@@H]1c1ccc(F)c(F)c1Cl] | Ib-11 |
| 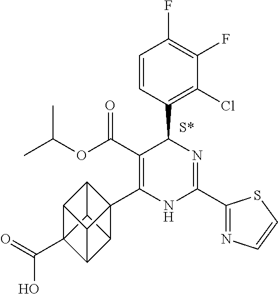 [SMILES: CC(C)OC(=O)C1=C(C23C4C5C2C2C3C4C52C(=O)O)NC(c2nccs2)=N[C@@H]1c1ccc(F)c(F)c1Cl] | Ib-12 |
| 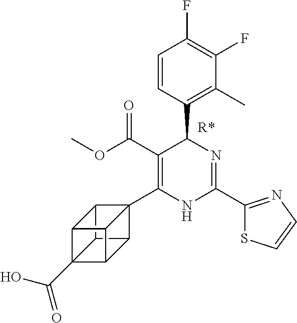 [SMILES: COC(=O)C1=C(C23C4C5C2C2C3C4C52C(=O)O)NC(c2nccs2)=N[C@@H]1c1ccc(F)c(F)c1C] | Ia-3 |
| 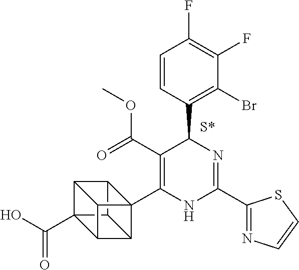 [SMILES: COC(=O)C1=C(C23C4C5C2C2C3C4C52C(=O)O)NC(c2nccs2)=N[C@@H]1c1ccc(F)c(F)c1Br] | Ib-13 |
| 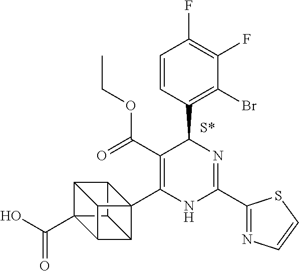 [SMILES: CCOC(=O)C1=C(C23C4C5C2C2C3C4C52C(=O)O)NC(c2nccs2)=N[C@@H]1c1ccc(F)c(F)c1Br] | Ib-14 |
| 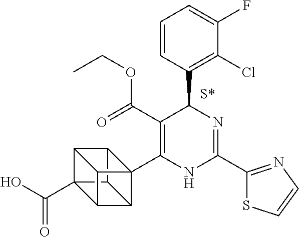 [SMILES: CCOC(=O)C1=C(C23C4C5C2C2C3C4C52C(=O)O)NC(c2nccs2)=N[C@@H]1c1cccc(F)c1Cl] | Ib-15 |
| 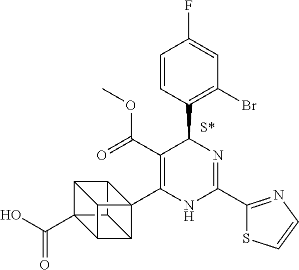 [SMILES: COC(=O)C1=C(C23C4C5C2C2C3C4C52C(=O)O)NC(c2nccs2)=N[C@@H]1c1ccc(F)cc1Br] | Ib-16 |
| 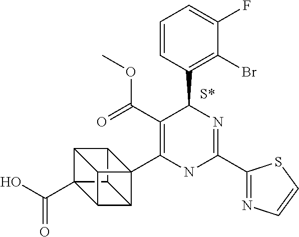 [SMILES: COC(=O)C1=C(C23C4C5C2C2C3C4C52C(=O)O)NC(c2nccs2)=N[C@@H]1c1cccc(F)c1Br] | Ib-17 |
| 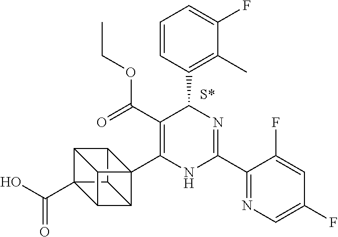 [SMILES: CCOC(=O)C1=C(C23C4C5C2C2C3C4C52C(=O)O)NC(c2ncc(F)cc2F)=N[C@H]1c1cccc(F)c1C] | Ib-18 |
| 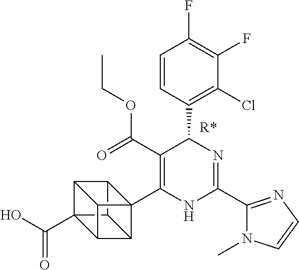 [SMILES: CCOC(=O)C1=C(C23C4C5C2C2C3C4C52C(=O)O)NC(c2nccn2C)=N[C@H]1c1ccc(F)c(F)c1Cl] | Ia-4 |
| 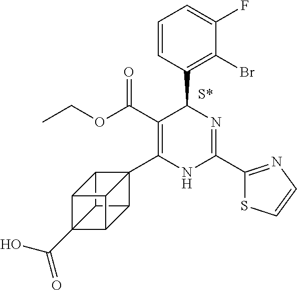 [SMILES: CCOC(=O)C1=C(C23C4C5C2C2C3C4C52C(=O)O)NC(c2nccs2)=N[C@@H]1c1cccc(F)c1Br] | Ib-19 |
| 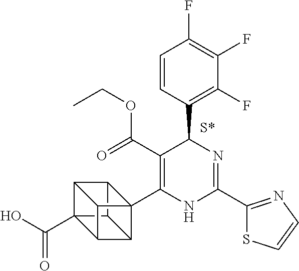 [SMILES: CCOC(=O)C1=C(C23C4C5C2C2C3C4C52C(=O)O)NC(c2nccs2)=N[C@@H]1c1ccc(F)c(F)c1F] | Ib-20 |
| 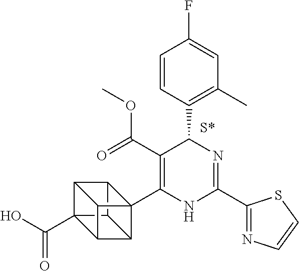 [SMILES: COC(=O)C1=C(C23C4C5C2C2C3C4C52C(=O)O)NC(c2nccs2)=N[C@H]1c1ccc(F)cc1C] | Ib-21 |
| 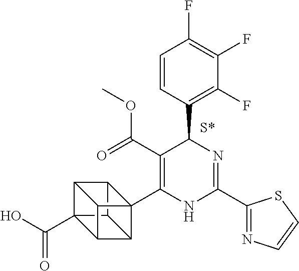 [SMILES: COC(=O)C1=C(C23C4C5C2C2C3C4C52C(=O)O)NC(c2nccs2)=N[C@@H]1c1ccc(F)c(F)c1F] | Ib-22 |
| 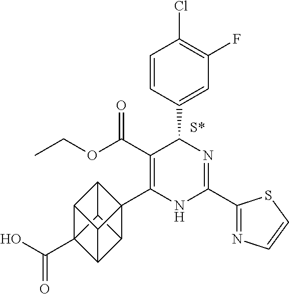 [SMILES: CCOC(=O)C1=C(C23C4C5C2C2C3C4C52C(=O)O)NC(c2nccs2)=N[C@H]1c1ccc(Cl)c(F)c1] | Ib-23 |
| 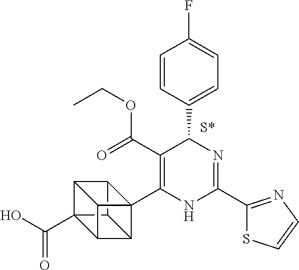 [SMILES: CCOC(=O)C1=C(C23C4C5C2C2C3C4C52C(=O)O)NC(c2nccs2)=N[C@H]1c1ccc(F)cc1] | Ib-24 |
| 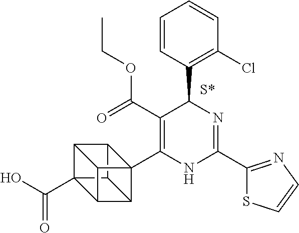 [SMILES: CCOC(=O)C1=C(C23C4C5C2C2C3C4C52C(=O)O)NC(c2nccs2)=N[C@@H]1c1ccccc1Cl] | Ib-25 |
| 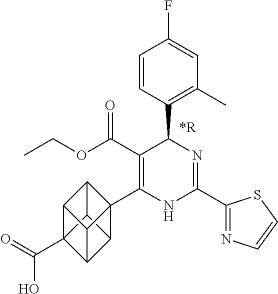 [SMILES: CCOC(=O)C1=C(C23C4C5C2C2C3C4C52C(=O)O)NC(c2nccs2)=N[C@@H]1c1ccc(F)cc1C] | Ia-5 |
| 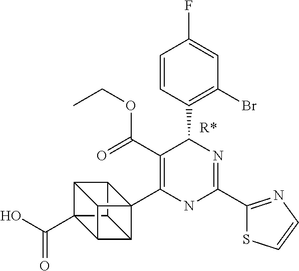 [SMILES: CCOC(=O)C1=C(C23C4C5C2C2C3C4C52C(=O)O)NC(c2nccs2)=N[C@H]1c1ccc(F)cc1Br] | Ia-6 |
| 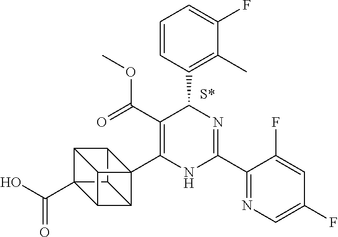 [SMILES: COC(=O)C1=C(C23C4C5C2C2C3C4C52C(=O)O)NC(c2ncc(F)cc2F)=N[C@H]1c1cccc(F)c1C] | Ib-26 |
| 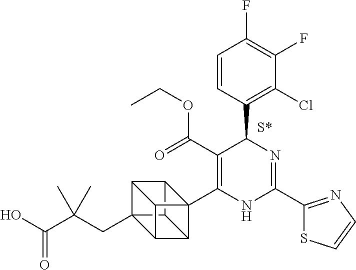 [SMILES: CCOC(=O)C1=C(C23C4C5C2C2C3C4C52CC(C)(C)C(=O)O)NC(c2nccs2)=N[C@@H]1c1ccc(F)c(F)c1Cl] | Ib-28 |

### Example 2: Anti-Viral Assay in HepG2.2.15 Cells

Materials and Equipments

1) Cell Line

HepG2.2.15 (the HepG2.2.15 cell line can be produced by transfection of the HepG2 cell line as described in Sells, Chen, and Acs 1987 (Proc. Natl. Acad. Sci. USA 84: 1005-1009), and the HepG2 cell line is available from ATCC® under number HB-8065™)

2) Reagents

DMEM/F12 (INVITROGEN-11330032)

FBS (GIBCO-10099-141)

Dimethyl sulfoxide (DMSO) (SIGMA-D2650)

Penicillin-streptomycin solution (HYCLONE-SV30010)

NEAA (INVITROGEN-1114050)

L-Glutamine (INVITROGEN-25030081)

Geneticin Selective Antibiotic (G418, 500 mg/ml) (INVITROGEN-10131027)

Trypsinase digestion solution (INVITROGEN-25300062)

CCK8 (BIOLOTE-35004)

QIAamp 96 DNA Blood Kit (12) (QIAGEN-51162)

FastStart Universal Probe Mast Mix (ROCHE-04914058001)

3) Consumables

96-well cell culture plate (COSTAR-3599)

Micro Amp Optical 96-well reaction plate (APPLIED BIOSYSTEMS-4306737)

Micro Amp Optical 384-well reaction plate (APPLIED BIOSYSTEMS)

4) Equipment

Plate reader (MOLECULAR DEVICES, SPECTRAMAX M2e)

Centrifuge (BECKMAN, ALLEGRA-X15R)

Real Time PCR system (APPLIED BIOSYSTEMS, QUANTSTUDIO 6)

Real Time PCR system (APPLIED BIOSYSTEMS, 7900HT)

Methods

1) Anti-HBV Activity and Cytotoxicity Determination

HepG2.2.15 cells were plated into 96-well plate in 2% FBS culture medium at the density of 40,000 cells/well and 5,000 cells/well for HBV inhibitory activity and cytotoxicity determination, respectively. After incubation at 37° C., 5% CO₂ overnight, cells were treated with medium containing compounds for 6 days with medium and compounds refreshed after 3 days of treatment. Each compound was tested in a 1:3 serial dilutions at 8 different concentrations in triplicate. The highest concentration of the compounds was 10 uM or 1 uM for anti-HBV activity assay and 100 uM for cytotoxicity determination.

Cell viability was determined by CCK-8 assay. After 6 days of compounds treatment, 20 μl CCK-8 reagents were added to each well of cytotoxicity assay plates. Cell plates were incubated at 37° C., 5% CO2 for 2.5 h. The absorbance at 450 nm wavelength and the absorbance at 630 nm wavelength as reference was measured.

The change of HBV DNA level induced by the compounds was assessed by quantitative real-time polymerase chain reaction (qPCR). Briefly, the HBV DNA in the culture medium was extracted using QIAamp 96 DNA Blood Kit according to the manual and then quantified by real-time PCR assay using the primers and probe in the table 1 below.

TABLE 2

| Primers | | SEQ |
|---|---|---|
| or | | ID |
| Probe | Sequence | NO: |
| HBV-Fw | GTGTCTGCGGCGTTTTATCA | 1 |
| HBV-Rev | GACAAACGGGCAACATACCTT | 2 |
| HBV-Probe | CCTCTKCATCCTGCTGCTATG | 3 |
| With FAM reporter | CCTCATC | |
| and TAMRA | | |
| quencher | | |

2) DATA Analysis

EC₅₀ and CC₅₀ values are calculated by the GRAPHPAD PRISM software. If the CV % of DMSO controls is below 15% and the reference compounds shows expected activity or cytotoxicity, the data of this batch of experiment is considered qualified.

RESULTS: See Table 3 below

TABLE 3

| Compound ID | CC₅₀ (μM) | EC₅₀ (μM) |
|---|---|---|
| Ib-1 | 0.36 | 42 |
| Ib-2 | 0.11 | 24 |
| Ib-3 | 0.084 | 70 |
| Ib-4 | 0.022 | 51 |
| Ib-5 | 0.017 | 49 |
| Ib-6 | 0.018 | >50 |
| Ib-7 | 0.005 | 25 |
| Ia-1 | 0.204 | >50 |
| Ib-8 | 0.132 | >50 |
| Ia-2 | 0.295 | >100 |
| Ib-11 | 0.052 | 22 |
| Ia-3 | 0.045 | >50 |
| Ib-12 | 0.037 | 28 |
| Ib-13 | 0.027 | >25 |
| Ib-14 | 0.007 | 41 |
| Ib-15 | 0.019 | >50 |
| Ib-16 | 0.071 | >50 |
| Ib-17 | 0.125 | 25 |
| Ib-18 | 0.061 | >12.5 |
| Ia-4 | 0.055 | >50 |
| Ib-19 | 0.0125 | >25 |
| Ib-20 | 0.028 | 46 |
| Ib-21 | 0.25 | >50 |
| Ib-22 | 0.282 | >25 |
| Ib-23 | 0.013 | >6.25 |
| Ib-24 | 0.223 | >25 |
| Ib-25 | 0.065 | >50 |
| Ia-5 | 0.043 | >50 |
| Ia-6 | 0.0115 | >12.5 |
| Ib-26 | 0.16 | >12.5 |
| Ib-28 | 0.0265 | 16 |

As the potency data shown in table 3, all these compounds demonstrated highly potent in vitro activities against HBV HepG2.2.15 cell.

1. A compound of formula (I) 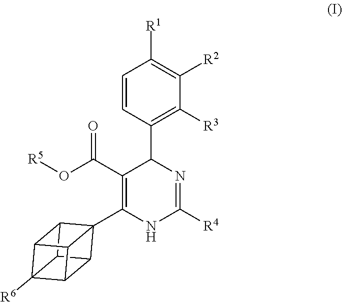
or a deuterated form, stereoisomer or tautomer thereof, wherein:
R¹, R² and R³ are each independently selected from the group consisting of hydrogen, C₁₋₃alkyl and halogen;
R⁴ is selected from the group consisting of thiazolyl, imidazolyl, oxazolyl and pyridyl, each of which is optionally substituted with one or more substituents each independently selected from methyl or halo;
R⁵ is C₁₋₄alkyl;
R⁶ is selected from the group consisting of —CO₂R^{x}, —C₁₋₉alkyl-CO₂R^{x}, and -Het-CO₂R^{x}; wherein
The C₁₋₉alkyl is optionally substituted with one or more substituents, each independently selected from halo and hydroxyl;
R^{x} is selected from H and —C₁₋₆alkyl; and
Het represents a 5- to 6-membered aromatic ring in which 1, 2, 3 or 4 of the ring members is a heteroatom each independently selected from the group consisting of N, O, and S, wherein said 5- to 6-membered aromatic ring is optionally substituted with one or more substituents, each independently selected from C₁₋₄alkyl and halo;
or a pharmaceutically acceptable salt or a solvate thereof.

2. The compound according to claim 1, wherein R¹, R² and R³ are each independently selected from the group consisting of H, halo, and methyl.

3. The compound according to claim 1, wherein R⁴ is selected from the group consisting of thiazolyl, oxazolyl, imidazolyl and pyridyl, each of which is optionally substituted with one methyl substituent.

4. The compound according to claim 1, wherein R⁵ is methyl or ethyl.

5. The compound according to claim 1, wherein R⁶ is selected from the group consisting of —CO₂H, —CH₂—CO₂H, and -Het-CO₂H.

6. The compound according to claim 1, wherein R⁶ is selected from the group consisting of —CO₂H, —CH₂—CO₂H, and -Het-CO₂H; wherein Het is a 5-membered aromatic ring selected from the group consisting of oxazolyl, thiazolyl, and imidazolyl, each of which is optionally substituted with one or more substituents, each independently selected from C₁₋₄alkyl and halo.

7. The compound according to claim 1, wherein
R¹, R² and R³ are each independently selected from the group consisting of hydrogen, methyl, fluoro, bromo, and chloro;
R⁴ is selected from the group consisting of thiazolyl, oxazolyl, imidazolyl and pyridyl, each of which is optionally substituted with one or more substituents each independently selected from methyl or halo;
R⁵ is methyl, ethyl, propyl, or iso-propyl; and
R⁶ is selected from the group consisting of —CO₂R^{x}, —C₁₋₆alkyl-CO₂R^{x}, and -Het-CO₂R^{x}; wherein
the C₁₋₆alkyl is optionally substituted with one or more substituents, each independently selected from halo and hydroxyl;
R^{x} is selected from H and —C₁₋₆alkyl; and
Het is a 5- to 6-membered aromatic ring selected from the group consisting of oxazolyl, thiazolyl, imidazolyl, pyridyl and pyrimidinyl, each of which is optionally substituted with one or more substituents, each independently selected from C₁₋₄alkyl and halo.

8. A compound selected from the group consisting of the following compounds: 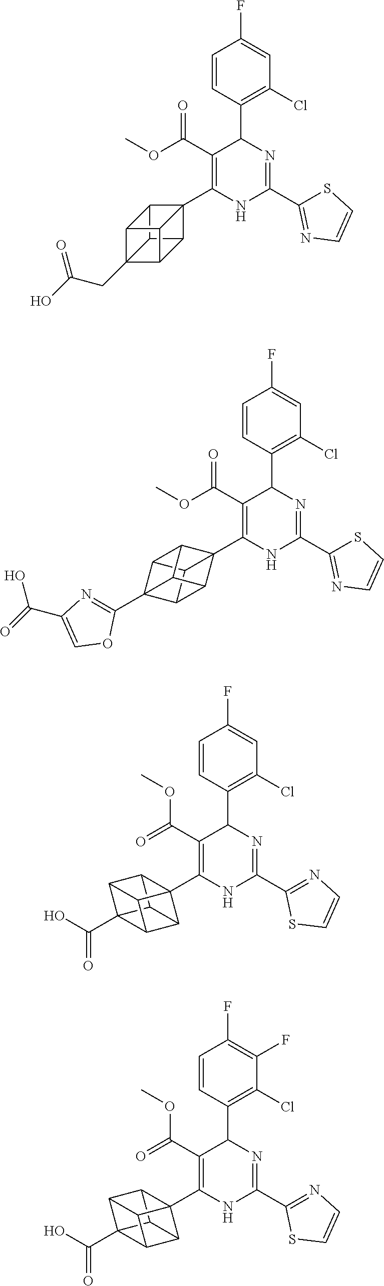 [SMILES: COC(=O)C1=C(C23C4C5C2C2C3C4C52CC(=O)O)NC(c2nccs2)=NC1c1ccc(F)cc1Cl ; COC(=O)C1=C(C23C4C5C2C2C3C4C52c2nc(C(=O)O)co2)NC(c2nccs2)=NC1c1ccc(F)cc1Cl ; COC(=O)C1=C(C23C4C5C2C2C3C4C52C(=O)O)NC(c2nccs2)=NC1c1ccc(F)cc1Cl ; COC(=O)C1=C(C23C4C5C2C2C3C4C52C(=O)O)NC(c2nccs2)=NC1c1ccc(F)c(F)c1Cl] 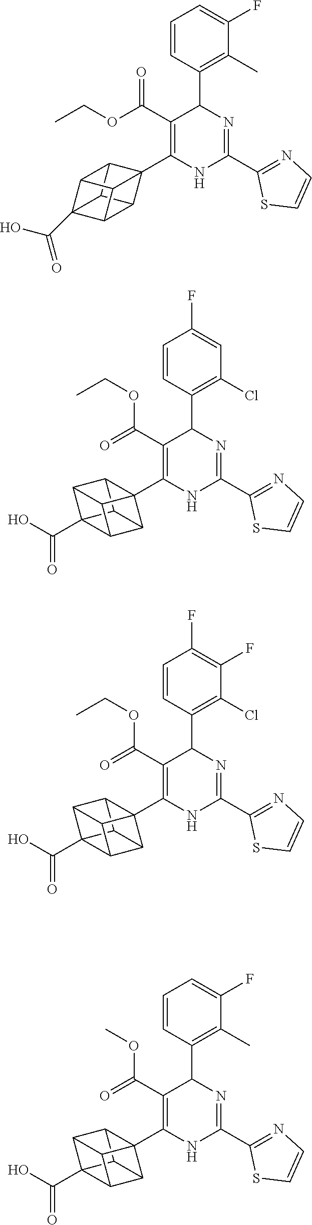 [SMILES: CCOC(=O)C1=C(C23C4C5C2C2C3C4C52C(=O)O)NC(c2nccs2)=NC1c1cccc(F)c1C ; CCOC(=O)C1=C(C23C4C5C2C2C3C4C52C(=O)O)NC(c2nccs2)=NC1c1ccc(F)cc1Cl ; CCOC(=O)C1=C(C23C4C5C2C2C3C4C52C(=O)O)NC(c2nccs2)=NC1c1ccc(F)c(F)c1Cl ; COC(=O)C1=C(C23C4C5C2C2C3C4C52C(=O)O)NC(c2nccs2)=NC1c1cccc(F)c1C] 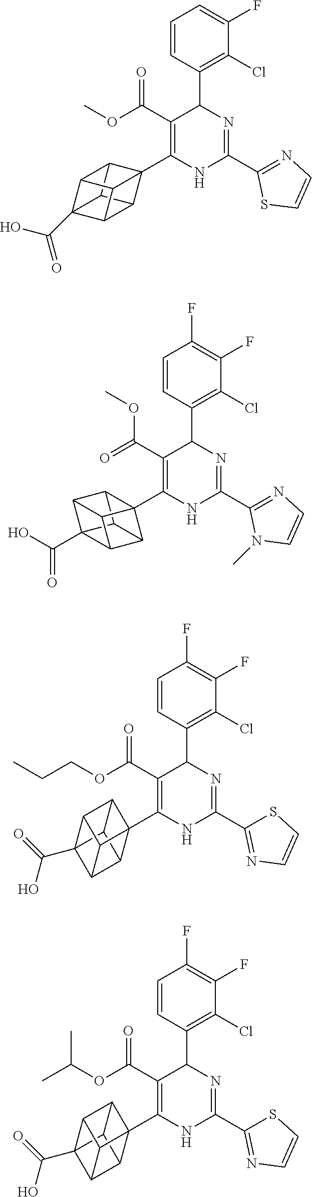 [SMILES: COC(=O)C1=C(C23C4C5C2C2C3C4C52C(=O)O)NC(c2nccs2)=NC1c1cccc(F)c1Cl ; COC(=O)C1=C(C23C4C5C2C2C3C4C52C(=O)O)NC(c2nccn2C)=NC1c1ccc(F)c(F)c1Cl ; CCCOC(=O)C1=C(C23C4C5C2C2C3C4C52C(=O)O)NC(c2nccs2)=NC1c1ccc(F)c(F)c1Cl ; CC(C)OC(=O)C1=C(C23C4C5C2C2C3C4C52C(=O)O)NC(c2nccs2)=NC1c1ccc(F)c(F)c1Cl] 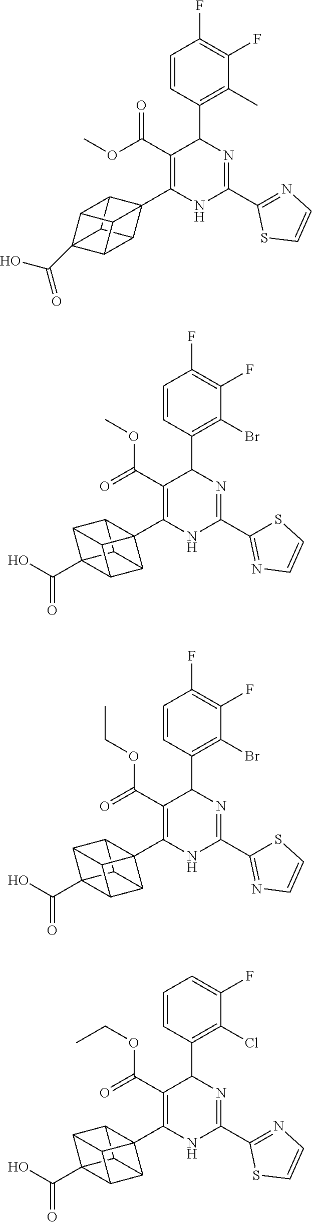 [SMILES: COC(=O)C1=C(C23C4C5C2C2C3C4C52C(=O)O)NC(c2nccs2)=NC1c1ccc(F)c(F)c1C ; COC(=O)C1=C(C23C4C5C2C2C3C4C52C(=O)O)NC(c2nccs2)=NC1c1ccc(F)c(F)c1Br ; CCOC(=O)C1=C(C23C4C5C2C2C3C4C52C(=O)O)NC(c2nccs2)=NC1c1ccc(F)c(F)c1Br ; CCOC(=O)C1=C(C23C4C5C2C2C3C4C52C(=O)O)NC(c2nccs2)=NC1c1cccc(F)c1Cl] 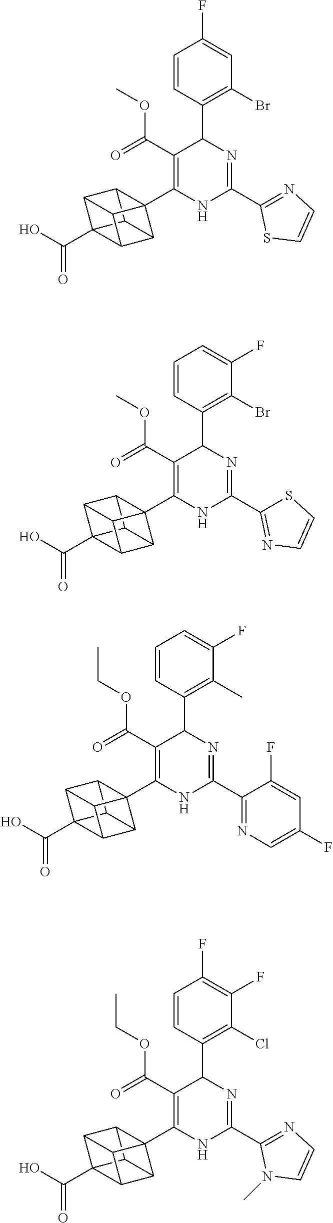 [SMILES: COC(=O)C1=C(C23C4C5C2C2C3C4C52C(=O)O)NC(c2nccs2)=NC1c1ccc(F)cc1Br ; COC(=O)C1=C(C23C4C5C2C2C3C4C52C(=O)O)NC(c2nccs2)=NC1c1cccc(F)c1Br ; CCOC(=O)C1=C(C23C4C5C2C2C3C4C52C(=O)O)NC(c2ncc(F)cc2F)=NC1c1cccc(F)c1C ; CCOC(=O)C1=C(C23C4C5C2C2C3C4C52C(=O)O)NC(c2nccn2C)=NC1c1ccc(F)c(F)c1Cl] 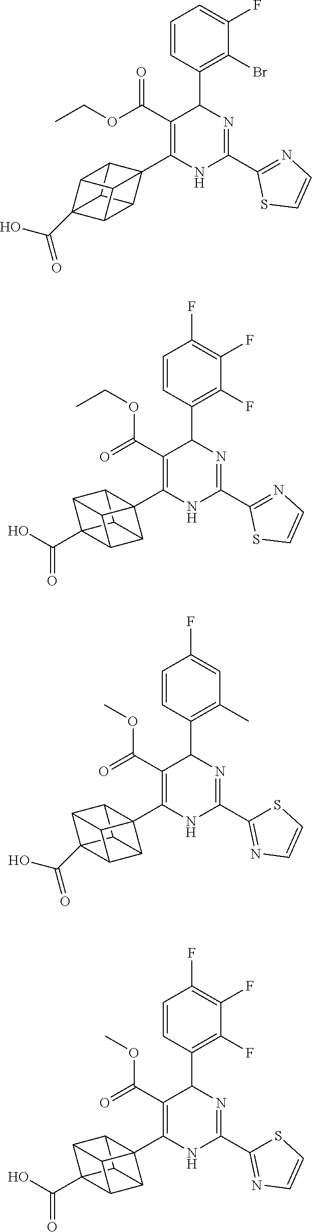 [SMILES: CCOC(=O)C1=C(C23C4C5C2C2C3C4C52C(=O)O)NC(c2nccs2)=NC1c1cccc(F)c1Br ; CCOC(=O)C1=C(C23C4C5C2C2C3C4C52C(=O)O)NC(c2nccs2)=NC1c1ccc(F)c(F)c1F ; COC(=O)C1=C(C23C4C5C2C2C3C4C52C(=O)O)NC(c2nccs2)=NC1c1ccc(F)cc1C ; COC(=O)C1=C(C23C4C5C2C2C3C4C52C(=O)O)NC(c2nccs2)=NC1c1ccc(F)c(F)c1F] 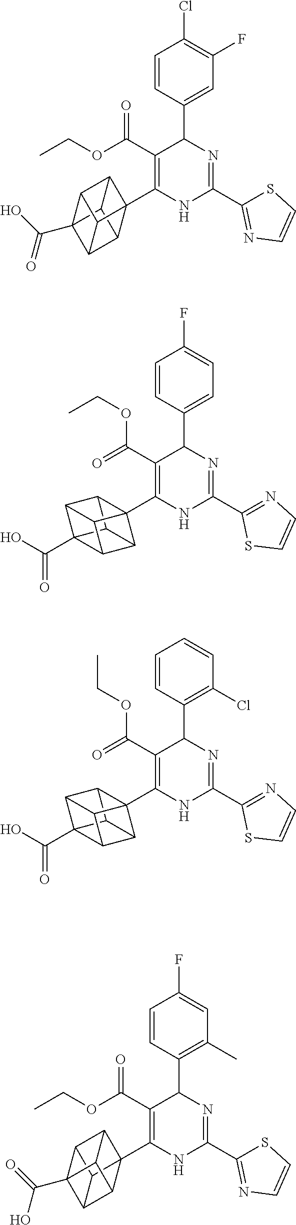 [SMILES: CCOC(=O)C1=C(C23C4C5C2C2C3C4C52C(=O)O)NC(c2nccs2)=NC1c1ccc(Cl)c(F)c1 ; CCOC(=O)C1=C(C23C4C5C2C2C3C4C52C(=O)O)NC(c2nccs2)=NC1c1ccc(F)cc1 ; CCOC(=O)C1=C(C23C4C5C2C2C3C4C52C(=O)O)NC(c2nccs2)=NC1c1ccccc1Cl ; CCOC(=O)C1=C(C23C4C5C2C2C3C4C52C(=O)O)NC(c2nccs2)=NC1c1ccc(F)cc1C] 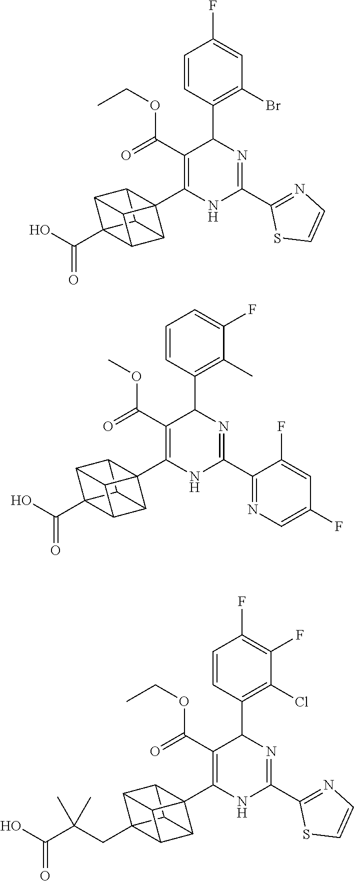 [SMILES: CCOC(=O)C1=C(C23C4C5C2C2C3C4C52C(=O)O)NC(c2nccs2)=NC1c1ccc(F)cc1Br ; COC(=O)C1=C(C23C4C5C2C2C3C4C52C(=O)O)NC(c2ncc(F)cc2F)=NC1c1cccc(F)c1C ; CCOC(=O)C1=C(C23C4C5C2C2C3C4C52CC(C)(C)C(=O)O)NC(c2nccs2)=NC1c1ccc(F)c(F)c1Cl]
or a deuterated form, stereoisomer or tautomer thereof, or a pharmaceutically acceptable salt or a solvate thereof.

9. A pharmaceutical composition comprising the compound of claim 1 and at least one pharmaceutically acceptable carrier.

10. (canceled)

11. A method of preventing ot treating an HBV infection or an HBV-induced disease in a mammal in need thereof, the method comprising administering to the mammal the pharmaceutical composition according to claim 9.

12. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the compound of claim 1.

13. A process for producing a compound of Formula (I) according to claim 1, the process comprising
(i) reacting a compound of Formula (IV) 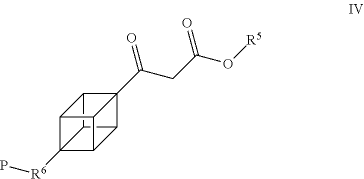
with a compound of formula (V) 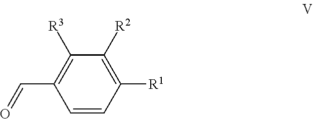
under suitable condensation conditions to yield a compound of Formula (X) 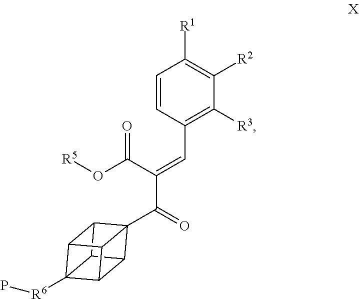
and
reacting the compound of Formula (X) with a compound of Formula (VI) 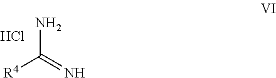
under basic conditions to generate a compound of Formula (VII) 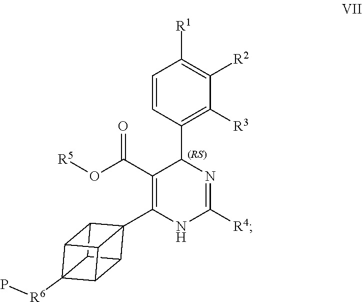
or alternatively
(ii) reacting the compounds of Formula (IV), (V) and (VI) in the presence of a suitable base in a suitable solvent to yield the compound of formula (VII); and optionally comprising the step of
(iii) resolving the compound of Formula (VII) into compounds of Formula (VIIa) and (VIIb) 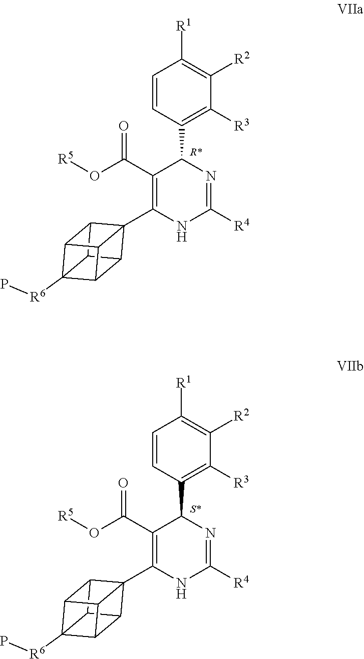
by chiral separation; and the step of
(iv) cleaving the protecting group in compound (VII) to yield compound of Formula (I); or
cleaving the protecting group in compound (VIIa) or compound (VIIb) to yield a compound of Formula (Ia) or (Ib) 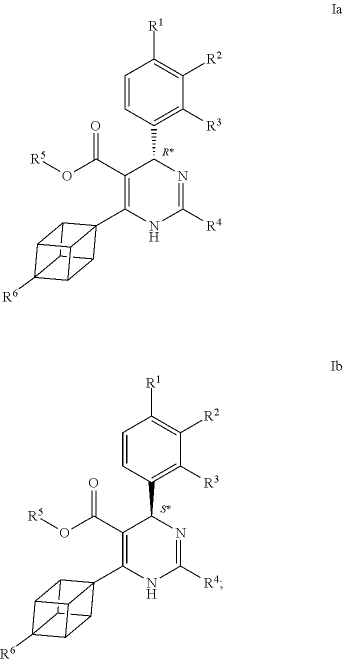
wherein R¹, R², R³, R⁴, R⁵, and R⁶ are as defined in claim 1, and P is a suitable protecting group.

14. A method of treating an HBV infection in an individual in need thereof, comprising administering to the individual a therapeutically effective amount of the pharmaceutical composition of claim 9.

15. A process for preparing the pharmaceutical composition of claim 9, comprising mixing at least one pharmaceutically acceptable carrier with a therapeutically effective amount of a compound of formula (I).

16. A method of preventing or treating an HBV infection or of an HBV-induced disease in mammal in need thereof, the method comprising administering to the mammal the compound according to claim 1.

17. A product comprising a first compound and a second compound as a combined preparation for simultaneous, separate or sequential use in the prevention or treatment of an HBV infection or of an HBV-induced disease in mammal in need thereof, wherein said first compound is different from said second compound, wherein said first compound is the pharmaceutical composition of claim 9.

18. A method of treating an HBV infection in an individual in need thereof, comprising administering to the individual a therapeutically effective amount of the compound according to claim 1.
